(19) United States
(12) Patent Application Publication
(10) Pub. No.: US 2019/0077797 A1
(43) Pub. Date: Mar. 14, 2019

(54) PROCESSES FOR PREPARING 2-DIHALO RIBOLACTONES

(71) Applicants: Ozkan SARI, Decatur, GA (US); Coralie DE SCHUTTER, Atlanta, GA (US); Jong-Hyun CHO, Snellville, GA (US); Longhu ZHOU, Atlanta, GA (US); Hongwang ZANG, Atlanta, GA (US); Franck AMBLARD, Atlanta, GA (US); Steven J. COATS, Tucker, GA (US); Raymond F. SCHINAZI, Miami, FL (US)

(72) Inventors: Ozkan SARI, Decatur, GA (US); Coralie DE SCHUTTER, Atlanta, GA (US); Jong-Hyun CHO, Snellville, GA (US); Longhu ZHOU, Atlanta, GA (US); Hongwang ZANG, Atlanta, GA (US); Franck AMBLARD, Atlanta, GA (US); Steven J. COATS, Tucker, GA (US); Raymond F. SCHINAZI, Miami, FL (US)

(21) Appl. No.: 15/772,294

(22) PCT Filed: Oct. 31, 2016
(86) PCT No.: PCT/US2016/059721

§ 371 Date: Apr. 30, 2018

Related U.S. Application Data
(60) Provisional application No. 62/248,652, filed on Oct. 30, 2015.

(51) Int. Cl. C07D 413/06; C07D 317/30; C07D 307/33

(52) U.S. Cl. CPC C07D 413/06; A61K 31/708; C07D 307/33; C07D 317/30

(57) ABSTRACT

Methods for forming 2-bromo, 2-fluoro ribofuranose intermediates and 2-chloro, 2-fluoro ribofuranose intermediates for use in preparing antiviral nucleosides are disclosed. Methods for forming nucleosides, and nucleoside prodrugs, using the intermediates, are also disclosed. The methods all produce intermediates, and the resulting nucleosides and prodrugs thereof, wherein the chirality of the carbon at the 2-position is controlled. In some embodiments, the chemistry involves using chiral auxiliaries, such as (R)-2,2-dimethyl-1,3-dioxolane-4-carbaldehyde, and in other embodiments, the chemistry involves using chiral starting materials, such as D-xylose.

### FIELD OF THE INVENTION

This application relates to novel methods for preparing 2-dihalo ribolactones, that are useful as intermediates for preparing various 2′-dihalo nucleoside analogs, which nucleoside analogs are useful for treating a variety of diseases, including HCV infection.

### BACKGROUND OF THE INVENTION

Hepatitis C virus (HCV) has infected more than 170 million people worldwide. It is estimated that three to four million persons are newly infected each year, 70% of whom will develop chronic hepatitis. HCV is responsible for 50-76% of all liver cancer cases, and two thirds of all liver transplants in the developed world. There is an urgent need for new HCV drugs that are potent and safe.

Hepatitis C virus genome comprises a positive-strand RNA enclosed in a nucleocapsid and lipid envelope and consists of 9.6 kb ribonucleotides and has a single open reading frame (ORP) encoding which encodes a large polypeptide of about 3,000 amino acids (Dymock et al. Antiviral Chemistry & Chemotherapy 2000, 11, 79). NSSA is a nonstructural 56-58 kDa protein which modulates HCV replication as a component of replication complex. NSSA is highly phosphorylated by cellular protein kinases and the phosphorylation sites are conserved among HCV genotypes (Katze et al., 2001; Kim et al., 1999).

Despite the availability of a vaccine (Crit. Rev. Clin. Lab. Sci. 2004, 41, 391-427). Yellow fever virus (YFV) continues to be a serious human health concern, causing approximately 30,000 deaths each year. YFV is one of the most lethal viral infections of humans (Expert Rev. Vaccines 2005, 4, 553-574.). Of infected individuals approximately 15% will develop severe disease, with a fatality rate of 20 to 50% among those individuals. No approved therapies specific for treatment of YFV are available. Treatment is symptomatic-rest, fluids, and ibuprofen, naproxen, acetaminophen, or paracetamol may relieve symptoms of fever and aching. Aspirin should be avoided. Although the virus is endemic to Africa and South America, there is potential for outbreaks of YFV outside these areas and such imported cases have been reported (J. Travel Med. 2005, 12(Suppl. 1), S3-S11).

West Nile Virus (WNV) is from the family Flaviviridae and predominantly a mosquito-borne disease. It was first discovered in the West Nile District of Uganda in 1937. According to the reports from the Centers for Disease Control and Prevention, WNV has been found in Africa, the Middle East, Europe, Oceania, west and central Asia, and North America. Its first emergence in North America began in the New York City metropolitan area in 1999. It is a seasonal epidemic in North America that normally erupts in the summer and continues into the fall, presenting a threat to environmental health. Its natural cycle is bird-mosquito-bird and mammal. Mosquitoes, in particular the species Culex pipiens, become infected when they feed on infected birds. Infected mosquitoes then spread WNV to other birds and mammals including humans when they bite. In humans and horses, fatal Encephalitis is the most serious manifestation of WNV infection. WNV can also cause mortality in some infected birds. There is no specific treatment for WNV infection.

Dengue infection is also from the family Flaviviridae and is the most important arthropod-borne infection in Singapore (Epidemiol News Bull 2006, 32, 62-6). Globally, there are an estimated 50 to 100 million cases of dengue fever (DF) and several hundred thousand cases of dengue hemorrhagic fever (DHF) per year with and average fatality fate of 5%. Many patients recover from dengue infection with minimal or no residual illness. Dengue infections are usually asymptomatic, but can present with classic dengue fever, dengue hemorrhagic fever or dengue shock syndrome. Even for outpatients, the need for maintaining adequate hydration is highly important. Dengue infections can be effectively managed by intravenous fluid replacement therapy, and if diagnosed early, fatality rates can be kept below 1%. To manage the pain and fever, patients suspected of having a dengue infection should be given acetaminophen preparations. Aspirin and non-steroidal anti-inflammatory medications may aggravate the bleeding tendency associated with some dengue infection. However, some manifestations of dengue infection previously described include liver failure (Dig. Dis. Sci. 2005, 50, 1146-7), encephalopathy (J. Trop. Med. Public Health 1987, 18, 398-406), and Guillain-Barré syndrome (Intern Med. 2006, 45, 563-4).

Proliferative disorders are one of the major life-threatening diseases and have been intensively investigated for decades. Cancer now is the second leading cause of death in the United States, and over 500,000 people die annually from this proliferative disorder. A tumor is an unregulated, disorganized proliferation of cell growth. A tumor is malignant, or cancerous, if it has the properties of invasiveness and metastasis. Invasiveness refers to the tendency of a tumor to enter surrounding tissue, breaking through the basal laminas that define the boundaries of the tissues, thereby often entering the body's circulatory system. Metastasis refers to the tendency of a tumor to migrate to other areas of the body and establish areas of proliferation away from the site of initial appearance.

Cancer is not fully understood on the molecular level. It is known that exposure of a cell to a carcinogen such as certain viruses, certain chemicals, or radiation, leads to DNA alteration that inactivates a “suppressive” gene or activates an “oncogene.” Suppressive genes are growth regulatory genes, which upon mutation, can no longer control cell growth. Oncogenes are initially normal genes (called prooncogenes) that by mutation or altered context of expression become transforming genes. The products of transforming genes cause inappropriate cell growth. More than twenty different normal cellular genes can become oncogenes by genetic alteration. Transformed cells differ from normal cells in many ways, including cell morphology, cell-to-cell interactions, membrane content, cytoskeletal structure, protein secretion, gene expression and mortality (transformed cells can grow indefinitely).

All of the various cell types of the body can be transformed into benign or malignant tumor cells. The most frequent tumor site is lung, followed by colorectal, breast, prostate, bladder, pancreas and then ovary. Other prevalent types of cancer include leukemia, central nervous system cancers, including brain cancer, melanoma, lymphoma, erythroleukemia, uterine cancer, and head and neck cancer.

Chemotherapy involves the disruption of cell replication or cell metabolism. It is used most often in the treatment of leukemia, as well as breast, lung, and testicular cancer. There are five major classes of chemotherapeutic agents currently in use for the treatment of cancer: natural products and their derivatives; anthracyclines; alkylating agents; antiproliferatives (also called antimetabolites); and hormonal agents. Chemotherapeutic agents are often referred to as antineoplastic agents.

Several synthetic nucleosides that exhibit anticancer activity, such as 5-fluorouracil, have been identified. 5-Fluorouracil has been used clinically in the treatment of malignant tumors, including, for example, carcinomas, sarcomas, skin cancer, cancer of the digestive organs, and breast cancer. 5-Fluorouracil, however, causes serious adverse reactions such as nausea, alopecia, diarrhea, stomatitis, leukocytic thrombocytopenia, anorexia, pigmentation and edema.

U.S. provisional applications 61/984,036, 62/073,937, and 62/155,939 disclose 2′-dihalo nucleoside analogs and prodrugs with improved properties over known therapeutics. Additional 2′-dihalo nucleoside analogs and prodrugs are disclosed in international patent applications WO 2015/034420, WO 2015/056213 and WO 2015/081297, along with U.S. Publication No. 2015175648. All of these disclosures share problems controlling the stereochemistry at the 2-dihalo ribolactone center. This results in lower chemical yields and difficult separations.

There is a need in the art for improved methods for making 2-dihalo ribo- and deoxy-ribo sugars as they are useful as intermediates for preparing nucleoside analogs and nucleoside prodrugs. The present invention provides such methods.

### SUMMARY OF THE INVENTION

In one embodiment, the present invention is directed to a process for making compounds of Formula I:

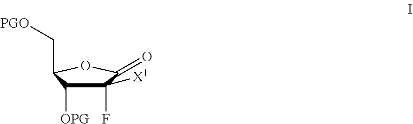

X¹ is selected from the group consisting of Cl, Br, and I, and PG is a hydroxyl protecting group.

These compounds are useful intermediates for preparing antiviral nucleosides with F and X¹ substitution at the 2-position. In one aspect of this embodiment, the chemistry described herein allows for efficient control of the stereochemistry at the 2-position.

In each of the embodiments described herein, those of skill in the art will readily understand, from the reactants, the types of reactions, the intermediates, and the final products, that a variety of acids, bases, protecting groups, leaving groups, and solvents can be used to carry out the conversions. While explanations of the chemistry are provided, and exemplary acids, bases, protecting groups, leaving groups, and solvents are disclosed, this is in no way intended to limit the chemistry to these examples.

In certain embodiments, where a drawing shows a particular carbon atom on a reactant as not having chirality (i.e., the bond is shown as a wavy line), and the product is indicated as having chirality, it is to be understood that some form of purification occurs after the chemical reaction has taken place. In most cases, a diastereomeric mixture (R,R; R,S; S,R; and S,S) is formed, and there are several methods for separating the diastereomers. Those of skill in the art can readily determine an appropriate method for separating the diastereomers, such as crystallization, chromatography, enzymatic resolution, and the like.

In the embodiments described herein, stereochemical control at the 2-carbon is provided by employing reactants and/or starting materials that have a chiral center, which acts as a chiral director for the stereochemistry at the 2-carbon.

In one aspect of this embodiment, the method initially involves reacting a 4-(S)-oxazolidinone such as Formula II with an X¹-substituted acetyl group to form an imide of Formula III.

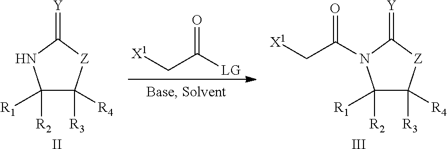

wherein:

Y is O or S,

Z is O, S, N-alkyl, N-aryl, N-alkylaryl, or N-arylalkyl;

R¹, R², R³ and R⁴ are, independently, selected from the group consisting of H, C₁₋₁₀ alkyl, C₁₋₁₀ branched alkyl, C₃₋₆ cycloalkyl, C₁₋₄(alkyl)aryl, benzyl, C₁₋₃(alkyl)C—O-aryl, CH₂OBz (where Bz=benzyl), aryl, heteroaryl and C₃₋₆ heterocycloalkyl;

R¹ and R² or R³ and R⁴ can optionally be linked to form a C₃₋₈ cycloalkyl, alkyl substituted C₃₋₈ cycloalkyl or C₃₋₈ cycloalkyl fused to aryl; and

LG is a leaving group.

The base has to be sufficiently basic to deprotonate the NH moiety in the compound of Formula II, so it can displace the leaving group on the compound of Formula III, and, ideally, too hindered to displace the X¹ substituent. Grignard and organolithium bases are sufficiently basic.

The solvent is an organic solvent.

Compounds of Formula III are allowed to react under basic conditions with (R)-2,2-dimethyl-1,3-dioxolane-4-carbaldehyde to provide a compound of Formula IV.

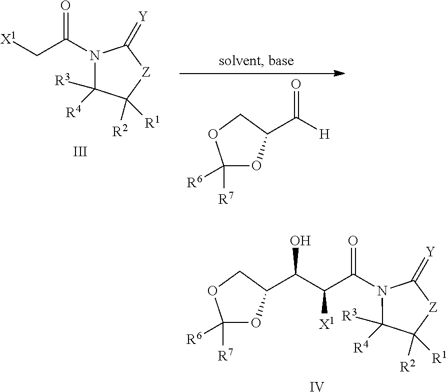

R⁶ and R⁷ are, independently, selected from the group consisting of C₁₋₁₀ alkyl, aryl, C₁₋₄ (alkyl)aryl, such as benzyl, heteroaryl and —(C₁₋₄ alkyl)-heteroaryl;

R⁶ and R⁷ optionally can be linked to form C₃₋₈ cycloalkyl, C₅₋₈ cycloheteroalkyl, alkyl substitute C₃₋₈ cycloalkyl and C₃₋₈ cycloalkyl fused to aryl.

The chemistry involves deprotonating the —CHX¹— moiety alpha to the carbonyl, and the resulting enolate ion reacts with the aldehyde on the dioxolane-4-carboxaldehyde. It is important to select a sterically-hindered base, to deprotanate without performing a nucleophilic displacement of the X1 moiety. Lithium amides such as LDA are preferred bases.

The reaction is preferably carried out in a polar, aprotic solvent.

The chirality of the dioxolane-4-carboxaldehyde and oxazolidinone drives the stereochemical preference in this reaction, which allows for the stereoselective fluorination in a later step.

In the event that the chemistry does not provide a single diasteromer, the compound can be purified at this stage if desired, using known techniques, to provide Compound IV as a relatively pure diasteromer.

Alternatively, a compound of Formula III can be reacted under basic conditions, in an aprotic solvent, and in the presence of a Lewis acid, with (R)-2,2-dimethyl-1,3-dioxolane-4-carbaldehyde to provide a compound of Formula IV. Examples of such reagent combinations include (c-hexyl)₂BOTf, Et₃N, Bu₂BOTf, Et₃N (or lutidine) and 9-BBN-OTf, DIPEA, TiCl₄, TMEDA (or DIPEA).

The OH group in Compound IV is then suitably protected with a protecting group (PG) which is largely stable to both basic conditions and acidic conditions, such as a silyl protecting group, which can be deprotected using a fluoride anion, or a group like benzyl, which can be deprotected using hydrogen, to form a compound of Formula V. A representative reaction scheme is shown below:

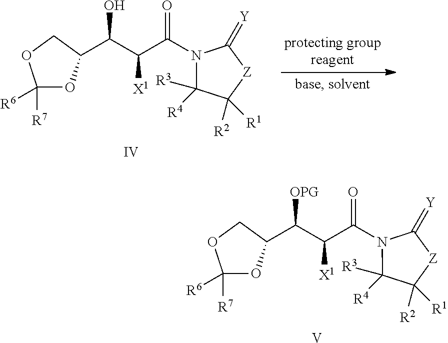

The formation of a protective group typically forms an acid by-product, so a base is present to neutralize the acid. The base is one which does not displace X¹, and is typically a secondary or tertiary amine.

The oxazolidinone chiral auxiliary in Formula V is then removed by reacting the compounds with an alkoxide of the alcohol (R⁵OM), where M is a metal, such as an alkali metal or alkaline earth metal, or a quaternary ammonium salt, and R⁵ is selected from the group consisting of C₁₋₁₀ alkyl, C₁₋₁₀ branched alkyl C₃₋₆ cycloalkyl and alkyl substituted C₃₋₈ cycloalkyl, to provide an ester of Formula VI. The solvent can be, but need not be, the alcohol R⁵OH, as is shown below:

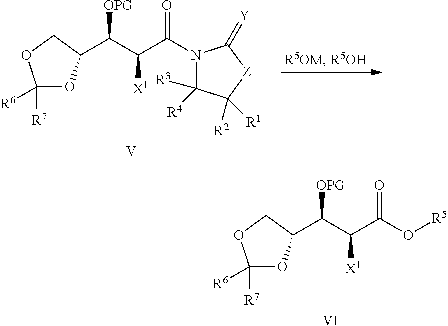

Selective introduction of fluorine represents a key step in the methods described herein, as the desired stereochemistry is obtained in high yield at what will be the 2 position of the desired ribonolactone. The —CHX¹— moiety alpha to the ester is deprotonated, in an aprotic solvent, using a hindered base, then reacted with an electrophilic fluorine reagent, to provide dihalo intermediates of Formula VII, as shown below:

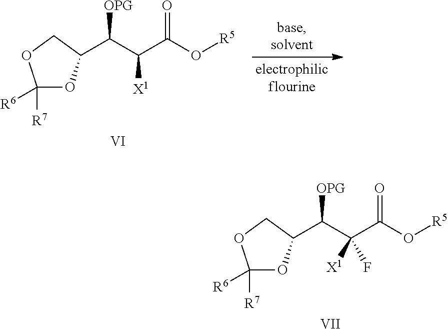

Intermediates of Formula VII can be cyclized to ribolactones of Formula VIII by reaction with an acid catalyst, as shown below:

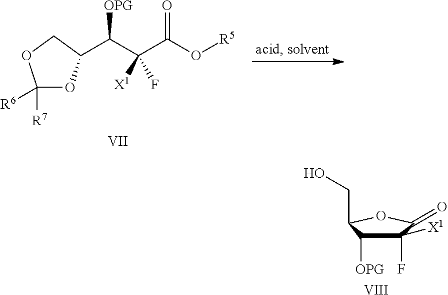

The free 5-alcohol group of VIII is then protected to give a ribonolactone of Formula I.

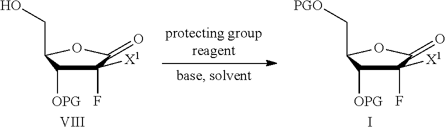

Alternatively, the workup of the reaction for forming compounds of Formula IV formation is modified to instead involve quenching the reaction with an alcohol (R⁵OH as defined above) to form an intermediate ester of Formula IXa as shown below. Some inconsequential scrambling of the carbon center to which X¹ is attached occurs during this step. The secondary alcohol of this intermediate can be protected to provide an intermediate of Formula VI. This approach reduces one step in the overall process, while still providing desired compounds of Formula I.

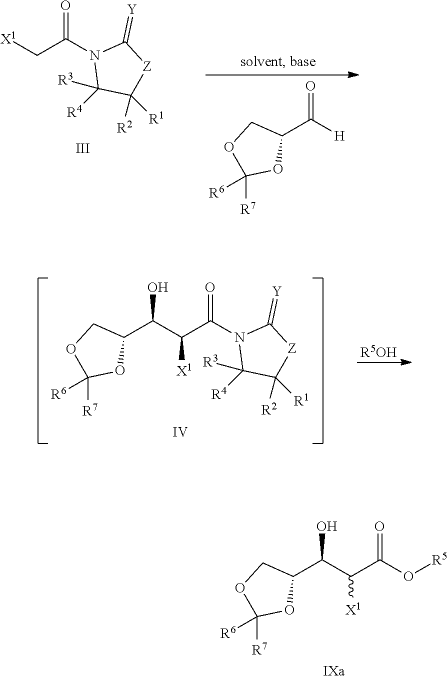

In a second embodiment, the method involves reacting an alpha halo acetate ester of Formula X with (R)-2,2-dimethyl-1,3-dioxolane-4-carbaldehyde in the presence of a base to provide compounds of Formula XI, in which the 3-OH is predominately in the R stereochemical configuration.

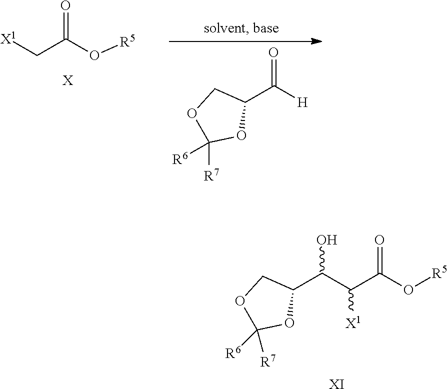

The base is strong enough to deprotonate alpha to the ester group, but hindered enough to not displace X¹, and lithium diisopropylamide (LDA) is preferred. The solvents are typically polar aprotic solvents, such as THF.

An alternate procedure which also provides the 3-OH predominately in the R stereochemical configuration involves reacting compounds of Formula XII with (R)-2,2-dimethyl-1,3-dioxolane-4-carbaldehyde in the presence of metals such as zinc or magnesium, or boranes, including trialkylboranes such as triethylborane, in a polar aprotic solvents, such as tetrahydrofuran (THF).

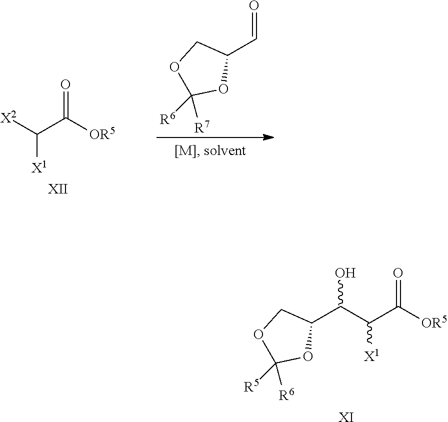

X² is a halogen selected from the group consisting of Cl, Br, and I, and X¹, R⁵, R⁶, and R⁷ are as described above.

The free hydroxyl group on the compounds of Formula XI is then protected to form diastereomeric compounds of Formula XIII The chirality introduced by the (R)-2,2-dimethyl-1,3-dioxolane-4-carbaldehyde reactant allows for a relatively higher amount of the desired diasteromer than the three other diasteromers, and once the hydroxyl group is protected, the desired diastereomer can be obtained using any of a variety of purification methods (not shown in the reaction below).

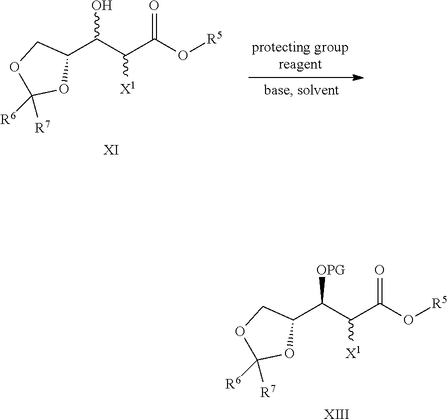

In this Scheme, R⁵, R⁶, R⁷, X¹ and PG are as defined in the earlier schemes.

The selective introduction of flourine represents a key step in this process, as the desired stereochemistry is obtained in high yield at what will be the 2 position of the desired ribolactone. The —CHX¹— moiety alpha to the ester is deprotonated using a hindered base, typically in a polar, aprotic solvent, and the resulting enolate is then reacted with an electrophilic fluorine reagent to provide dihalo intermediates of Formula XIV.

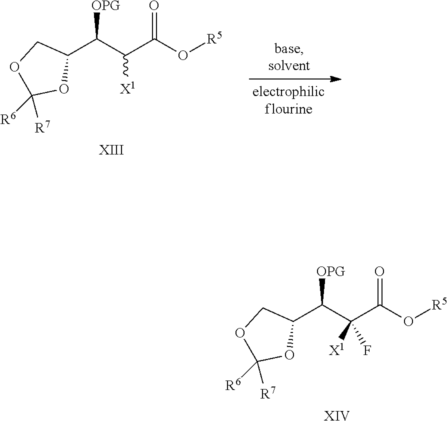

As with the earlier embodiment, intermediates of Formula XIV can be cyclized by reaction with an acid catalyst to form ribolactones of Formula I.

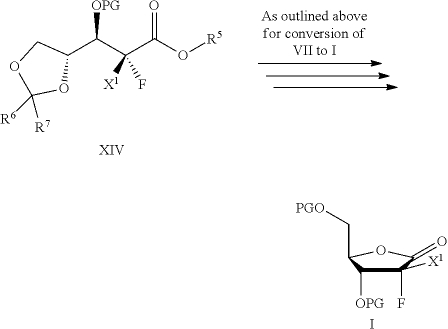

In a third embodiment, the method includes the following steps:

Compounds XV are oxidized with an oxidizing reagent such as sodium periodate (NaIO₄), followed by condensation with an α,β-unsaturated ester formation reagent, to form compounds XVI. Representative α, β-unsaturated ester formation reagents include alkyl trialkylphosphonoacetate and (carbalkoxymethylene)triarylphosphorane.

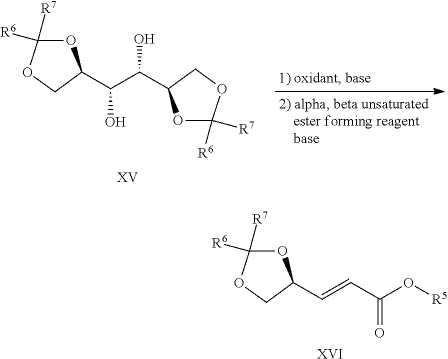

The carbon-carbon double bond is then converted to vicinal dihydroxyl groups by reacting with a dihydroxylation reagent, which is an inorganic or organic oxidizing reagent, such as AD-mix-β, OsO₄, Pb(OAc)₄, NaMnO₄, KMnO₄, with AD-mix-β being preferred.

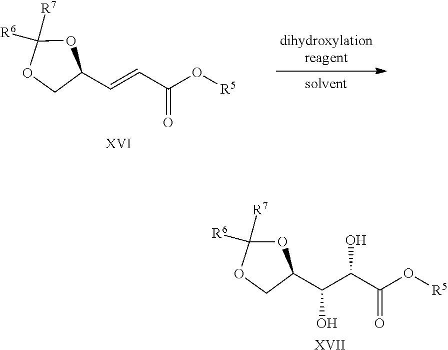

The α-hydroxyl group can be selectively converted into a leaving group, such as a sulfonyl ester. The formation of a leaving group forms an acid by-product, which is neutralized with a base suitable for neutralizing the acid, while not displacing X¹.

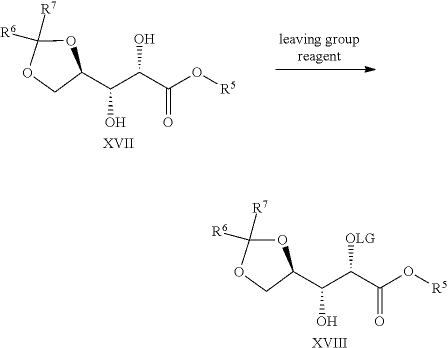

The leaving group of compound XVIII is displaced with a halogen, using halogenation reagents RX¹, to form a compound of Formula XIX. Halogenation reagents include inorganic and organic salts, in which X¹ is Cl, Br or I, and R is Li, Na, K, or NBu₄, with LiCl and LiBr being preferred. In one aspect of this embodiment, a crown ether, such as 18-crown-6, is added to accelerate the rate of reaction.

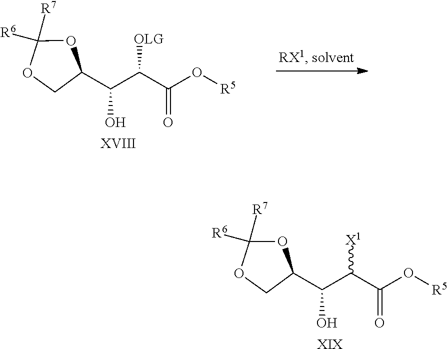

Compounds of Formula XIX can be converted to ribofuranoses of Formula I by the same methods as outlined above for the conversion of XI to I.

In a fourth embodiment, the method involves an initial step of forming a furanose intermediate (Formula XXI) in which the 1-hydroxy and 2-hydroxy groups are protected as a ketal or acetal, and the 3- and 5-hydroxy groups are protected as a different type of protecting group.

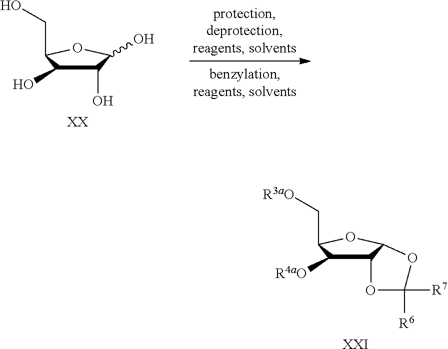

In this formula, R⁶ and R⁷ are as defined above. The R^{3a} and R^{4a} groups are preferably benzyl, but other suitable protecting groups, including, but not limited to, benzyl derivatives such as 4-halobenzyl, 4-methylbenzyl, 4-methoxybenzyl, 4-nitrobenzyl, 2,4-dimethylbenzyl groups, can also be used.

Alternatively, since compounds of the formula XXI are known, instead of performing process steps to form an intermediate of this type, one can use a similarly protected compound as the starting material for subsequent process steps. The latter may be preferred if such compounds are commercially available. For example, 1,2-O-Isopropylidene-D-ribofuranose is commercially available, and the 3- and 5-hydroxyl groups on this compound can be protected.

As will be appreciated in subsequent process steps, the ketal or acetal can be hydrolyzed in the presence of the protecting groups on the 3- and 5-hydroxy groups. There are several pathways to providing a suitably protected D-xylose of formula XXI. A few of these routes are discussed below.

In one aspect of this embodiment, D-xylose is reacted with a ketone or aldehyde to form a ketal or acetal. The typical conditions for forming a ketal or acetal with the 1- and 2-hydroxy groups on D-xylose also tend to form a ketal or acetal with the 3- and 5-hydroxy groups. However, it is possible to hydrolyze the ketal or acetal in a regioselective manner using a catalytic amount of an acid, and retain the ketal at the 1- and 2-positions, thus freeing up the hydroxyl groups at the 3- and 5-positions. These 3- and 5-hydroxyl groups are then protected.

As an example, the selective protection of the 1- and 2-hydroxyl groups on furanoses such as D-xylose (Formula XX) with an acetonide precursor, followed by regioselective deprotection of the 3,5-isopropylidenyl group at the 3- and 5-positions using catalytic amount of an acid, followed by the benzylation of the 3- and 5-hydroxy groups to provide a compound of Formula XXI, is shown below.

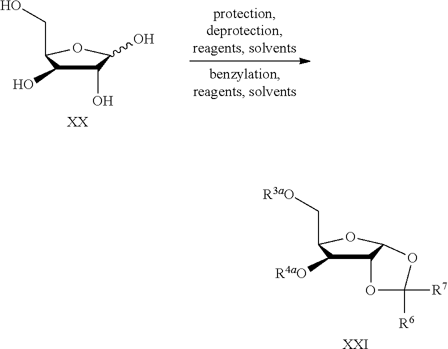

Where the ketal is an acetonide, precursor (i.e., R⁶ and R⁷ are methyl), the precursor to the ketal can be, for example, 2,2-dimethoxypropane, 2-methoxypropene, their derivatives, or/and acetone. Where the ketal or acetal includes other R⁶ and/or R⁷ groups, those of skill in the art can readily select an appropriate a ketone, aldehyde, ketal, acetal, or vinyl ether containing desired R⁶ and R⁷ groups to effect this conversion. While the R^{3a} and R^{4a} groups shown above are benzyl, other suitable protecting groups can be used.

In another aspect of this embodiment, β-D-ribose tetraacetate is reacted with trimethyl aluminum, for example, using the process disclosed in More and Camptell, Tetrahedron Letters, Volume 50, Issue 22, 3 Jun. 2009, Pages 2617-2619.

The acetate groups can then be hydrolyzed to form free hydroxyl groups, which can then be protected as a different protecting group (the reaction and subsequent deacetylation is shown below), or the acetate groups can be retained as protecting groups.

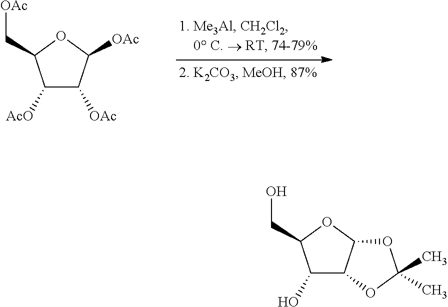

The 1,2-isopropylidenyl functional group on Formula XXI is removed by acid hydrolysis under conditions that do not cleave the protecting groups at the 3- and 5-positions. This is followed by regioselective oxidation of the 1-hydroxyl group to form a compound of Formula XXII.

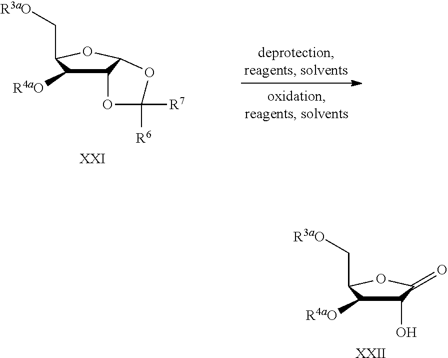

The 2-hydroxylactone of Formula XXII is then converted to the corresponding 2-chlorolactone of Formula XXIII using a substitution reaction. Some substitution reactions involve reacting the hydroxyl group itself, and others involve initially converting the hydroxyl group to a leaving group, such as tosylate, mesylate, triflate, brosylate, or nosylate. This substitution reaction inverts the stereochemistry at the 2-position.

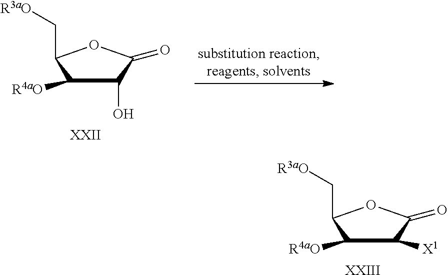

When the substitution reaction is carried out on the 2-hydroxyl group, representative reagents that can be used include CCl₄ and PPh₃ with or without imidazole (i.e., the Appel reaction), N-chlorosuccinimide (NCS) and PPh₃ (an Appel-type reaction), and sulfuryl chloride (SO₂Cl₂) and pyridine. When the substitution reaction is carried out by first converting the 2-hydroxyl group to a leaving group, representative reagents for converting the 2-hydroxyl group to a leaving group include trifluoromethanesulfonic anhydride (or TsCl, NsCl, MSCl) in the presence of a suitable base. The leaving group is then displaced with a source of chloride ions. Alternatively, the leaving group is displaced with a source of bromide or iodide ions.

One of the key reactions in this process is the conversion of the 2-chlorolactone of Formula XXIII to a 2-chloro, 2-fluorolactone of Formula XXIV. The chlorolactone is deprotonated at the 2-position using a strong, sterically-hindered base, then reacted with an electrophilic fluoride reagent, to provide 2-chloro-2-fluorolactone of Formula XXIV as a single diastereomer.

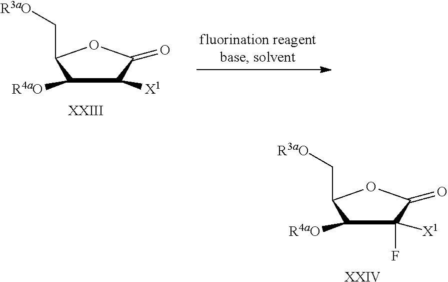

Where a source of bromide ions is used in place of chloride ions, the product is a 2-bromo-2-fluorolactone.

The 3- and 5-protecting groups are removed from the 2-chloro-2-fluorolactone of formula XXIV, and then two of the three free hydroxyl groups are regioselectively protected using a protecting group, such as a ketal or acetal, to provide an acyclic ester of formula XXV. The resulting 3- and 5-hydroxy groups are selectively protected to provide an acyclic ester of formula XXV.

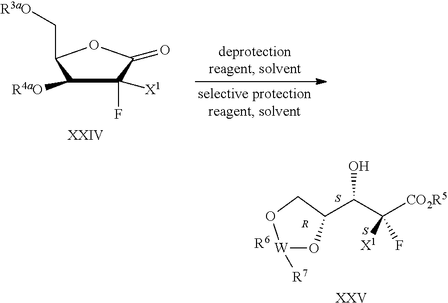

In the figure shown above, W can be carbon, boron, silicon, or phosphorus, with carbon being preferred, R⁶ or/and R⁷ can be hydrogen, C₁-₁₀ alkoxy, C₁-₁₀ alkyl, C₁-₁₀ branched alkyl, C₃-₆ cyclic alkyl, phenyl and benzyl, or aryl, and the isopropylidenyl protecting group (where W is C, R⁶ and R⁷ are methyl) is a preferred protecting group.

The chirality of the carbon to which the β(S)-hydroxyl group of the acyclic methyl ester of Formula XXV is attached can be converted by transforming the hydroxyl group into a leaving group, such as a sulfonyl ester, and then performing a substitution reaction to provide a corresponding β(R)-hydroxyl ester of Formula XXVI.

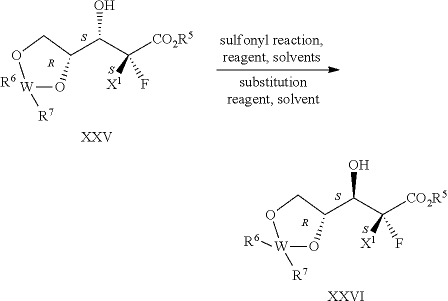

The unprotected hydroxyl group of the β(R)-hydroxyl ester of Formula XXVI is then protected, for example, with benzoyl chloride, and the —O—W(R⁵R⁶)O— protecting group, such as an isopropylidenyl group, is deprotected, for example, using acid hydrolysis. In the presence of an acid catalyst, the ester is converted to the corresponding lactone.

The remaining unprotected primary hydroxyl group can be protected, for example, with benzoyl chloride, to provide a cyclic dibenzoyl lactone of Formula I.

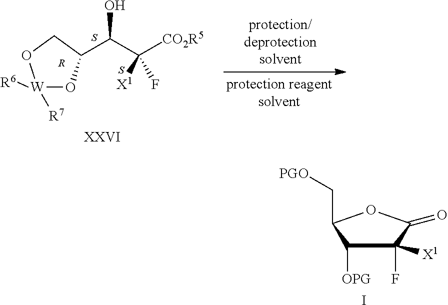

However, although benzoyl groups are exemplified above, either hydroxyl group can alternatively be protected with a different protecting group.

Once a compound of Formula I is prepared, it can be used as an intermediate to prepare a large number of antiviral and anticancer nucleosides and prodrugs thereof.

One way to do so is to selectively reduce the carbonyl moiety in the compounds of Formula I to a hydroxyl group, using reducing agents such as diisobutylaluminum hydride (DIBAL-H) or lithium tri-tert-butoxyaluminum hydride. The hydroxyl group can then be converted to a leaving group (LG), for example, by reacting the hydroxyl group with toluene sulfonyl chloride, in the presence of a base, to form a toluene sulfonate (tosylate) group.

Nucleoside compounds can be prepared by coupling the leaving group-substituted sugar with a protected, silylated or free natural or unnatural nucleoside base in the presence of Lewis acid such as TMSOTf. Deprotection of the 3′- and 5′-hydroxyls gives a nucleoside. Alternatively, nucleosides can be prepared from the leaving group-substituted sugar and a protected, silylated, or free natural or unnatural nucleoside base by performing a Mitsunobu reaction, and then deprotecting any protected functional groups. The resulting nucleosides can optionally be converted to monophosphate prodrugs.

Optionally, one or more positions on the sugar, base, and/or prodrug portion of the nucleosides, or on the intermediates described herein, other than the position including the X and F substitution, can be deuterated.

The present invention will be better understood with reference to the following Detailed Description.

### DETAILED DESCRIPTION

The present invention will be better understood with reference to the following definitions:

### I. Definitions

The term “independently” is used herein to indicate that the variable, which is independently applied, varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

As used herein, the term “enantiomerically pure” refers to a compound composition that comprises at least approximately 95%, and, preferably, approximately 97%, 98%, 99% or 100% of a single enantiomer of that compound.

As used herein, the term “substantially free of” or “substantially in the absence of” refers to a compound composition that includes at least 85 to 90% by weight, preferably 95% to 98% by weight, and, even more preferably, 99% to 100% by weight, of the designated enantiomer of that compound. In a preferred embodiment, the compounds described herein are substantially free of enantiomers.

Similarly, the term “isolated” refers to a compound composition that includes at least 85 to 90% by weight, preferably 95% to 98% by weight, and, even more preferably, 99% to 100% by weight, of the compound, the remainder comprising other chemical species or enantiomers.

The term “alkyl,” as used herein, unless otherwise specified, refers to a saturated straight, branched, or cyclic, primary, secondary, or tertiary hydrocarbons, including both substituted and unsubstituted alkyl groups. The alkyl group can be optionally substituted with any moiety that does not otherwise interfere with the reaction or that provides an improvement in the process, including but not limited to but limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included are CF₃ and CH₂CF₃.

In the text, whenever the term C(alkyl range) is used, the term independently includes each member of that class as if specifically and separately set out. The term “alkyl” includes C₁₋₂₂ alkyl moieties, and the term “lower alkyl” includes C₁₋₆ alkyl moieties. It is understood to those of ordinary skill in the art that the relevant alkyl radical is named by replacing the suffix “-ane” with the suffix “-yl”.

As used herein, a “bridged alkyl” refers to a bicyclo- or tricyclo alkane, for example, a 2:1:1 bicyclohexane.

As used herein, a “spiro alkyl” refers to two rings that are attached at a single (quaternary) carbon atom.

The term “alkenyl” refers to an unsaturated, hydrocarbon radical, linear or branched, in so much as it contains one or more double bonds. The alkenyl group disclosed herein can be optionally substituted with any moiety that does not adversely affect the reaction process, including but not limited to but not limited to those described for substituents on alkyl moieties. Non-limiting examples of alkenyl groups include ethylene, methylethylene, isopropylidene, 1,2-ethane-diyl, 1,1-ethane-diyl, 1,3-propane-diyl, 1,2-propane-diyl, 1,3-butane-diyl, and 1,4-butane-diyl.

The term “alkynyl” refers to an unsaturated, acyclic hydrocarbon radical, linear or branched, in so much as it contains one or more triple bonds. The alkynyl group can be optionally substituted with any moiety that does not adversely affect the reaction process, including but not limited to those described above for alkyl moeities. Non-limiting examples of suitable alkynyl groups include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, and hexyn-3-yl, 3,3-dimethylbutyn-1-yl radicals.

The term “alkylamino” or “arylamino” refers to an amino group that has one or two alkyl or aryl substituents, respectively.

The term “fatty alcohol” as used herein refers to straight-chain primary alcohols with between 4 and 26 carbons in the chain, preferably between 8 and 26 carbons in the chain, and most preferably, between 10 and 22 carbons in the chain. The precise chain length varies with the source. Representative fatty alcohols include lauryl, stearyl, and oleyl alcohols. They are colourless oily liquids (for smaller carbon numbers) or waxy solids, although impure samples may appear yellow. Fatty alcohols usually have an even number of carbon atoms and a single alcohol group (—OH) attached to the terminal carbon. Some are unsaturated and some are branched. They are widely used in industry. As with fatty acids, they are often referred to generically by the number of carbon atoms in the molecule, such as “a C12 alcohol”, that is an alcohol having 12 carbons, for example dodecanol.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis, and are described, for example, in Greene et al., Protective Groups in Organic Synthesis, supra.

The term “aryl”, alone or in combination, means a carbocyclic aromatic system containing one, two or three rings wherein such rings can be attached together in a pendent manner or can be fused. Non-limiting examples of aryl include phenyl, biphenyl, or naphthyl, or other aromatic groups that remain after the removal of a hydrogen from an aromatic ring. The term aryl includes both substituted and unsubstituted moieties. The aryl group can be optionally substituted with any moiety that does not adversely affect the process, including but not limited to but not limited to those described above for alkyl moieties. Non-limiting examples of substituted aryl include heteroarylamino, N-aryl-N-alkylamino, N-heteroarylamino-N-alkylamino, heteroaralkoxy, arylamino, aralkylamino, arylthio, monoarylamidosulfonyl, arylsulfonamido, diarylamidosulfonyl, monoaryl amidosulfonyl, arylsulfinyl, arylsulfonyl, heteroarylthio, heteroarylsulfinyl, heteroarylsulfonyl, aroyl, heteroaroyl, aralkanoyl, heteroaralkanoyl, hydroxyaralkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, and heteroarylalkenyl, carboaralkoxy.

The terms “alkaryl” or “alkylaryl” refer to an alkyl group with an aryl substituent. The terms “aralkyl” or “arylalkyl” refer to an aryl group with an alkyl substituent.

The term “halo,” as used herein, includes chloro, bromo, iodo and fluoro.

The term “acyl” refers to a carboxylic acid ester in which the non-carbonyl moiety of the ester group is selected from the group consisting of straight, branched, or cyclic alkyl or lower alkyl, alkoxyalkyl, including, but not limited to methoxymethyl, aralkyl, including, but not limited to, benzyl, aryloxyalkyl, such as phenoxymethyl, aryl, including, but not limited to, phenyl, optionally substituted with halogen (F, Cl, Br, or I), alkyl (including but not limited to C₁, C₂, C₃, and C₄) or alkoxy (including but not limited to C₁, C₂, C₃, and C₄), sulfonate esters such as alkyl or aralkyl sulphonyl including but not limited to methanesulfonyl, the mono, di or triphosphate ester, trityl or monomethoxytrityl, substituted benzyl, trialkylsilyl (e.g., dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The terms “alkoxy” and “alkoxyalkyl” embrace linear or branched oxy-containing radicals having alkyl moieties, such as methoxy radical. The term “alkoxyalkyl” also embraces alkyl radicals having one or more alkoxy radicals attached to the alkyl radical, that is, to form monoalkoxyalkyl and dialkoxyalkyl radicals. The “alkoxy” radicals can be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “heteroatom,” as used herein, refers to oxygen, sulfur, nitrogen and phosphorus.

The terms “heteroaryl” or “heteroaromatic,” as used herein, refer to an aromatic that includes at least one sulfur, oxygen, nitrogen or phosphorus in the aromatic ring.

The term “heterocyclic,” “heterocyclyl,” and cycloheteroalkyl refer to a nonaromatic cyclic group wherein there is at least one heteroatom, such as oxygen, sulfur, nitrogen, or phosphorus in the ring.

Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, and pteridinyl, aziridines, thiazole, isothiazole, 1,2,3-oxadiazole, thiazine, pyridine, pyrazine, piperazine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N⁶-alkylpurines, N⁶-benzylpurine, N⁶-halopurine, N⁶-vinypurine, N⁶-acetylenic purine, N⁶-acyl purine, N⁶-hydroxyalkyl purine, N⁶-thioalkyl purine, thymine, cytosine, 6-azapyrimidine, 2-mercaptopyrmidine, uracil, N⁵-alkylpyrimidines, N⁵-benzylpyrimidines, N⁵-halopyrimidines, N⁵-vinylpyrimidine, N⁵-acetylenic pyrimidine, N⁵-acyl pyrimidine, N⁵-hydroxyalkyl purine, and N⁶-thioalkyl purine, and isoxazolyl. The heteroaromatic group can be optionally substituted as described above for aryl. The heterocyclic or heteroaromatic group can be optionally substituted with one or more sub stituents selected from the group consisting of halogen, haloalkyl, alkyl, alkoxy, hydroxy, carboxyl derivatives, amido, amino, alkylamino, and dialkylamino. The heteroaromatic can be partially or totally hydrogenated as desired. As a nonlimiting example, dihydropyridine can be used in place of pyridine. Functional oxygen and nitrogen groups on the heterocyclic or heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenelsulfonyl. Substitution on trityl includes C₁₋₆ alkyl, C₁₋₆ oxyalkyl, halo, and aryl substitution on any of the phenyl groups of the trityl group. The heterocyclic or heteroaromatic group can be substituted with any moiety that does not adversely affect the reaction, including but not limited to but not limited to those described above for aryl.

The term “host,” as used herein, refers to a unicellular or multicellular organism in which the virus can replicate, including but not limited to cell lines and animals, and, preferably, humans. Alternatively, the host can be carrying a part of the viral genome, whose replication or function can be altered by the compounds of the present invention. The term host specifically refers to infected cells, cells transfected with all or part of the viral genome and animals, in particular, primates (including but not limited to chimpanzees) and humans. In most animal applications of the present invention, the host is a human being. Veterinary applications, in certain indications, however, are clearly contemplated by the present invention (such as for use in treating chimpanzees).

The term “peptide” refers to a natural or synthetic compound containing two to one hundred amino acids linked by the carboxyl group of one amino acid to the amino group of another.

The term “pharmaceutically acceptable salt or prodrug” is used throughout the specification to describe any pharmaceutically acceptable form (such as an ester) compound which, upon administration to a patient, provides the compound. Pharmaceutically-acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art.

Pharmaceutically acceptable prodrugs refer to a compound that is metabolized, for example hydrolyzed or oxidized, in the host to form the compound of the present invention. Typical examples of prodrugs include compounds that have biologically labile protecting groups on functional moieties of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. The prodrug forms of the compounds of this invention can possess antiviral activity, can be metabolized to form a compound that exhibits such activity, or both.

### II. Synthesis

In one embodiment, the present invention is directed to a process for making compounds of Formula I:

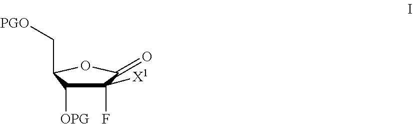

X¹ is selected from the group consisting of Cl, Br, and I.

PG is a hydroxyl protecting group. Representative hydroxyl protecting groups include, without limitation, tert-butyl dimethyl silyl, triisopropyl silyl, tert-butyl diphenyl silyl, benzyl, acyl, benzoyl, pivaloyl, trityl, substituted trityl and the like, with tent-butyl dimethyl silyl being a preferred embodiment.

Compounds of Formula I are useful intermediates for preparing antiviral nucleosides with F and X¹ substitution at the 2′-position. Such nucleosides are described, for example, in U.S. provisional applications 61/984,036, 62/073,937 and 62/155,939, international patent applications WO 2015/034420, WO 2015/056213 and WO 2015/081297, and U.S Publication No. 2015175648.

In one aspect of this embodiment, the method involves:

1) Reacting a 4-(S)-oxazolidinone such as Formula II with an X¹-substituted acetyl group to form an imide of Formula III.

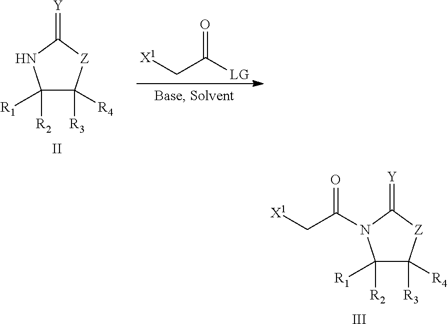

wherein:

Y is O or S,

Z is O, S, N-alkyl, N-aryl, N-alkylaryl, or N-arylalkyl;

R¹, R², R³ and R⁴ are, independently, selected from the group consisting of H, C₁₋₁₀ alkyl, C₁₋₁₀ branched alkyl, C₃₋₆ cycloalkyl, C₁₋₄(alkyl)aryl, benzyl, C₁₋₃(alkyl)C—O-aryl, CH₂OBz (where Bz=benzoyl), aryl, heteroaryl and C₃₋₆ heterocycloalkyl;

R¹ and R² or R³ and R⁴ can optionally be linked to form a C₃₋₈ cycloalkyl, alkyl substituted C₃₋₈ cycloalkyl or C₃₋₈ cycloalkyl fused to aryl;

LG is a leaving group, wherein representative leaving groups include, but are not limited to, halo, p-nitrophenol, imidazole, tosylate, brosylate, nosylate, mesylate, triflate, and the like, with halo being preferred;

Base is any base too hindered to displace the X¹ substituent on the X¹ substituted acetyl group, with representative bases including n-butyl lithium, lithium bis(trimethylsilyl)amide, lithium diisopropylamide, lithium tetramethylpiperidide, sec-butyl lithium, tert-butyl magnesium chloride, methyllithium, ethyllithium, 2-(ethylhexyl)lithium, isobutyllithium, isopropyllithium, tert-butyllithium, hexyllithium, sodium bis(trimethylsilyl)amide, potassium bis(trimethylsilyl)amide, lithium diethylamide, lithium dicyclohexylamide, lithium dimethylamide and the like with preference to n-butyl lithium.

The solvent is an organic solvent, which can be a polar aprotic solvent or a non-polar aprotic solvent, examples of which include methyltetrahydrofuran (2-Me-THF), dimethylether, methyl tert-butylether, methyl isobutylether, diglyme, chloroform, 1,2-dichloroethane, methylene chloride (dichloromethane/DCM), chlorobenzene, toluene, o-xylene, pentane, hexane, heptane, tetrahydrofuran (THF), dioxane, diethyl ether or mixtures of solvents thereof, with THF being preferred.

2) A compound of Formula III is allowed to react under basic conditions with (R)-2,2-disubstituted-1,3-dioxolane-4-carbaldehyde to provide a compound of Formula IV.

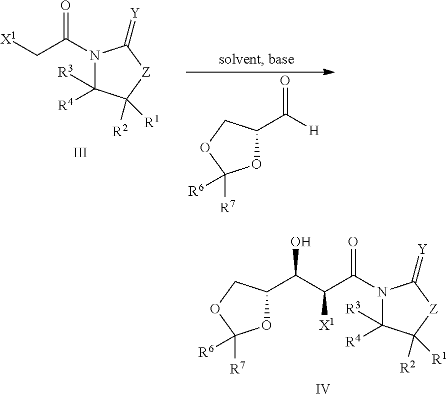

R⁶ and R⁷ are, independently, selected from the group consisting of C₁₋₁₀ alkyl, aryl, C₁₋₄ (alkyl)aryl, such as benzyl, heteroaryl and C₁₋₄ (alkyl)heteroaryl;

R⁶ and R⁷ optionally can be linked to form C₃₋₈ cycloalkyl, C₅₋₈ cycloheteroalkyl, alkyl substituted C₃₋₈ cycloalkyl and C₃₋₈ cycloalkyl fused to aryl,

The chirality of the dioxolate-4-carboxaldehyde and oxazolidinone drives the stereochemical preference in this reaction, which allows for the stereoselective fluorination in a later step.

Base is any base too sterically hindered to displace X¹, in Formula III, where representative bases include, but are not limited to, n-butyl lithium, lithium bis(trimethylsilyl)amide, lithium diisopropylamide, lithium tetramethylpiperidide, sec-butyl lithium, tert-butyl magnesium chloride, methyllithium, ethyllithium, 2-(ethylhexyl)lithium, isobutyllithium, isopropyllithium, tert-butyllithium, hexyllithium, sodium bis(trimethylsilyl)amide, potassium bis(trimethylsilyl)amide, lithium diethylamide, lithium dicyclohexylamide, lithium dimethylamide and the like with preference to lithium bis(trimethylsilyl)amide.

The reaction is preferably carried out in a polar, aprotic solvent, such as tetrahydrofuran (THF), dioxane or diethyl ether, with THF being preferred.

Alternatively, a compound of Formula III can be reacted under basic conditions, in the presence of a Lewis acid, with (R)-2,2-disubstituted-1,3-dioxolane-4-carbaldehyde to provide a compound of Formula IV (Eur. J. Org. Chem. 2015, 1314-1319, J. Org. Chem. 2012, 77, 543-555, Heterocycles 2007, 72, 339). Examples of such reagent combinations include (c-hexyl)₂BOTf, Et₃N, Bu₂BOTf, Et₃N (or lutidine) and 9-BBN-OTf, DIPEA, TiCl₄, TMEDA. Representative solvents include tetrahydrofuran, dichloromethane, dioxane, dichloroethane and the like, with dichloromethane being preferred.

3) The OH group in Compound IV is suitably protected with a protecting group (PG) which is largely stable to both basic conditions and acidic conditions, such as a silyl protecting group, which can be deprotected using a fluoride anion, or a group like benzyl, which can be deprotected using hydrogen, to form a compound of Formula V, typically in a polar, aprotic solvent such as tetrahydrofuran (THF), DCM, dioxane, diethyl ether and the like, with DCM being preferred.

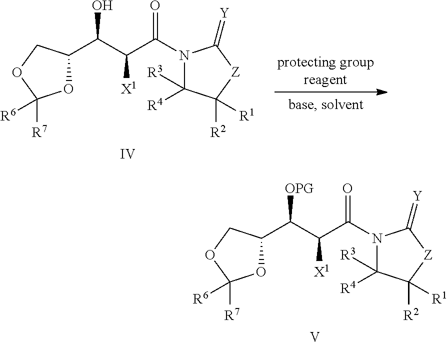

In this reaction, PG is a hydroxyl protecting group. Suitable hydroxyl protecting groups are well known to those skilled in the art, and include trimethylsilyl, dimethylhexylsilyl, t-butyldimethylsilyl, t-butyldiphenylsilyl, trityl or substituted trityl with preference to t-butyldimethylsilyl protection.

Examples of protecting group reagents which can react with a hydroxyl group, in the presence of a base, to form a hydroxyl protecting group PG include trimethylsilyl chloride, dimethylhexylsilyl chloride, t-butyldimethylsilyl chloride, t-butyldimethylsilyl triflate, t-butyldiphenylsilyl chloride, trityl chloride, tert-butyldiphenylsilyl triflate, benzyl triflate, Bn-chloroacetamidate, trimethylsilyl triflate. The formation of a protective group forms an acid, so a base is present to neutralize the acid. The base is one which does not displace X¹, and is typically a secondary or tertiary amine. Representative bases include pyridine, lutidines, triethylamine or diisopropyl ethyl amine, with 2,6-lutidine being preferred.

4) The oxazolidinone chiral auxiliary of compounds of Formula V is removed by reacting the compounds with an alkoxide of formula R⁵OM, where M is a metal, such as an alkali metal or alkaline earth metal, or a quaternary ammonium salt, and R⁵ is selected from the group consisting of C₁₋₁₀ alkyl, C₁₋₁₀ branched alkyl C₃₋₆ cycloalkyl and alkyl substituted C₃₋₈ cycloalkyl, to provide an ester of Formula VI.

Examples of these alcohol salts include sodium methoxide, lithium methoxide, potassium methoxide, magnesium methoxide, sodium ethoxide, lithium ethoxide, potassium ethoxide, magnesium ethoxide and sodium phenylmethanolate, with sodium methoxide being preferred. The reaction can be performed in the same alcohol (R⁵OH) as the above salt. Further, in place of, or in addition to the alcohol (R⁵OH), a polar aprotic solvent such as THF or dioxolane can be used. The reaction is shown below:

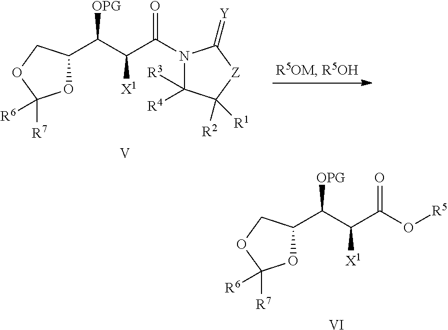

5) Selective introduction of fluorine represents a key step in the methods described herein, as the desired stereochemistry is obtained in high yield at what will be the 2 position of the desired ribonolactone. Compounds of Formula VI are converted to dihalo intermediates of formula VII under the action of a base, which also is too hindered to displace X¹, examples of which include n-butyl lithium, lithium bis(trimethylsilyl)amide, lithium diisopropylamide, lithium tetramethylpiperidide or sec-butyl lithium; lithium bis(trimethylsilyl)amide being preferred. The reaction is shown below:

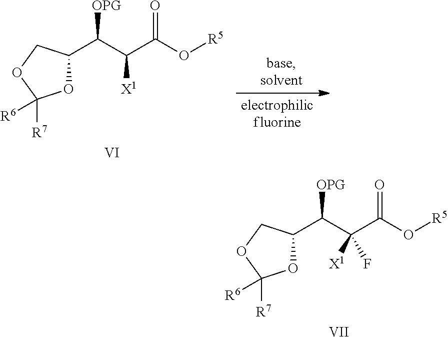

Examples of suitable solvents include polar and non-polar aprotic solvents, such as tetrahydrofuran (THF), dioxane, 2-methyltetrahydrofuran (2-Me-THF), dimethylether, methyl tert-butylether, methyl isobutylether, diglyme, chloroform, 1,2-dichloroethane, methylene chloride, chlorobenzene, toluene, o-xylene, acetone, acetonitrile, pentane, hexane, heptane and diethyl ether, with THF being preferred.

Any electrophilic source of fluorine can be used, examples of which include N-fluoro-o-benzenedisulfonimide (NFOBS), N-fluorobenzenesulfonimide (NFSI), 2-fluoro-3,3-dimethyl-2,3-dihydro-1,2-benzisothiazole 1,1-dioxide [124170-23-6], 1-fluoro-4-hydroxy-1,4-diazoniabicyclo[2,2,2]octane bis(tetrafluoroborate) on aluminum oxide (Accufluor), N-fluoropyridinium salts: [178439-26-4], [107264-00-96], [109705-14-8], [107264-09-5], [107263-95-6], [140623-89-8], [130433-68-0] and 1-chloromethyl-4-fluoro-1,4-diazoniabicyclo[2.2.2]octane bis(tetrafluoroborate) (Selectfluor), with NFSI being preferred.

6) Intermediates of Formula VII can be cyclized to form ribolactones of Formula VIII by the action of an acid, such as acetic acid, hydrochloric acid, trichloroacetic acid or sulfuric acid, with acetic acid being preferred, in a polar aprotic solvent such as THF, acetonitrile, or dioxane, with acetonitrile being preferred. The solvent may also contain water.

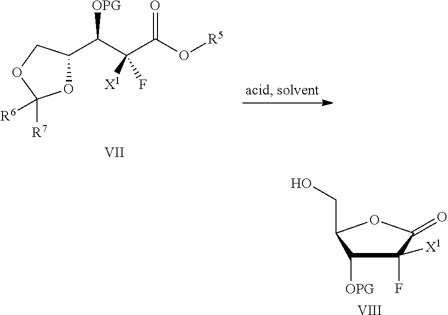

7) Finally, the free 5-alcohol group of VIII is protected by reaction with a protecting group reagent suitable for forming protecting groups PG, to give a ribonolactone of Formula I.

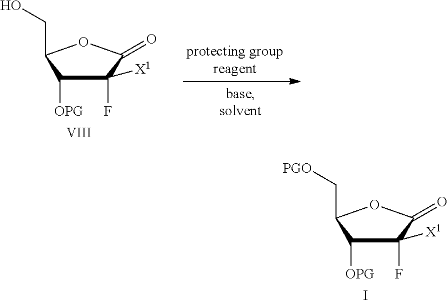

Suitable hydroxyl protecting groups are well known to those skilled in the art, and include trimethylsilyl, dimethylhexylsilyl, t-butyldimethylsilyl, t-butyldiphenylsilyl, benzyl, acyl, benzoyl, pivaloyl, trityl or substituted trityl, and selected alkyl groups, with preference to t-butyldimethylsilyl protection. As discussed above, the protecting groups are prepared by reacting the hydroxyl group with an appropriate protective group reagent capable of forming PG, in the presence of a base, such as a secondary or tertiary amine. Representative bases are those which can neutralize the acid formed while the protective group PG is formed, but which do not displace X¹, examples of which include pyridine, lutidines such as 2,6-lutidine, trimethylamine, triethylamine, and diisopropyl ethyl amine, with 2,6-lutidine being preferred.

An alternative approach involves a modified workup of the above step 2, which involves quenching the reaction that forms IV with an alcohol (R⁵OH) as defined above. Suitable protection of the secondary alcohol of the resulting ester IXa as outlined in the above in step 3 provides an intermediate of Formula VI, and this approach reduces one step in the overall process, while still providing desired compounds of Formula I. Some inconsequential scrambling of the carbon center to which X¹ is attached occurs during this step.

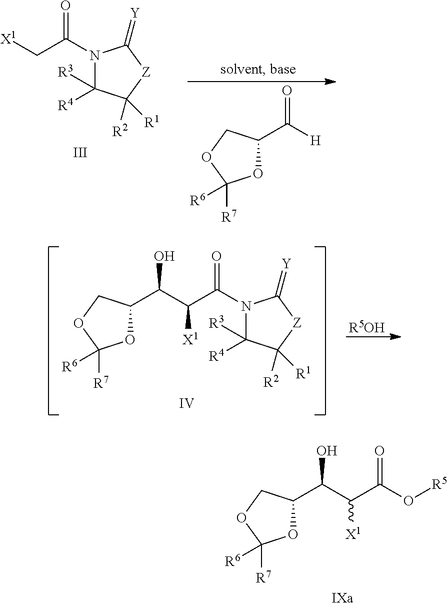

Method 2 involves the steps of:

1) Reacting an alpha halo acetate ester of Formula X with (R)-2,2-disubstituted-1,3-dioxolane-4-carbaldehyde in the presence of a base to provide compounds of Formula XI in which the 3-OH is predominately in the R stereochemical configuration.

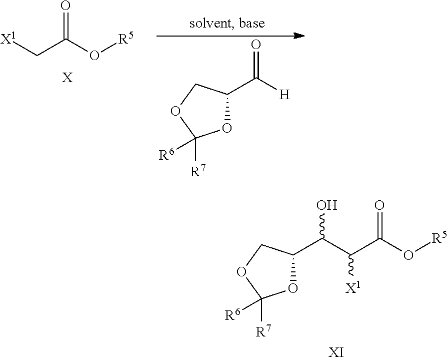

The base can be any base suitable for not displacing X¹, examples of which include n-butyl lithium, lithium bis(trimethylsilyl)amide, sodium bis(trimethylsilyl)amide, potassium bis(trimethylsilyl)amide, lithium diisopropylamide, lithium tetramethylpiperidide, sec-butyl lithium; with lithium diisopropylamide being preferred. The solvent can be any polar aprotic solvent, with representative solvents including tetrahydrofuran (THF), dioxane and diethyl ether, with THF being preferred.

An alternate procedure that can be used to prepare compounds of Formula XI in which the 3-OH is predominately in the R stereochemical configuration involves reacting compounds XII under the influence of metals [M] such as zinc or magnesium, or the action of boranes, including trialkylboranes such as triethylborane. The solvents for this reaction are typically polar aprotic solvents, such as tetrahydrofuran (THF), dioxane and diethyl ether, with THF being preferred.

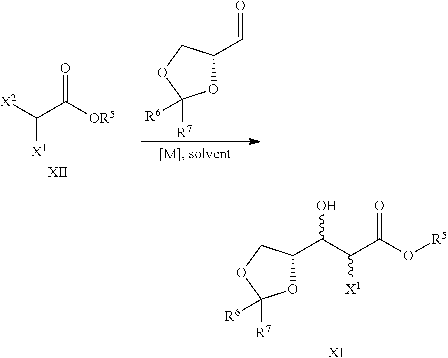

X² is a halogen selected from the group consisting of Cl, Br, and I, and X¹, R⁵, R⁶, and R⁷ are as described above.

2) The free hydroxyl group on the compounds of Formula XI can be protected as PG using a protection group reagent in the presence of a base, such as a secondary or tertiary amine, in a suitable polar, aprotic solvent such as THF, DCM, acetonitrile, or dioxane, with DCM being preferred and results in XIII. Suitable alcohol protecting groups are well known to those skilled in the art, and include trimethylsilyl, dimethylhexylsilyl, t-butyldimethylsilyl, t-butyldiphenylsilyl, benzyl, trityl or substituted trityl and selected alkyl groups with preference to t-butyldimethylsilyl protection. Examplary bases include pyridine, lutidines such as 2,6-lutidine, triethylamine, and diisopropyl ethyl amine, with 2,6-lutidine being preferred. The protected XI can now be separated into diastereomers XIII

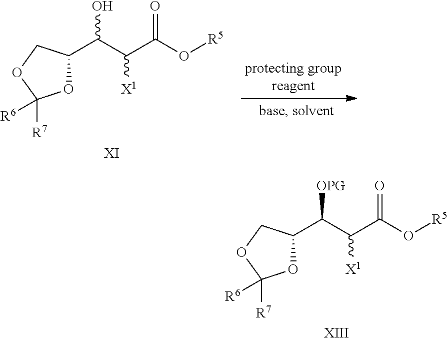

In this Scheme, R⁵, R⁶, R⁷, X¹ and PG are as defined in the earlier schemes.

3) Selective introduction of fluorine represents a key step in this art as the desired stereochemistry is obtained in high yield at what will be the 2 position of the desired ribolactone. Compounds of Formula XIII are converted to dihalo intermediates of Formula XIV under the action of a base which is too hindered to displace X¹, examples include n-butyl lithium, lithium bis(trimethylsilyl)amide, sodium bis(trimethylsilyl)amide, potassium bis(trimethylsilyl)amide, lithium diisopropylamide, lithium tetramethylpiperidide and sec-butyl lithium; with lithium bis(trimethylsilyl)amide being preferred.

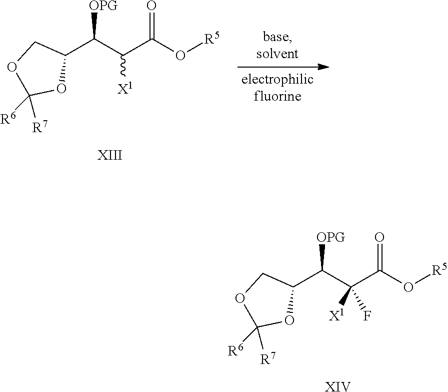

The reaction is typically carried out in a polar, aprotic solvent, such as tetrahydrofuran (THF), dioxane or diethyl ether, with THF being preferred. The electrophilic fluorine source is defined in step 5 above.

4) Compounds of Formula XIV can be converted to ribofuranoses of Formula I as outlined above in Method 1.

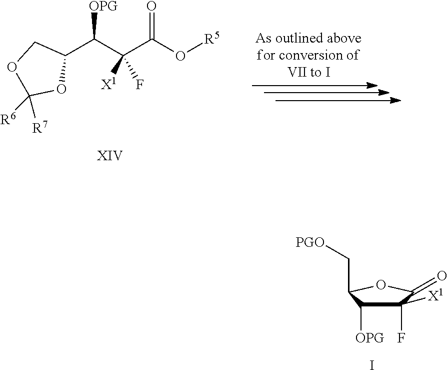

Method 3 includes the following steps:

Another procedure that can be used to prepare compounds of Formula I involves the following steps.

1) Oxidation of compounds XV with an oxidizing reagent followed by condensation with an α,β-unsaturated ester formation reagent to form compound XVI. Oxidizing agent is a inorganic or organic oxidizing reagent that include, but are not limited to, NaIO₄, HIO₄, KIO₄, LiIO₄, Pb(OAc)₄, NaBiO₃, CrO₂Cl₂, PhI(OAc)₂, with NaIO₄ being preferred; The base can be an inorganic or organic base that includes NaHCO₃, KHCO₃, Na₂CO₃, K₂CO₃, 1,8-diazabicyclo[5.4.0]undec-7-ene (DBU), triethylamine, with NaHCO₃ and K₂CO₃ being preferred; The α, β-unsaturated ester formation reagent includes alkyl trialkylphosphonoacetate and (carbalkoxymethylene)triarylphosphorane. The reaction is carried out with a suitable solvent. Suitable solvents include a polar, aprotic solvent or a mixed solvent, such as water, acetone, tetrahydrofuran (THF), dioxane, 2-methyltetrahydrofuran (2-Me-THF), methyl tert-butylether, methyl isobutylether, diglyme, chloroform, 1,2-dichloroethane, methylene chloride, acetonitrile, with water being preferred. (Tetrahedron 1989, 45(2), 391-402).

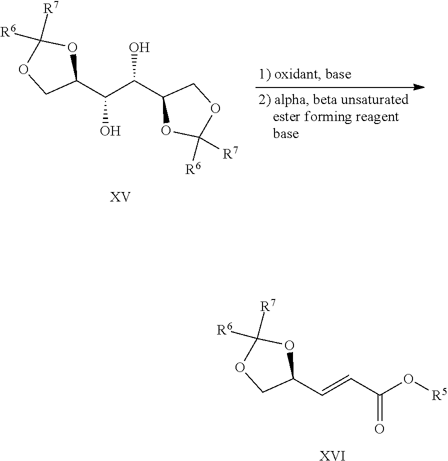

2) The carbon-carbon double bond can be converted to dihydroxyl groups by reacting with a dihydroxylation reagent. The dihydroxylation agent is an inorganic or organic oxidizing reagent that includes, but are not limited to, AD-mix-β, OsO₄, Pb(OAc)₄, NaMnO₄, KMnO₄, with AD-mix-β being preferred; The reaction is carried out in a polar, aprotic solvent or mixed solvent, such as water, 1-butanol, 2-butanol, tert-butanol, acetone, tetrahydrofuran (THF), dioxane, 2-methyltetrahydrofuran (2-Me-THF), diethylether, methyl tert-butylether, methyl isobutylether, diglyme, chloroform, 1,2-dichloroethane, methylene chloride, acetonitrile, with water and tert-butanol being preferred. (Tetrahedron 1994, 50(31), 9457-9470).

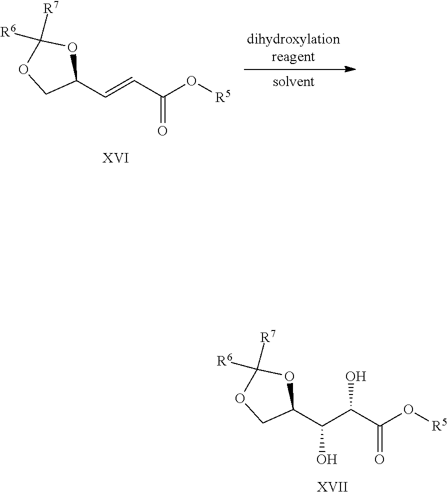

3) The α-hydroxyl group can be selectively converted into a leaving group by reaction with a reagent that forms a leaving group, forming compound XVIII. Leaving group reagents include, but are not limited to, methanesulfonyl chloride, benzenesulfonyl chloride, 4-substituted benzenesulfonyl, 4-nitrobenzenesulfonyl chloride, 4-chlorobenezenesulfonyl chloride, 4-bromobenezenesulfonyl chloride, p-toluenesulfonyl chloride; triflic anhydride, trifluoromethanesulfonyl chloride with preference to 4-nitrobenzenesulfonyl chloride. Base is a base that is suitable for neutralizing the acid that is formed during the step of converting the hydroxyl group to a protecting group, while not displacing X¹, examples of which include organic or inorganic bases such as 1-methylimidazole, pyridine, lutidines, DBU and 1,4-diazabicyclo[2.2.2]octane (DABCO); N,N,N′,N′-tetramethylethylenediamine (TMEDA), sodium bicarbonate, potassium carbonate, and cesium carbonate with preference to pyridine. The reaction is carried out in a polar, aprotic solvent or mixed solvent, such as dichloromethane, tetrahydrofuran (THF), acetonitrile, dioxane, diethyl ether and dichloromethane/water or diethyl ether/water. (Tetrahedron 1994, 50(31), 9457-9470).

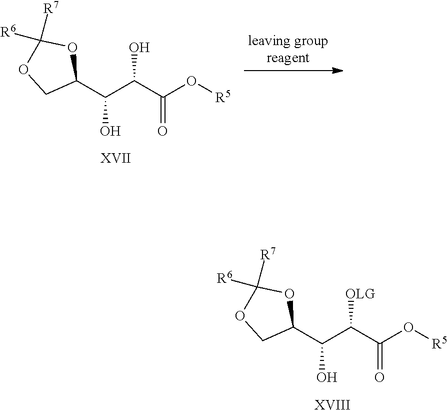

4) The leaving group of compound XVIII can be replaced by a halogen using halogen delivery reagents, to form a compound of Formula XIX. Halogenation delivery reagents RX¹ are inorganic or organic salts, in which X¹ includes Cl, Br and I, and R is Li, Na, K, or NBu₄, with LiCl and LiBr being preferred. The reaction is carried out in a polar, aprotic solvent or mixed solvent, such as DMF, acetonitrile, acetone, tetrahydrofuran (THF), dioxane, 2-methyltetrahydrofuran (2-Me-THF), diethylether, methyl tert-butylether, methyl isobutylether, diglyme, chloroform, 1,2-dichloroethane, methylene chloride with preference to DMF. (Tetrahedron 1994, 50(31), 9457-9470). In one embodiment, a crown ether, such as 18-crown-6, is added to accelerate the rate of reaction.

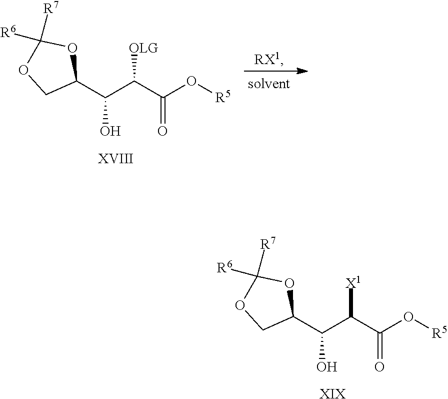

5) Compounds of Formula XIX can be converted to ribofuranoses of Formula I by the same methods as outlined above for the conversion of XI to I.

In an alternative embodiment, rather than protecting the hydroxyl moiety in XIX after the nucleophilic displacement, the hydroxyl moiety in XVIII can be protected, and then the molecule subjected to nucleophilic displacement.

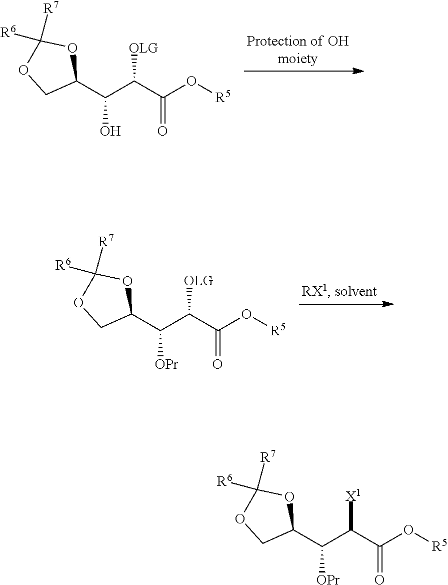

Method 4 includes the following steps:
1) This method initially involves forming a furanose intermediate in which the 1-hydroxy and 2-hydroxy groups are protected as a ketal or acetal, and the 3- and 5-hydroxy groups are protected as a different type of protecting group, such that the ketal can be hydrolyzed in the presence of the protecting group on the 3- and 5-hydroxy groups. The typical conditions for forming a ketal or acetal with the 1- and 2-hydroxy groups also tend to form a ketal or acetal with the 3- and 5-hydroxy groups. Alternatively, a similarly protected compound is selected as the starting material. The latter may be preferred if such compounds are commercially available.

D-xylose is a preferred monosaccharide, and is the exemplified compound shown below as Compound XX. Where the ketal is formed with acetone, it is an acetonide, though other ketals or acetals can also be used. One of skill in the art can readily select an appropriate starting material to prepare a ketal or acetal that includes R⁶ and R⁷ groups, as such starting materials are typically ketones or aldehydes, ketals or acetals, or vinyl ethers, which include these R⁶ and R⁷ groups.

Once the 1- and 2-hydroxyl groups are protected, the hydroxyl groups at the 3- and 5-positions are protected, for example, as benzyl groups. Benzylation can take place, for example, by reacting the ketal-protected compound with a benzyl halide in the presence of a base.

The selective protection of the 1- and 2-hydroxyl groups on furanoses such as D-xylose (Formula XX) with an acetonide precursor, followed by regioselective deprotection of the 3,5-isopropylidenyl group at the 3- and 5-positions using catalytic amount of an acid, followed by the benzylation of the 3- and 5-hydroxy groups using benzyl chloride in the presence of a base to provide a compound of Formula XXI, is shown below.

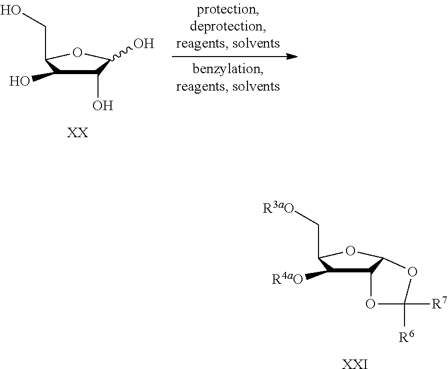

Where the ketal is an acetonide, (i.e., R⁶ and R⁷ are methyl), the precursor to the ketal can be, for example, 2,2-dimethoxypropane, 2-methoxypropene, their derivatives, or/and acetone. Where the ketal or acetal includes other R⁶ and/or R⁷ groups, those of skill in the art can readily select an appropriate a ketone, aldehyde, ketal, acetal, or vinyl ether containing desired R⁶ and R⁷ groups to effect this conversion. The acid catalyst can beselected from a list including H₂SO₄, HClO₄, TsOH, pyridinium p-toluenesufonate (PPTS), or HCl, optionally including a copper salt such as CuSO₄ (Bioorg. Med. Chem. 2006, 14, 500-509).

While the R^{3a} and R^{4a} groups are preferably benzyl, other suitable protecting groups include, but are not limited to, benzyl derivatives such as 4-halobenzyl, 4-methylbenzyl, 4-methoxybenzyl, 4-nitrobenzyl, 2,4-dimethylbenzyl groups (Bioorg. Med. Chem. 2012, 20, 6321-6334). The base used in the benzylation reaction can be any suitable base, examples of which include sodium hydride, potassium hydride, potassium hydroxide, sodium hydroxide, cesium hydroxide, sodium carbonate, potassium carbonate and cesium carbonate with or without phase transfer catalysts, including tetra-butylammonium bromide, tetra-butylammonium iodide, tetra-butylammonium chloride, tetra-benzylammonium halides, tetra-butylammonium hydroxide. The solvent can be any aprotic or/and protic solvent, with representative solvents including N,N-dimethylformamide (DMF), tetrahydrofuran (THF), 1,4-dioxane, acetonitrile, acetone, dichloromethane, hexane, water, ethanol and methanol.

An alternate procedure involves protecting all four hydroxyl groups of D-xylose using 2,2′-dimethoxypropane, or 2-methoxypropene in acetone in the presence of catalytic amount of H₂SO₄, HClO₄, or HCl, optionally along with CuSO₄, followed by selective deprotection of the 3,5-acetonide group with a catalytic amount of an acid strong enough to hydrolyze the 3,5-acetonide group. A representative acid is 0.5% aqueous H₂SO₄ solution of the 3- and 5-hydroxyl groups are then reacted with benzyl bromide in the presence of a base such as NaH in an aprotic solvent such as DMF (method A), or a base such as KOH and n-Bu₄NI in an aprotic solvent such as CH₂Cl₂, with catalytic water (method B) to provide a compound of formula XXI. As with the other procedure, the hydroxyl groups can be protected as other ketals or acetals, and other protecting groups than benzyl can be used to protect the 3- and 5-hydroxy groups.

In another aspect of this embodiment, β-D-ribose tetraacetate is reacted with trimethyl aluminum, for example, using the process disclosed in More and Camptell, Tetrahedron Letters, Volume 50, Issue 22, 3 Jun. 2009, Pages 2617-2619.

The acetate groups can then be hydrolyzed to form free hydroxyl groups, which can then be protected as a different protecting group (the reaction and subsequent deacetylation is shown below), or the acetate groups can be retained as protecting groups.

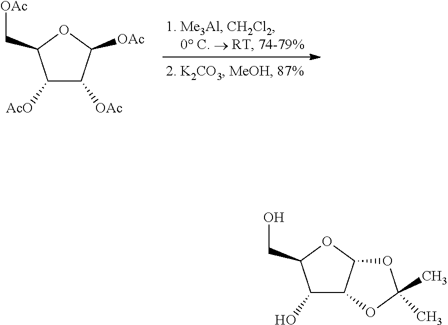

2) The 1,2-isopropylidenyl functional group on a compound of formula XXI is removed by acid hydrolysis under conditions that do not cleave the protecting groups at the 3- and 5-positions. This is followed by regioselective oxidation of the 1-hydroxyl group to form a compound of formula XXII.

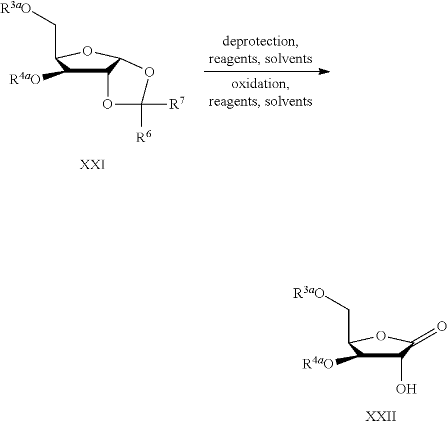

Examples of acids which can be used for the acid hydrolysis step include acetic acid, formic acid, trifluoroacetic acid (TFA), trichloroacetic acid (TCA), TsOH, pyridinium p-toluenesufonate (PPTS), camphorsulfonic acid, and H₂SO₄ (Biochemistry 2009, 48, 10882-10893; Tetrahedron 2008, 64, 11686-11696). The solvent is typically water but could also include solvents such as low molecular weight alcohols. Representative oxidation methods include, but are not limited to, using a combination of N-iodosuccinimide (NIS) and tetra-n-butylammonium iodide (n-Bu₄NI) (see Fujioka et al., Tetrahedron Letters, Volume 51, Issue 15, 14 Apr. 2010, Pages 1945-1946), bromine (Br₂) and potassium percarbonate (K₂CO₃₎ (or BaCO₃ or CaCO₃), RhH(PPh₃)₄, or Ag₂CO₃ (Tetrahedron 1998, 54, 13591-13620; Org. Process Res. Dev. 2005, 9, 457-465; Org. Lett. 2014, 16, 3384-3387).

The solvent for the oxidation step can be any aprotic or/and protic solvents, with representative solvents including N,N-dimethylformamide (DMF), tetrahydrofuran (THF), 1.4-dioxane, acetonitrile, dichloromethane, chloroform, water, methanol, ethanol, and isopropyl alcohol.
3) The 2-hydroxylactone of formula XXII is then converted to the corresponding 2-chlorolactone of formula XXIII Some substitution reactions involve reacting the hydroxyl group itself, and others involve initially converting the hydroxyl group to a leaving group, such as tosylate, mesylate, triflate, brosylate, or nosylate.

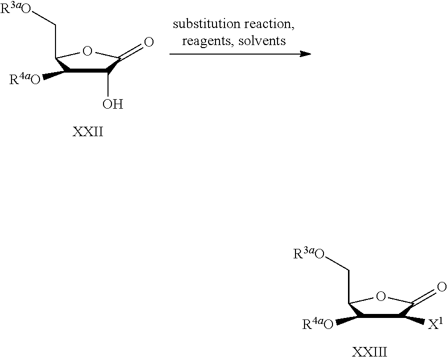

When the substitution reaction is carried out on the 2-hydroxyl group, representative reagents that can be used include CCl₄ and PPh₃ with or without imidazole (i.e., the Appel reaction), N-chlorosuccinimide (NCS) and PPh₃ (an Appel-type reaction), and sulfuryl chloride (SO₂Cl₂) and pyridine with preference to sulfuryl chloride (SO₂Cl₂) and pyridine. When the substitution reaction is carried out by first converting the 2-hydroxyl group to a leaving group, representative reagents for converting the 2-hydroxyl group to a leaving group include trifluoromethanesulfonic anhydride (or TsCl, NsCl, MSCl) in the presence of a suitable base such as pyridine. In the case of X¹═Br preference is to trifluoromethanesulfonic anhydride and pyridine. The leaving group is then displaced with a source of chloride ion, such as tetra-alkylammonium chloride or LiCl. Alternatively, the leaving group is then displaced with a source of bromide ion, such as tetra-alkylammonium bromide or LiBr. In the case of X¹═Br, preference is to LiBr. (Angew. Chem. Int. Ed. 1975, 14, 801-811; J. Org. Chem. 1998, 63, 7472-7480). Representative bases include pyridine, triethylamine (Et₃N), N,N-diisopropylamine (DIEA), and 4-(dimethylamino)pyridine (DMAP). The solvent is typically an aprotic solvents, such as N,N-dimethylformamide (DMF), tetrahydrofuran (THF), 1,4-dioxane, acetonitrile, dichloromethane, 1,2-dichloroethane or chloroform with preference to dichloromethane.
4) One of the key reactions in this process is the conversion of the 2-chlorolactone of formula XXIII to a 2-chloro, 2-fluorolactone of formula XXIV. The chlorolactone is reacted with an electrophilic fluoride reagent in the presence of a strong, sterically-hindered base to provide 2-chloro-2-fluorolactone of formula XXIV as a single diastereomer.

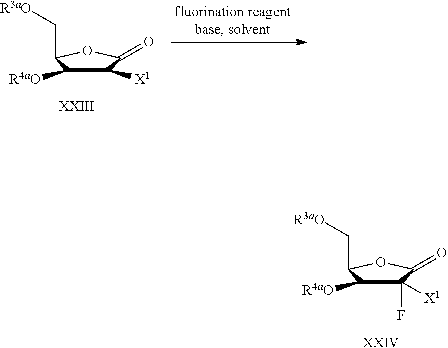

Representative electrophilic fluoride reagents include, but are not limited to, N-fluorobenzenesulfonimide (NFSI), 1-fluoro-2,4,6-trimethylpyridinium tetrafluoroborate, 1-fluoropyridinium tetrafluoroborate, 1-fluoropyridinium triflate, and 1-chloromethyl-4-fluoro-1,4-diazoniabicyclo[2,2,2]octane bis(tetrafluoroborate) (F-TEDA, selectfluor® fluorinating reagent) with preference to NFSI (J. Org. Chem. 1998, 63, 2161-2167; J. Org. Chem. 2009, 74, 5779-5789. The base can be any base suitable for deprotonating alpha to the carbonyl on the 2-chlorolactone, examples of which include lithium hydride, sodium hydride, potassium hydride, n-butyl lithium, sec-butyl lithium, lithium bis(trimethylsilyl)amide, lithium diisopropylamide, lithium tetramethylpiperidide; with lithium bis(trimethylsilyl)amide being preferred. The solvent can be any polar aprotic solvent, with representative solvents including tetrahydrofuran (THF), dioxane and diethyl ether, with THF being preferred.
5) The 3- and 5-protecting groups are removed from the 2-chloro-2-fluorolactone of formula XXIV, and the two of the three free hydroxyl groups are then regioselectively protected using a protecting group, such as a ketal or acetal, to provide an acyclic ester of formula XXV. Where the protecting groups at the 3- and 5-positions are benzyl groups, these groups can be removed by hydrogenation with or without a catalyst in alcoholic solvent which also ring opens the lactone to form an ester. The resulting 3- and 5-hydroxy groups are selectively protected to provide an acyclic ester of formula XXV.

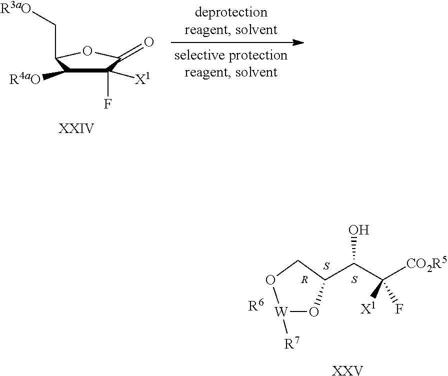

In the figure shown above, W can be carbon, boron, silicon, or phosphorus, with carbon being preferred, R⁶ or/and R⁷ can be hydrogen, C₁-₁₀ alkoxy, C₁-₁₀ alkyl, C₁-₁₀ branched alkyl, C₃-₆ cyclic alkyl, phenyl and benzyl, or aryl, and the isopropylidenyl protecting group (where W is C, R⁶ and R⁷ are methyl) is a preferred protecting group.

The hydrogenolysis of the 2-chloro-2-fluorolactone of formula XXIV can be performed with Pd/C under H₂ (1 atm) in an alcoholic solvent such as methanol or ethanol, or catalytic hydrogen transfer (Pd/C, 1,4-cyclohexadiene) in a suitable solvent.

Where the ketal or acetal group is an acetonide group, it can be introduced by using 2,2-dimethoxypropane, 2-methoxypropene, or/and acetone in the presence of catalytic amount of acid, such as H₂SO₄, HClO₄, TsOH, or HCl, optionally including CuSO₄. Any suitable solvent can be used, with representative solvents including N,N-dimethylformamide (DMF), tetrahydrofuran (THF), 1,4-dixoxane, acetone, methanol, ethanol, and isopropyl alcohol, with methanol being preferred.

6) The chirality of the carbon to which the β(S)-hydroxyl group of the acyclic ester of formula XXV is attached can be converted by transforming the hydroxyl group into a leaving group, such as a sulfonyl ester, and then performing a substitution reaction to provide a corresponding β(R)-hydroxyl ester of formula XXVI.

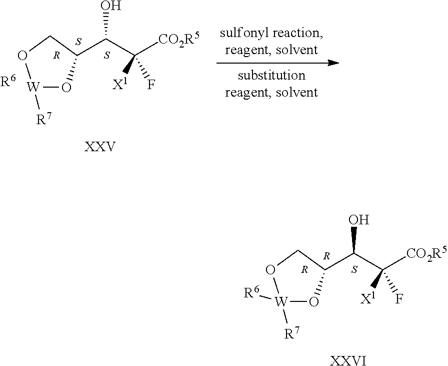

Representative sulfonyl esters include triflate (Tf), nosylate (Ns), tosylate (Ts), and meslate (Ms), though other leaving groups can be prepared as well.

Examples of suitable reagents which can be used to displace the sulfonyl ester include potassium nitrite, cesium propionate, cesium benzoate, lithium benzoate, potassium benzoate, tetra-alkylammonium benzoate, cesium trifluoroacetate, lithium trifluoroacetate, and cesium acetate (Synlett. 1997, 1077-1078; J. Org. Chem. 1987, 52, 4230-4234; Tetrahedron 1998, 54, 14487-14514; J. Chem. Soc. Perkin Trans. 1, 2002, 1982-1998), with potassium nitrite being preferred. The reaction is optionally carried out in the presence of a crown ether such as 18-crown-6, and/or activated molecular sieves (Eur. J. Org. Chem. 2001, 473-476; J. Med. Chem. 2004, 47, 4570-4587; J. Am. Chem. Soc. 2007, 129, 11892-11893). The solvent can be any aprotic solvent, with representative solvents including N,N-dimethylformamide (DMF), tetrahydrofuran (THF), and 1,4-dioxane, with DMF being preferred.

One specific set of conditions which can be used involves reacting the β(R)-hydroxyl group of an acyclic ester of formula XXV with trifluorosulfonic anhydride and pyridine in CH₂Cl₂ at −20° C., which successfully affords a triflate ester, which is then treated with potassium nitrite in the presence of 18-crown-6 and activated 3 A molecular sieves in DMF at room temperature to provide its corresponding R-configuration ester of formula XXVI.
7) The unprotected hydroxyl group of the β(R)-hydroxyl ester of formula XXVI is protected, for example, with benzoyl chloride, and the —O—W(R⁶R⁷)O— protecting group, such as an isopropylidenyl group, is deprotected, for example, using acid hydrolysis. In the presence of an acid catalyst, the ester is converted to the corresponding lactone. The remaining unprotected primary hydroxyl group can be protected, for example, with benzoyl chloride, to provide a cyclic diprotected lactone of formula I.

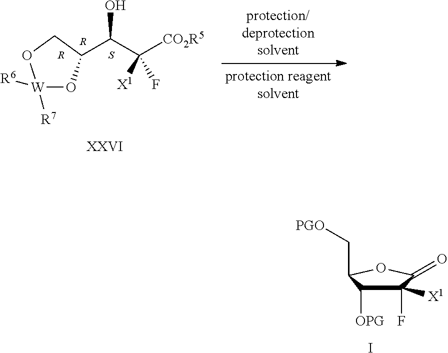

However, although benzoyl groups are exemplified above, either hydroxyl group can alternatively be protected with a different protecting group.

The protecting group (PG) can be derived from commercially available benzoyl chlorides/bromides and anhydrides, with benzoyl chloride being preferred. The removal conditions for the isopropylidenyl group can be an acidic conditions suitable for cleavage of acetonide function, examples of which include acetic acid, formic acid, trifluoroacetic acid (TFA), trichloroacetic acid (TCA), TsOH, pyridinium p-toluenesulfonate (PPTS), camphorsulfonic acid analogs and H₂SO₄, with TFA being preferred. The protection of a hydroxyl group (PG) may require the presence of base such as Et₃N, DIPEA, DMAP, and pyridine. The solvent can be any aprotic or/and protic solvents, with representative solvents including N,N-dimethylformamide (DMF), tetrahydrofuran (THF), 1,4-dixoxane, dichloromethane, 1,2-dichloroethane, chloroform, acetonitrile, toluene, chlorobezene, H₂O, methanol, ethanol, isopropyl alcohol, with dichloromethane, acetonitrile, or toluene being preferred.

Methods for the facile preparation of active nucleoside and nucleoside phosphoramidate compounds are known in the art and result from the selective combination known methods. The compounds disclosed herein can be prepared as described in detail below, or by other methods known to those skilled in the art. It will be understood by one of ordinary skill in the art that variations of detail can be made without departing from the spirit and in no way limiting the scope of the present invention.

The various reaction schemes are summarized below.
Scheme 1 is a non-limiting example of the synthesis of active compounds of the present invention, and in particular, a synthetic approach to nucleosides XXVIII.
Scheme 2 is a non-limiting example of the synthesis of active compounds of the present invention, and in particular, an alternate synthetic approach to nucleosides XXVIII.
Scheme 3 is a non-limiting example of the synthesis of active compounds of the present invention, and in particular, a synthetic approach to monophosphate prodrugs XXXV.
Scheme 4 is a non-limiting example of the synthesis of active compounds of the present invention, and in particular, a synthetic approach to monophosphate prodrugs XXXIX, XXXX and XXXXI.
Scheme 5 is a non-limiting example of the synthesis of active compounds of the present invention, and in particular, a synthetic approach to monophosphate prodrugs XXXXIV.
Scheme 6 is a non-limiting example of the synthesis of active compounds of the present invention, and in particular, a synthetic approach to monophosphate prodrugs XXXXVII.

The compounds of Formula I can be converted to active nucleosides and prodrugs thereof, using methods known to those of ordinary skill in the art, for example, those methods outlined in: (a) Rajagopalan, P.; Boudinot, F. D; Chu, C. K.; Tennant, B. C.; Baldwin, B. H.; Antiviral Nucleosides: Chiral Synthesis and Chemotheraphy: Chu, C. K.; Eds. Elsevier: 2003. b) Recent Advances in Nucleosides: Chemistry and Chemotherapy: Chu, C. K.; Eds. Elsevier: 2002. c) Frontiers in Nucleosides & Nucleic Acids, 2004, Eds. R. F. Schinazi & D. C. Liotta, IHL Press, Tucker, Ga., USA, pp: 319-37 d) Handbook of Nucleoside Synthesis: Vorbruggen H. & Ruh-Pohlenz C. John Wiley & sons 2001), and by general Schemes 1-2.

Specifically, the carbonyl moiety in the compounds of Formula I can be selectively reduced to a hydroxyl group by reagents such as diisobutylaluminum hydride (DIBAL-H) or lithium tri-tert-butoxyaluminum hydride, and the hydroxyl group can then be converted to a leaving group (LG). For example, reaction of the hydroxyl group with toluenesulfonyl chloride, in the presence of a base, can convert the hydroxyl group to a toluene sulfonate (tosylate) group such as XXVII (Scheme 1). Other known reactions can convert the hydroxyl group to a different leaving group, such as a halogen, benzoate or acetate (XXVII). From there, one can follow Schemes 1-6 as shown below to provide 2′-dihalo nucleosides and prodrugs thereof.

As shown below in Scheme 1, compound XXVII can be prepared by coupling sugar XXVII with a protected, silylated or free natural or unnatural nucleoside base in the presence of Lewis acid such as TMSOTf. Deprotection of the 3′- and 5′-hydroxyls gives nucleoside XXVIII.

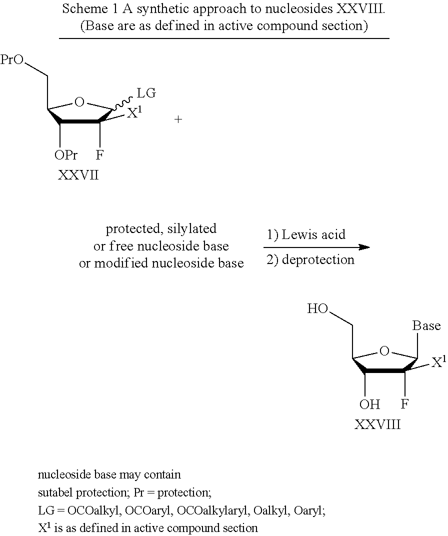

In the schemes described herein, if a nucleoside base includes functional groups that might interfere with, or be decomposed or otherwise converted during the coupling steps, such functional groups can be protected using suitable protecting groups. After the coupling step, protected functional groups, if any, can be deprotected.

Alternatively, nucleosides XXVIII can be prepared from 1-halo, 1-sulfonate or 1-hydroxy compounds XXIX. For the case of 1-halo or 1-sulfonate a protected or free nucleoside base in the presence of a base such as triethyl amine or sodium hydride followed by deprotection provides nucleosides XXVIII. For the case of 1-hydroxy a protected or free nucleoside base in the presence of a Mitsunobu coupling agent such as diisopropyl azodicarboxylate followed by deprotection provides nucleosides XXVIII.

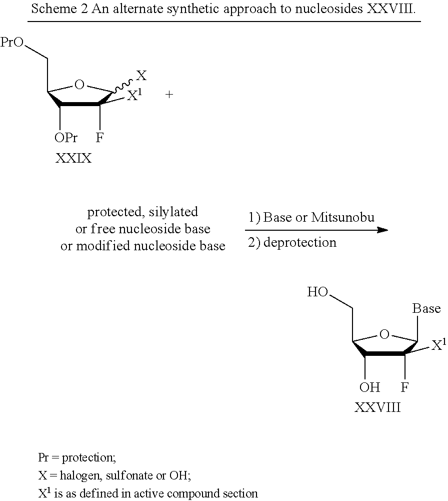

In the case of C-nucleosides prepared from bases:

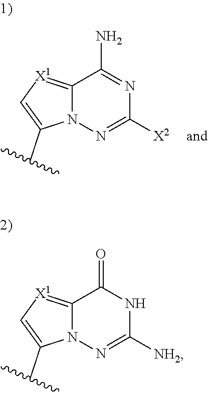

methods outlined in WO09132123, WO09132135, WO2011150288 and WO2011035250 can be used.

Monophosphate prodrugs XXXV can be prepared as outlined in Scheme 3 starting from phenol XXX. Exposure of XXX to phosphorous oxychloride or phosphorothioyl trichloride provides XXXI, which is subsequently allowed to react with an amino ester XXXII to give phosphoramidate XXXIII. R¹⁷ is defined as a natural or unnatural amino acid side chain. Nucleoside XXVIII can next be converted to monophosphate analog XXXIV by reaction of the 5′-hydroxyl group with the chlorophosphorylamino propanoate, XXXIII. Removal of protecting groups from the base and/or sugar, if present, provides monophosphate prodrugs XXXV.

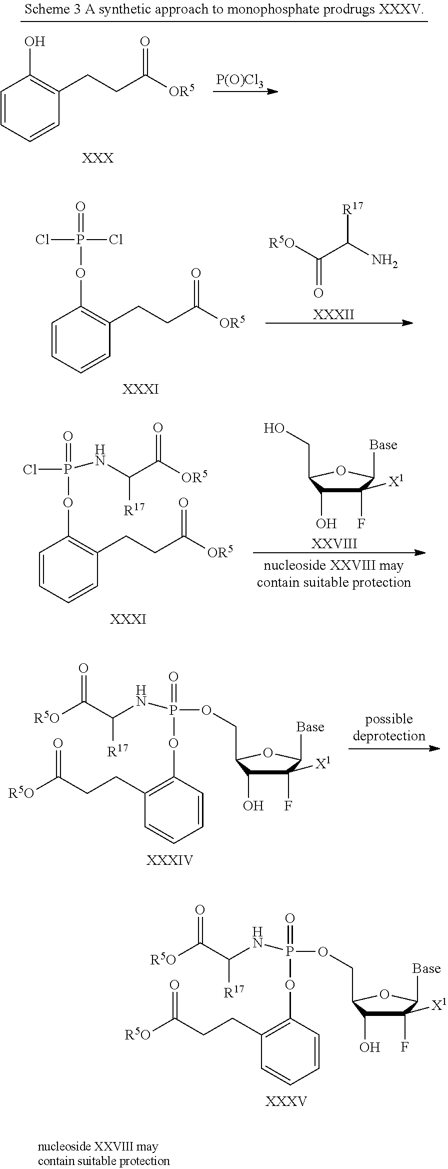

Monophosphate prodrugs XXXIX can be prepared by reaction of substituted pyridine XXXVI with phosphorous oxychloride. R²⁰ is selected from NH, N-alkyl, N-aryl, N-alkylaryl, N-arylalky, O, or S. The resulting intermediate can next be reacted with an ester of an L-amino acid XXXII (Scheme 4) to give XXXVIII. Nucleoside XXVIII can next be converted to monophosphate analog XXXIX by reaction of the 5′-hydroxyl group with the chlorophosphoryl substrate, XXXVIII. Removal of protecting groups, if necessary, provides monophosphate prodrugs XXXIX.

Utilizing a similar protocol with substitution of XXXII by R¹⁵OH or XXXVI, monophosphate prodrugs XXXX and XXXXI can also be prepared. R¹⁵ selected from the group consisting of H, Li, Na, K, C₁₋₂₀alkyl, C₃₋₆cycloalkyl, C₁₋₄(alkyl)aryl, benzyl, C₁₋₆haloalkyl, C₂₋₃(alkyl)OC₁₋₂₀alkyl, aryl, and heteroaryl, wherein aryl includes phenyl and heteroaryl includes pyridinyl, and wherein phenyl and pyridinyl are optionally substituted with zero to three substituents independently selected from the group consisting of (CH₂)₀₋₆CO₂R¹⁶ and (CH₂)₀₋₆ CON(R¹⁶)₂.

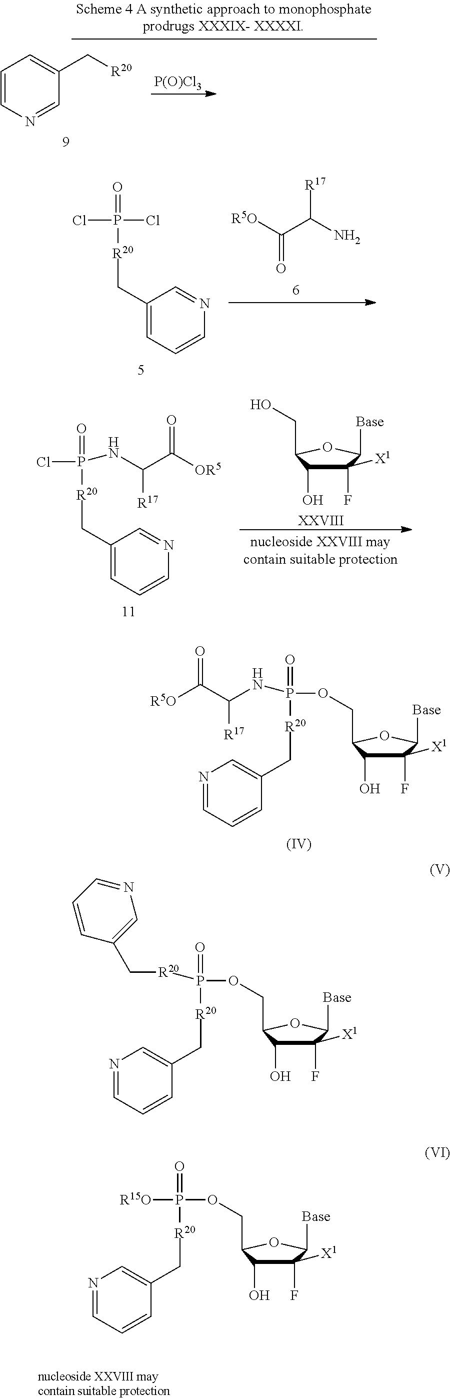

Monophosphate prodrugs XXXXIV can be prepared by reaction of XXXXII with phosphorous oxychloride to give XXXXIII (Scheme 5). Nucleoside XXVIII can next be converted to monophosphate analog XXXXIV by reaction of the 5′-hydroxyl group with the chlorophosphoryl substrate, XXXXIII. Removal of protecting groups, if necessary, provides monophosphate prodrugs XXXXIV.

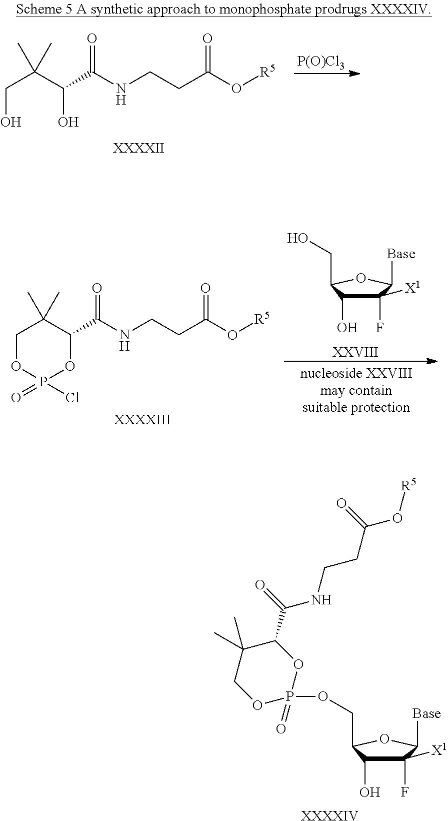

Monophosphate prodrugs XXXXVII can be prepared by reaction of XXXXV with phosphorous oxychloride to give XXXXVI (Scheme 6). R²¹ is selected from the group consisting of H, C₁₋₂₀ alkyl, C₁₋₂₀ alkenyl, the carbon chain derived from a fatty acid, and C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, heteroaryl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃-₁₀ cycloalkyl, or cycloalkyl. Nucleoside XXVIII can next be converted to monophosphate analog XXXXVII by reaction of the 5′-hydroxyl group with the chlorophosphoryl substrate, XXXXVI. Removal of protecting groups, if necessary, provides monophosphate prodrugs XXXXVII.

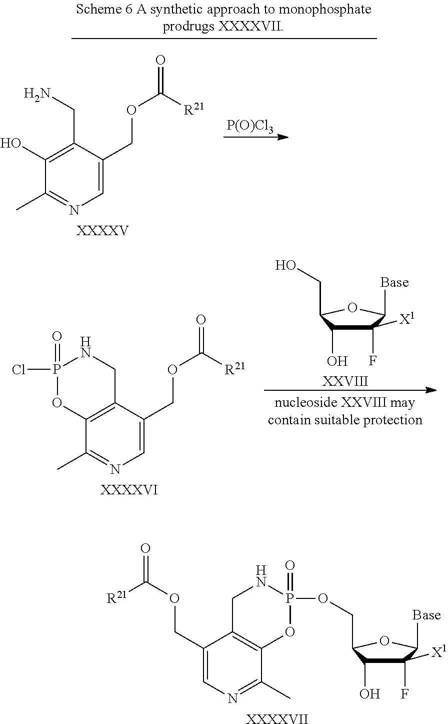

Incorporation of Deuterium:

It is expected that single or multiple replacement of hydrogen with deuterium (carbon-hydrogen bonds to carbon-deuterium bond) at site(s) of metabolism in the sugar portion of a nucleoside antiviral agent will slow down the rate of metabolism. This can provide a relatively longer half-life, and slower clearance from the body. The slow metabolism of a therapeutic nucleoside is expected to add extra advantage to a therapeutic candidate, while other physical or biochemical properties are not affected. Intracellular hydrolysis or deuterium exchanges my result in liberation of deuterium oxide (D₂O).

Methods for incorporating deuterium into amino acids, phenol, sugars, and bases, are well known to those of skill in the art. Representative methods are disclosed in U.S. Pat. No. 9,045,521.

A large variety of enzymatic and chemical methods have been developed for deuterium incorporation at both the sugar and nucleoside stages to provide high levels of deuterium incorporation (D/H ratio). The enzymatic method of deuterium exchange generally has low levels of incorporation. Enzymatic incorporation has further complications due to cumbersome isolation techniques which are required for isolation of deuterated mononucleotide blocks. Schmidt et al., Ann. Chem. 1974, 1856; Schmidt et al., Chem. Ber., 1968, 101, 590, describes synthesis of 5′,5′-²H₂-adenosine which was prepared from 2′,3′-O-isopropylideneadenosine-5′-carboxylic acid or from methyl-2,3-isopropylidene-beta-D-ribofuranosiduronic acid, Dupre, M. and Gaudemer, A., Tetrahedron Lett. 1978, 2783. Kintanar, et al., Am. Chem. Soc. 1998, 110, 6367 reported that diastereoisomeric mixtures of 5′-deuterioadenosine and 5′(R/S)-deuteratedthymidine can be obtained with reduction of the appropriate 5′-aldehydes using sodium borodeuteride or lithium aluminum deuteride (98 atom % ²H incorporation). Berger et al., Nucleoside & Nucleotides 1987, 6, 395 described the conversion of the 5′-aldehyde derivative of 2′-deoxyguanosine to 5′- or 4′-deuterio-2′-deoxyguanosine by heating the aldehyde in ²H₂O/pyridine mixture (1:1) followed by reduction of the aldehyde with NaBD₄.

Ajmera et al., Labelled Compd. 1986, 23, 963 described procedures to obtain 4′-deuterium labeled uridine and thymidine (98 atom % ²H). Sinhababu, et al., J. Am. Chem. Soc. 1985, 107, 7628) demonstrated deuterium incorporation at the C3′ (97 atom % ²H) of adenosine during sugar synthesis upon stereoselective reduction of 1,2:5,6-di-O-isopropylidene-β-D-hexofuranos-3-ulose to 1,2:5,6-di-O-isopropylidene-3-deuterio-β-D-ribohexofuranose using sodium borodeuteride and subsequently proceeding further to the nucleoside synthesis. Robins, et al., Org. Chem. 1990, 55, 410 reported synthesis of more than 95% atom ²H incorporation at C3′ of adenosine with virtually complete stereoselectivity upon reduction of the 2′-O-tert-butyldimethylsilyl(TBDMS) 3-ketonucleoside by sodium borodeuteride in acetic acid. David, S. and Eustache, J., Carbohyd. Res. 1971, 16, 46 and David, S. and Eustache, J., Carbohyd. Res. 1971, 20, 319 described syntheses of 2′-deoxy-2′(S)-deuterio-uridine and cytidine. The synthesis was carried out by the use of 1-methyl-2-deoxy-2′-(S)-deuterio ribofuranoside.

Radatus, et al., J. Am. Chem. Soc. 1971, 93, 3086 described chemical procedures for synthesizing 2′-monodeuterated (R or S)-2′-deoxycytidines. These structures were synthesized from selective 2-monodeuterated-2-deoxy-D-riboses, which were obtained upon stereospecific reduction of a 2,3-dehydro-hexopyranose with lithium aluminum deuteride and oxidation of the resulting glycal. Wong et al. J. Am. Chem. Soc. 1978, 100, 3548 reported obtaining deoxy-1-deuterio-D-erythro-pentose, 2-deoxy-2(S)-deuterio-D-erythro-pentose and 2-deoxy-1,2(S)-dideuterio-D-erythro-pentose from D-arabinose by a reaction sequence involving the formation and LiAlD₄ reduction of ketene dithioacetal derivatives.

Pathak et al. J., Tetrahedron 1986, 42, 5427) reported stereospecific synthesis of all eight 2′ or 2′-deuterio-2′-deoxynucleosides by reductive opening of appropriate methyl 2,3-anhydro-beta-D-ribo or beta-D-lyxofuranosides with LiAlD₄. Wu et al. J. Tetrahedron 1987, 43, 2355 described the synthesis of all 2′,2′-dideuterio-2′-deoxynucleosides, for both deoxy and ribonucleosides, starting with oxidation of C2′ of sugar and subsequent reduction with NaBD₄ or LiAlD₄ followed by deoxygenation by tributyltin deuteride. Roy et al. J. Am. Chem. Soc. 1986, 108, 1675, reported 2′,2′-dideuterio-2′-deoxyguanosine and thymidine can be prepared from 2-deoxyribose 5-phosphate using 2-deoxyribose 5-phosphate aldolase enzyme in ²H₂O achieving some 90 atom % deuteration. Similarly, the synthesis of 4′,5′,5′-²H₃-guanosine can be carried out.

Therefore, it is clear that each position of the sugar residue can be selectively labeled.

A useful alternative method of stereospecific deuteration was developed to synthesize polydeuterated sugars. This method employed exchange of hydrogen with deuterium at the hydroxyl bearing carbon (i.e. methylene and methine protons of hydroxyl bearing carbon) using deuterated Raney nickel catalyst in ²H₂O.

Various techniques are available to synthesize fully deuterated deoxy and ribonucleosides. Thus in one method, exchange reaction of deuterated Raney nickel-²H₂O with sugars, a number of deuterated nucleosides specifically labeled at 2′, 3′ and 4′ positions were prepared. The procedure consisted of deuteration at 2′, 3′ and 4′ positions of methyl beta-D-arabinopyranoside by Raney nickel-²H₂O exchange reaction followed by reductive elimination of 2′-hydroxyl group by tributyltin deuteride to give methyl beta-D-2′,2′,3′,4′-²H₄-2-deoxyribopyranoside, which was converted to methyl beta-D-2′,2′,3′,4′-²H₄-2′-deoxyribofuranoside and glycosylated to give various 2′,2′,3′,4′-²H₄-nucleosides (>97 atom % ²H incorporation for H3′ & H4′.

The synthesis of deuterated phenols is described, for example, in Hoyer, H. (1950), Synthese des pan-Deutero-o-nitro-phenols. Chem. Ber., 83: 131-136. This chemistry can be adapted to prepare substituted phenols with deuterium labels. Deuterated phenols, and substituted analogs thereof, can be used, for example, to prepare phenoxy groups in phosphoramidate prodrugs.

The synthesis of deuterated amino acids is described, for example, in Matthews et al., Biochimica et Biophysica Acta (BBA)—General Subjects, Volume 497, Issue 1, 29 Mar. 1977, Pages 1-13. These and similar techniques can be used to prepare deuterated amino acids, which can be used to prepare phosphoramidate prodrugs of the nucleosides described herein.

One method for synthesizing a deuterated analog of the compounds described herein involves synthesizing a deuterated ribofuranoside with 2′-fluoro, 2′-chloro substitution; and attaching a nucleobase to the deuterated ribofuranoside to form a deuterated nucleoside. A prodrug, such as a phosphoramidate prodrug, can be formed by modifying the 5′-OH group on the nucleoside. Where a deuterated phenol and/or deuterated amino acid is used, one can prepare a deuterated phosphoramidate prodrug.

Another method involves synthesizing a ribofuranoside with 2′-fluoro, 2′-chloro substitution, and attaching a deuterated nucleobase to form a deuterated nucleoside. This method can optionally be performed using a deuterated furanoside to provide additional deuteration. As with the method described above, the nucleoside can be converted into a prodrug form, which prodrug form can optionally include additional deuteration.

A third method involves synthesizing a ribofuranoside with 2′-fluoro, 2′-chloro substitution, attaching a nucleobase to form a nucleoside, and converting the nucleoside to a phosphoramidate prodrug using one or both of a deuterated amino acid or phenol analog in the phosphoramidate synthesis.

Accordingly, using the techniques described above, one can provide one or more deuterium atoms in the sugar, base, and/or prodrug portion of the nucleoside compounds described herein.

### III Stereoisomerism and Polymorphism

The compounds described herein can have asymmetric centers and occur as racemates, racemic mixtures, individual diastereomers or enantiomers, with all isomeric forms being included in the present invention. Compounds of the present invention having a chiral center can exist in and be isolated in optically active and racemic forms. Some compounds can exhibit polymorphism. The present invention encompasses racemic, optically-active, polymorphic, or stereoisomeric forms, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. One can either purify the respective compound, then derivatize the compound to form the compounds described herein, or purify the compound themselves.

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.

i) physical separation of crystals: a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization: a technique whereby the individual enantiomers are separately crystallized from a solution of the racemate, possible only if the latter is a conglomerate in the solid state;

iii) enzymatic resolutions: a technique whereby partial or complete separation of a racemate by virtue of differing rates of reaction for the enantiomers with an enzyme;

iv) enzymatic asymmetric synthesis: a synthetic technique whereby at least one step of the synthesis uses an enzymatic reaction to obtain an enantiomerically pure or enriched synthetic precursor of the desired enantiomer;

v) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;

vi) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors: a synthetic technique whereby the desired enantiomer is obtained from non-chiral starting materials and where the stereochemical integrity is not or is only minimally compromised over the course of the synthesis;

x) chiral liquid chromatography: a technique whereby the enantiomers of a racemate are separated in a liquid mobile phase by virtue of their differing interactions with a stationary phase (including but not limited to via chiral HPLC). The stationary phase can be made of chiral material or the mobile phase can contain an additional chiral material to provoke the differing interactions;

xi) chiral gas chromatography: a technique whereby the racemate is volatilized and enantiomers are separated by virtue of their differing interactions in the gaseous mobile phase with a column containing a fixed non-racemic chiral adsorbent phase;

xii) extraction with chiral solvents: a technique whereby the enantiomers are separated by virtue of preferential dissolution of one enantiomer into a particular chiral solvent;

xiii) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available.

The terms used in describing the invention are commonly used and known to those skilled in the art. As used herein, the following abbreviations have the indicated meanings:
ACN acetonitrile
aq aqueous
BSA Bis(trimethylsilyl)acetamide
BzCl Benzoyl chloride
CDI carbonyldiimidazole
DIPEA diisopropyl ethyl amine (Hünig's base)
DMF N,N-dimethylformamide
DMSO dimethylsulfoxide
EDC 1-ethyl-3-(3-dimethyllaminopropyl) carbodiimide hydrochloride
EtOAc ethyl acetate
h hour
HOBt N-hydroxybenzotriazole
LiHMDS Lithium Hexamethyldisilazide
M molar
min minute
Ms mesylate
NCS N-chlorosuccinimide
NBS N-bromosuccinimide
NFSI N-fluorobenzenesulfonimide
NIS N-iodosuccinimide
NMI 1-Methylimidazole
Pyr pyridine
rt or RT room temperature
TBDPSCl tert-Butyl(chloro)diphenylsilane
TBAF Tetrabutylammonium fluoride
TBAT tetrabutylammonium triphenyldifluorosilicate
TBTU O-Benzotriazol-1-yl)-N,N,N′,N′-tetramethyluronium tetrafluoroborate
TEA triethylamine
THF tetrahydrofuran
Ts tosylate
TBSCl

### IX. General Methods for Preparing Active Compounds

It will be understood by one of ordinary skill in the art that variations of detail can be made without departing from the spirit and in no way limiting the scope of the present invention.

### SPECIFIC EXAMPLES

Specific compounds which are representative of this invention were prepared as per the following examples and reaction sequences; the examples and the diagrams depicting the reaction sequences are offered by way of illustration, to aid in the understanding of the invention and should not be construed to limit in any way the invention set forth in the claims which follow thereafter. The present compounds can also be used as intermediates in subsequent examples to produce additional compounds of the present invention. No attempt has necessarily been made to optimize the yields obtained in any of the reactions. One skilled in the art would know how to increase such yields through routine variations in reaction times, temperatures, solvents and/or reagents.

Anhydrous solvents were purchased from Aldrich Chemical Company, Inc. (Milwaukee, Wis.) and EMD Chemicals Inc. (Gibbstown, N.J.). Reagents were purchased from commercial sources. Unless noted otherwise, the materials used in the examples were obtained from readily available commercial suppliers or synthesized by standard methods known to one skilled in the art of chemical synthesis. Melting points (mp) were determined on an Electrothermal digit melting point apparatus and are uncorrected. ¹H and ¹³C NMR spectra were taken on a Varian Unity Plus 400 spectrometer at room temperature and reported in ppm downfield from internal tetramethylsilane. Deuterium exchange, decoupling experiments or 2D-COSY were performed to confirm proton assignments. Signal multiplicities are represented by s (singlet), d (doublet), dd (doublet of doublets), t (triplet), q (quadruplet), br (broad), bs (broad singlet), m (multiplet). All J- values are in Hz. Mass spectra were determined on a Micromass Platform LC spectrometer using electrospray techniques. Elemental analyses were performed by Atlantic Microlab Inc. (Norcross, Ga.). Analytic TLC was performed on Whatman LK6F silica gel plates, and preparative TLC on Whatman PK5F silica gel plates. Column chromatography was carried out on Silica Gel or via reverse-phase high performance liquid chromatography.

### Example 1

### Method 1 2-Cl Up, 2-F Down

### Synthesis of 2-Deoxy-2-fluoro-2-chloro-3,5-di-O-(tert-butyldimethylsilyl)-D-ribono-lactone

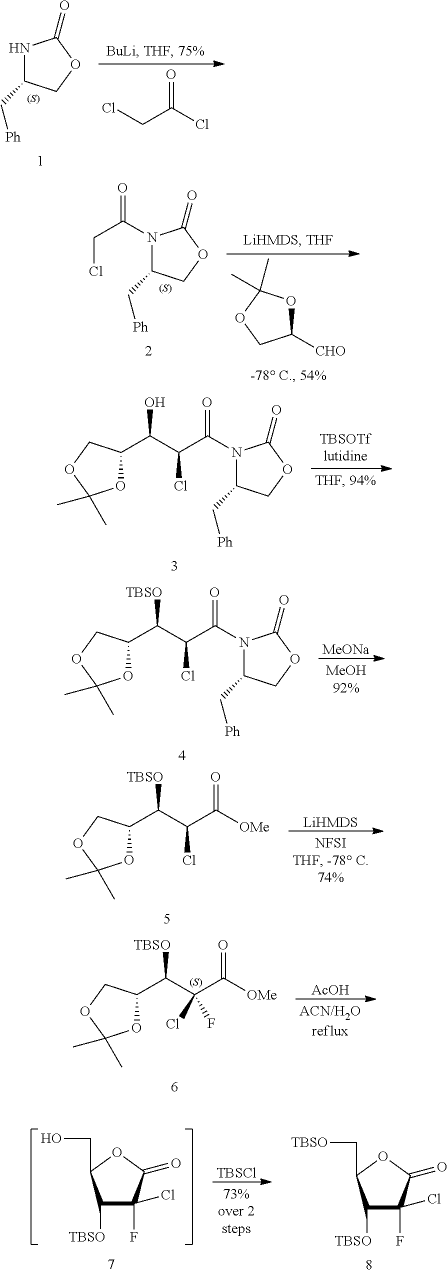

### (S)-4-benzyl-3-(2-chloroacetyl)oxazolidin-2-one (2)

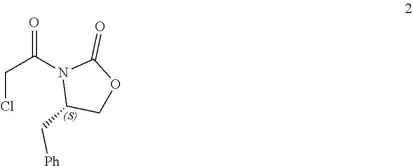

To a solution of (S)-4-Benzyl-2-oxazolidinone, 1 (5 g, 28.2 mmol) in dry THF (150 mL) at −78° C. under N₂ was added BuLi (2.5 M in hexane, 13.5 mL, 33.9 mmol). The solution was stirred at −78° C. for 15 min. and chloroacetyl chloride (2.47 mL, 31.0 mmol) was added in one portion. After full conversion was observed (typically 30 min.), the reaction was quenched by addition of a saturated aqueous solution of NH₄Cl and allowed to reach room temperature. The mixture was diluted with EtOAc, washed with water and brine. The organic layer was dried over MgSO₄ and concentrated under reduced pressure. The remainder was purified over silica gel column chromatography to afford a colorless syrup, which crystalized after standing to afford a white solid of 2 after filtration (5.1 g, 71%). Alternatively, precipitation can be initiated by dilution of the syrup in cold diethyl ether to afford a white solid after filtration.

### (S)-4-benzyl-3-((2S,3R)-2-chloro-3-((R)-2,2-dimethyl-1,3-dioxolan-4-yl)-3-hydroxypropanoyl)oxazolidin-2-one (3)

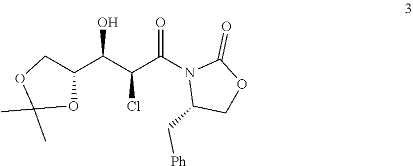 [SMILES: CC1(C)OC[C@H]([C@@H](O)[C@H](Cl)C(=O)N2C(=O)OC[C@@H]2Cc2ccccc2)O1]

### Procedure Using LDA:

To a solution of diisopropylamine (152 μL, 1.08 mmol) in dry THF (5 mL) at −78° C. under N₂ was added nBuLi (2.5 M in hexane, 433 μL, 1.08 mmol). After 15 min., a THF (2 mL) solution of 2 (250 mg, 0.985 mmol) was added dropwise via cannula at −78° C. The mixture was stirred 30 min at this temperature and a THF (2 mL) solution of freshly distilled (R)-2,2-dimethyl-1,3-dioxolane-4-carbaldehyde (115 μL, 1.28 mmol) was added dropwise via cannula. After 30 minutes stirring, the reaction was quenched at −78° C. by addition of 1M HCl. The mixture was rapidly worked up by dilution with EtOAc followed by washings with water and brine. The organic layer was dried over MgSO₄ and filtered on a silica pad prior to concentration under reduced pressure. The remainder was purified over silica gel column chromatography (Hex/EA 95:5 to 70:30) to afford the desired aldol product 3 as a single diastereomer in 44% yield.

### Procedure Using LiHMDS:

To a solution of LiHMDS (1 M in THF, 1.48 mL, 1.48 mmol) in dry THF (5 mL) at −78° C. under N₂ was added dropwise via cannula a solution of 2 (250 mg, 0.985 mmol) in THF (2 mL). The mixture was stirred 30 min at this temperature and a solution of freshly distilled (R)-2,2-dimethyl-1,3-dioxolane-4-carbaldehyde (115 μL, 1.28 mmol) in THF (mL) was added dropwise via cannula. After 30 minutes stirring, the reaction was quenched at −78° C. by addition of 1M HCl. The mixture was rapidly worked up by dilution with EtOAc followed by washings with water and brine. The organic layer was dried over MgSO₄ and filtered on a silica pad prior to concentration under reduced pressure. The remainder was purified over silica gel column chromatography (Hex/EA 95:5 to 70:30) to afford the desired aldol product 3 as a single diastereomer in 54% yield.

¹H NMR (400 MHz, CDCl₃) δ 7.39-7.27 (m, 3H), 7.25 (d, J=6.9 Hz, 2H), 5.77 (d, J=6.1 Hz, 1H), 4.79-4.72 (m, 1H), 4.38-4.08 (m, 6H), 3.31 (dd, J=13.6, 3.3 Hz, 1H), 3.27 (bs, 1H), 2.85 (dd, J=13.5, 9.2 Hz, 1H), 1.47 (s, 3H), 1.39 (s, 3H). ¹³C NMR (101 MHz, CDCl₃) δ 168.5, 152.4, 134.7, 129.5, 129.1, 127.6, 109.9, 75.9, 73.7, 66.4, 65.9, 55.5, 52.7, 37.6, 26.6, 25.1.

### (S)-4-benzyl-3-((2S,3R)-3-((tert-butyldimethylsilyl)oxy)-2-chloro-3-((R)-2,2-dimethyl-1,3-dioxolan-4-yl)propanoyl)oxazolidin-2-one (4)

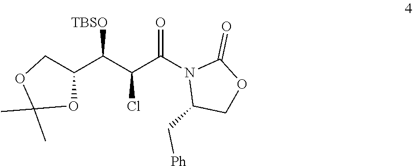 [SMILES: CC1(C)OC[C@H]([C@@H](O[Si](C)(C)C(C)(C)C)[C@H](Cl)C(=O)N2C(=O)OC[C@@H]2Cc2ccccc2)O1]

To a solution of compound 3 (230 mg, 0.60 mmol) in dry CH₂Cl₂ (6 mL) at 0° C. under N₂ was added sequentially 2,6-lutidine (280 2.4 mmol) and TBSOTf (275 μL, 1.2 mmol). The mixture was stirred 1 h at 0° C. and 1 M HCl was added. The mixture was extracted with CH₂Cl₂. Combined organic layers were washed with water, brine, dried over MgSO₄ and concentrated in vacuo. The remainder was purified over silica gel column chromatography (Hex/EA 95:5) to afford the desired sylilated derivative 4 in 94% yield as a white solid. ¹H NMR (400 MHz, CDCl₃) δ 7.47-7.23 (m, 5H), 5.71 (d, J=4.0 Hz, 1H), 4.78-4.70 (m, 1H), 4.54 (dd, J=6.1, 4.0 Hz, 1H), 4.43 (dd, J=13.4, 6.3 Hz, 1H), 4.32-4.22 (m, 1H), 4.10 (dd, J=8.2, 6.4 Hz, 1H), 3.92 (t, J=7.8 Hz, 1H), 3.52 (dd, J=13.3, 2.8 Hz, 1H), 2.65 (dd, J=13.2, 10.6 Hz, 1H), 1.40 (s, 3H), 1.32 (s, 3H), 0.93 (s, 9H). ¹³C NMR (101 MHz, CDCl₃) δ 165.85, 152.91, 135.18, 129.29, 129.14, 127.52, 109.30, 74.63, 72.36, 67.05, 66.31, 62.02, 56.09, 37.97, 26.62, 25.74, 25.45, 18.09, −4.53, −4.89.

### Alternate Workup to Directly Provide the Ethyl Ester:

### Ethyl (3R)-2-chloro-3-((R)-2,2-dimethyl-1,3-dioxolan-4-yl)-3-hydroxypropanoate (38)

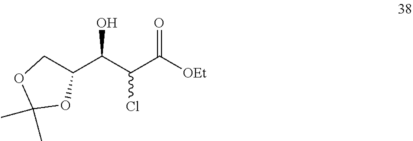 [SMILES: CCOC(=O)C(Cl)[C@H](O)[C@H]1COC(C)(C)O1]

To a solution of LiHMDS (24.8 mL, 24.8 mmol, 1 M in THF) in dry THF (50 mL) at −78° C. under N₂ was added dropwise via cannula a solution of 2 (4.2 g, 16.5 mmol) in THF (15 mL). The mixture was stirred 30 min at this temperature and a solution of freshly distilled (R)-2,2-dimethyl-1,3-dioxolane-4-carbaldehyde (3.91 mL, 24.8 mmol) in THF (15 mL) was added dropwise via cannula. After 30 minutes stirring, the reaction was quenched at −78° C. by dropwise addition of anhydrous ethanol (10 mL). After 10 min stirring, silica was added and the reaction mixture was allowed to warm up to room temperature and filtered over a short pad of silica which was washed with ethyl acetate. The filtrate was concentrated under reduced pressure and the remainder was purified over silica gel column chromatography (hexane/ethyl acetate 9:1 to 7:3) to afford compound 38 (2.17 g, 52%) as a diastereomeric mixture which was used directly in the next step.

### Methyl (2S,3R)-3-((tert-butyldimethylsilyl)oxy)-2-chloro-3-((R)-2,2-dimethyl-1,3-dioxolan-4-yl)propanoate (5)

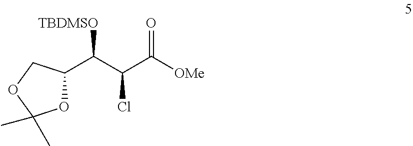 [SMILES: COC(=O)[C@@H](Cl)[C@H](O[Si](C)(C)C(C)(C)C)[C@H]1COC(C)(C)O1]

To a solution of compound 4 (1.25 g, 2.51 mmol) in anhydrous MeOH (27 mL) at −30° C. was added NaOMe (54 mg, 1.0 mmol). The mixture was stirred 30 min at −30° C. and quenched by addition of 1M HCl. After dilution with EtOAc, the mixture was washed with a saturated aqueous solution of NaHCO₃ and brine. The organic layer was dried over MgSO₄ and concentrated in vacuo. The remainder was purified over silica gel column chromatography (Hex/EA (hexane/ethyl acetate) 95:5) to afford the desired methyl ester 5 (691 mg) in 78% yield.

¹H NMR (400 MHz, CDCl₃) δ 4.46 (d, J=2.7 Hz, 1H), 4.18-4.10 (m, 1H), 4.05 (dd, J=7.3, 2.7 Hz, 1H), 3.92 (dd, J=8.5, 6.3 Hz, 1H), 3.75 (dd, J=8.5, 5.1 Hz, 1H), 3.64 (s, 3H), 1.25 (s, 3H), 1.19 (s, 3H), 0.75 (s, 9H), 0.03 (s, 3H), −0.00 (s, 3H). ¹³C NMR (101 MHz, CDCl₃) δ 167.3, 109.5, 75.6, 75.4, 66.5, 61.2, 52.9, 26.5, 25.6, 25.0, 17.9, −4.4, −4.6.

Methyl (2S,3R)-3-((tert-butyldimethylsilyl)oxy)-2-chloro-3-((R)-2,2-dimethyl-1,3-dioxolan-4-yl)-2-fluoropropanoate (6)

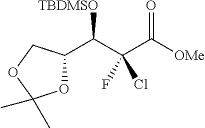 [SMILES: COC(=O)[C@@](F)(Cl)[C@H](O[Si](C)(C)C(C)(C)C)[C@H]1COC(C)(C)O1]

To a solution of compound 5 (673 mg, 1.90 mmol) and NFSI (903 mg, 2.85 mmol) in anhydrous THF (7 mL) at −78° C. under N₂ was added dropwise LiHMDS (1M in THF, 2.5 mL, 2.50 mmol). After 1 h stirring, the reaction was quenched with a saturated aqueous solution of NH₄Cl and extracted with ethyl acetate. Solid potassium permanganate (500 mg) and water (1 mL) were added to the organic layer to remove traces of elimination compound. The mixture was agitated for 2 min and diluted with ethyl acetate (25 mL), a saturated aqueous solution of Na₂S₂O₄ was added until a brown precipitate was observed followed by the addition of HCl 1N until the layers turned colorless with a white precipitate. The organic layer was washed with brine, dried over Na₂SO₄and concentrated under reduced pressure. The crude was purified by flash column chromatography using hexane/ethyl acetate 9:1 as eluent to give compound 6 (592 mg) as a single diastereomer in 84% yield.

¹H NMR (400 MHz, CDCl₃) δ 4.36-4.28 (m, 2H), 4.06 (dd, J=8.8, 6.2 Hz, 1H), 3.92 (dd, J=8.6, 6.0 Hz, 1H), 3.87 (s, 3H), 1.40 (s, 3H), 1.32 (s, 3H), 0.92 (s, 9H), 0.20 (s, 3H), 0.17 (s, 3H). ¹⁹F NMR (377 MHz, CDCl₃) δ −128.36 (d, J=19.4 Hz). ¹³C NMR (101 MHz, CDCl₃) δ 165.3 (d, J=27.9 Hz), 109.1, 104.8 (d, J=261.3 Hz), 77.2 (d, J=21.2 Hz), 75.2 (d, J=3.8 Hz), 65.9, 53.5, 25.9, 25.8, 24.6, 18.3, −4.0, −4.1.

### Example 2

### Method 2—2-Cl Up, 2-F Down

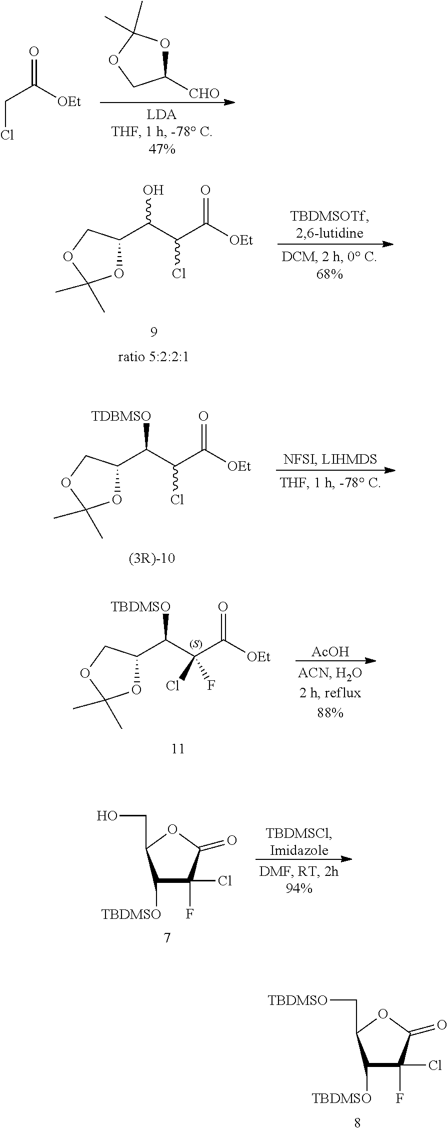

### Ethyl 2-chloro-3-((R)-2,2-dimethyl-1,3-dioxolan-4-yl)-3-hydroxypropanoate (9)

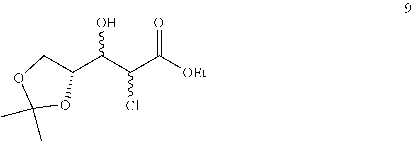 [SMILES: CCOC(=O)C(Cl)C(O)[C@H]1COC(C)(C)O1]

Ethyl chloroacetate (5 g, 40.8 mmol) was added to a solution of LDA (1 M in THF/hexane, 53 mL, 53.0 mmol) in dry THF (300 mL) at −78° C. under N₂. After 15 min stirring, freshly distilled (R)-2,2-dimethyl-1,3-dioxolane-4-carbaldehyde (6.1 mL, 49.0 mmol) was introduced to the mixture and the reaction was stirred for an additional hour at −78° C. The reaction was quenched with HCl 1M at −78° C. The mixture was extracted with ethyl acetate, washed with water, brine, dried over MgSO₄ and concentrated in vacuo. The crude product was purified by flash column chromatography (hexane/ethyl acetate 9:1 to 8:2) to afford compound 9 (4.8 g, 47%) as a diastereomeric mixture which was used directly in the next step.

### Ethyl (3R)-3-((tert-butyldimethylsilyl)oxy)-2-chloro-3-((R)-2,2-dimethyl-1,3-dioxolan-4-yl)propanoate (10)

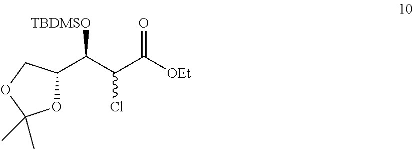 [SMILES: CCOC(=O)C(Cl)[C@H](O[Si](C)(C)C(C)(C)C)[C@H]1COC(C)(C)O1]

To a solution of compound 9 (1.5 g, 5.9 mmol) in dichloromethane (15 mL) at 0° C. was added 2,6-lutidine (2.7 mL, 23.7 mmol) followed by TBDMSOTf (2.7 mL, 11.9 mmol). The reaction mixture was stirred for 2 h then quenched with saturated aqueous solution of NaHCO₃. The crude mixture was extracted with ethyl acetate, washed with HCl 1M then brine, dried over Na₂SO₄, filtered and concentrated under reduced pressure. Purification by flash column chromatography (hexane/ethyl acetate 9/1) afforded the two major diastereomers (2S,3R)-10 and (2R,3R)-10 with a 50% yield (1.1 g, ratio 7:3) and the two minor diastereoisomers (2S,3S)-10 and (2R,3S)-10 with an 18% yield (388 mg, ratio 8:2).

Compounds (2S,3R)-10 and (2R,3R)-10: ¹H NMR (400 MHz, CDCl₃) δ 4.66 (d, J=2.5 Hz, 1H), 4.60 (d, J=2.7 Hz, 0.5H), 4.34-4.13 (m, 6H), 4.08 (dd, J=8.4, 6.1 Hz, 1.5H), 4.10-4.06 (m, 1.5H), 1.44-1.28 (m, 13.5H), 0.90-0.88 (m, 13.5H), 0.19 (s, 1.5H), 0.15 (s, 1.5H), 0.12 (s, 3H), 0.05 (s, 3H).

Compounds (2S,3S)-10 and (2R,3S)-10: ¹H NMR (400 MHz, CDCl₃) δ 4.37-4.16 (m, 5.5H), 4.11-4.02 (m, 1.3H), 3.82 (t, J=7.8 Hz, 1.3H), 1.43-1.29 (m, 11.5H), 0.93 (d, J=1.0 Hz, 2.5H), 0.89 (s, 9H), 0.14 (s, 3H), 0.11 (s, 3.8H).

### Ethyl (2S,3R)-3-((tert-butyldimethylsilyl)oxy)-2-chloro-3-((R)-2,2-dimethyl-1,3-dioxolan-4-yl)-2-fluoropropanoate (11)

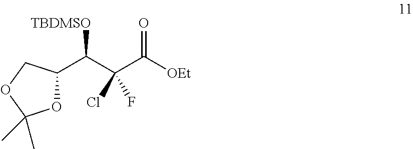 [SMILES: CCOC(=O)[C@@](F)(Cl)[C@H](O[Si](C)(C)C(C)(C)C)[C@H]1COC(C)(C)O1]

A solution of LiHMDS 1 M in THF (32 mL, 32 mmol) was added dropwise to a mixture of compound 10 (8.7 g, 23.7 mmol) and NFSI (11 g, 35.6 mmol) in anhydrous THF (85 mL) at −78° C. After 1 h stirring, the reaction was quenched with a saturated aqueous solution of NH₄Cl and extracted with ethyl acetate. Solid potassium permanganate (3 g) and water (5 mL) were added to the organic layer to remove traces of elimination compound. The mixture was agitated for 2 min. A saturated aqueous solution of Na₂S₂O₃ (100 mL) was added to the heterogeneous mixture. After vigorous shaking, HCl 1M (50 mL) was introduced and the mixture shaked again. This last step can be repeated until disappearance of the brown color. The organic layer was then washed with brine, dried over Na₂SO₄, filtered and concentrated under reduced pressure. The crude was purified by flash column chromatography using hexane/ethyl acetate 9/1 as eluent to give compound 11 as a mixture with unreacted starting material 10 (8.57 g, ratio 7:3, 94%).

¹H NMR (400 MHz, CDCl₃) δ 4.42-4.21 (m, 4H), 4.10-4.00 (m, 1H), 3.93 (dd, J=8.2, 6.4 Hz, 1H), 1.41 (s, 3H), 1.37 (t, J=7.1 Hz, 3H), 1.33 (s, 3H), 0.93 (s, 9H), 0.20 (s, 3H), 0.17 (s, 3H). ¹⁹F NMR (377 MHz, CDCl₃) δ −127.12 (d, J=18.2 Hz). ¹³C NMR (101 MHz, CDCl₃) δ 164.8 (d, J=27.6 Hz), 108.9, 104.8 (d, J=261.4 Hz), 77.2 (d, J=21.4 Hz), 75.3 (d, J=4.0 Hz), 65.6 (d, J=1.5 Hz), 63.1, 25.9, 25.8, 24.6, 18.3, 13.9, −4.0, −4.2, −4.2.

### (2S)-2-Deoxy-2-chloro-2-fluoro-3-O-(tert-butyldimethylsilyl)-5-hydroxy-D-ribonolactone (7)

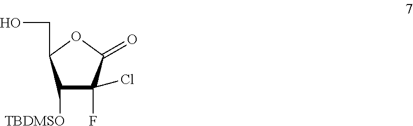

Water (1.7 mL) and acetic acid (8 mL) were introduced to a solution of compound 11/(2R,3R)-10 (8.57 g, 22.3 mmol) in acetonitrile (17 mL). The reaction mixture was refluxed for 2 h then toluene was added. After removal of the solvents under reduced pressure, the crude was purified by flash column chromatography (hexane/ethyl acetate 7/3) to afford pure chloro-fluoro-lactone 7 (3.89 g, 58%).

¹H NMR (400 MHz, CDCl₃) δ 4.56 (dd, J=13.2, 6.5 Hz, 1H), 4.41 (dt, J=6.4, 3.1 Hz, 1H), 4.09 (dd, J=13.0, 2.3 Hz, 1H), 3.83 (d, J=12.9 Hz, 1H), 2.02 (brs, 1H), 0.95 (s, 9H), 0.25 (s, 3H), 0.20 (s, 3H). ¹⁹F NMR (377 MHz, CDCl₃) δ −134.91 (d, J=13.3 Hz). ¹³C NMR (101 MHz, CDCl₃) δ 164.9 (d, J=25.7 Hz), 102.4 (d, J=258.3 Hz), 83.4, 74.2 (d, J=16.3 Hz), 59.2, 25.5, 18.0, −4.3, −5.2.

### (2S)-2-Deoxy-2-chloro-2-fluoro-3,5-di-O-(tert-butyldimethylsilyl)-D-ribonolactone (8)

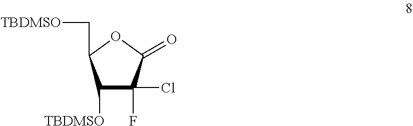

Imidazole (2.3 g, 52 mmol) was added to a mixture of compound 7 (3.89 g, 13 mmol) and TBDMSCl (3.9 g, 26 mmol) in DMF (30 mL). The mixture was stirred 2 h at 25° C., quenched with water and the crude was extracted with ethyl acetate/diethyl ether (1:1). The organic layer was washed with water (3×30 mL) then saturated aqueous solution of NH₄Cl (30 mL) then brine (30 mL), dried over Na₂SO₄, filtrated and concentrated under reduced pressure. The crude was purified by flash column chromatography using hexane/ethyl acetate (95:5) to afford protected lactone 8 (5 g, 94%).

¹H NMR (400 MHz, CDCl₃) δ 4.49 (dd, J=11.9, 5.8 Hz, 1H), 4.29-4.21 (m, 1H), 3.89 (dd, J=12.1, 3.9 Hz, 1H), 3.75 (dd, J=12.1, 3.1 Hz, 1H), 0.84 (s, 9H), 0.80 (s, 9H), 0.12 (s, 3H), 0.08 (s, 3H), −0.00 (s, 3H), −0.01 (s, 3H). ¹⁹F NMR (377 MHz, CDCl₃) δ −134.67 (d, J=11.9 Hz). ¹³C NMR (101 MHz, CDCl₃) δ 165.2 (d, J=26.0 Hz), 102.1 (d, J=259.6 Hz), 83.6, 74.1 (d, J=16.0 Hz), 59.5, 25.7, 25.5, 18.2, 18.0, −4.4, −5.2, −5.4, −5.5.

### Example 3

### Method 1 2-Br Up, 2-F Down

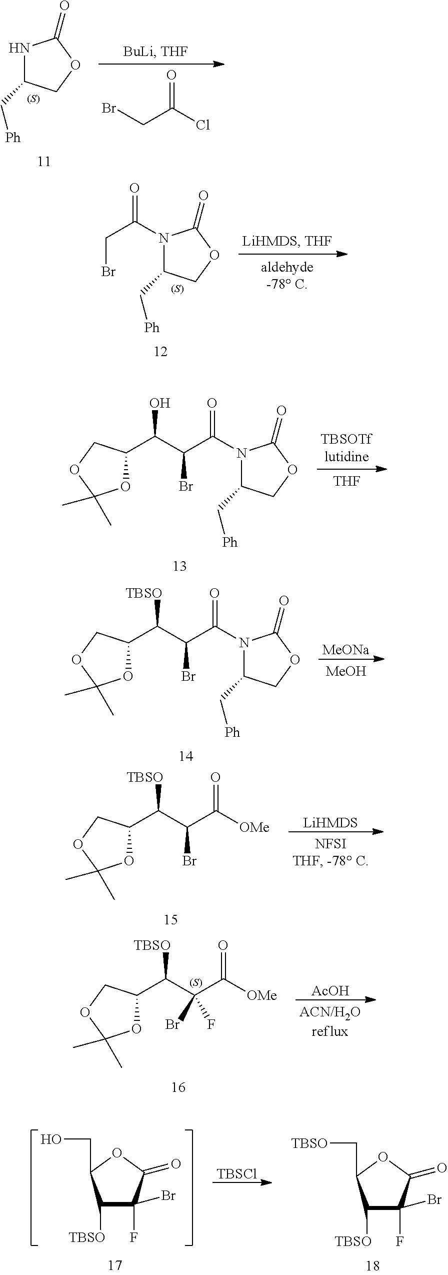

### Example 4

### Method 2 2-Br Up, 2-F Down

### Synthesis of 2-Deoxy-2-fluoro-2-bromo-3,5-di-O-(tert-butyldimethylsilyl)-D-ribono-lactone

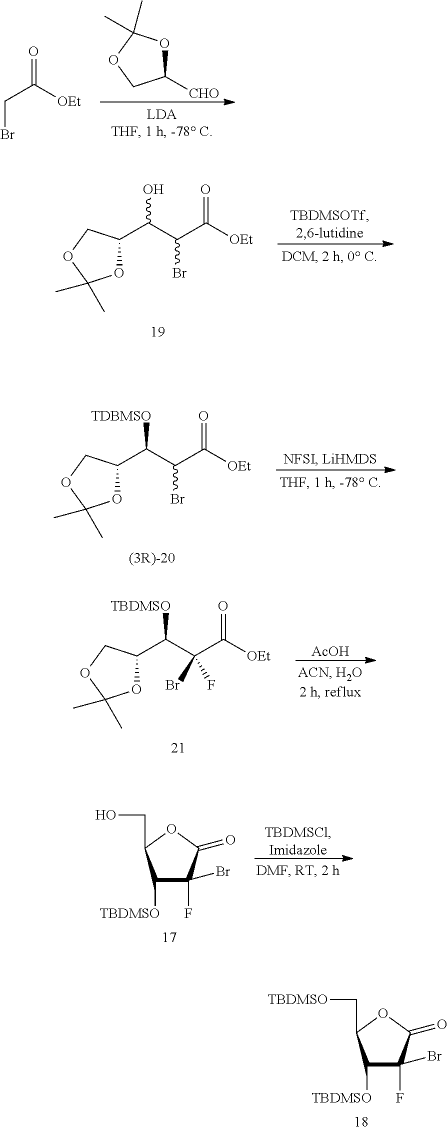

### Ethyl 2-bromo-3-((R)-2,2-dimethyl-1,3-dioxolan-4-yl)-3-hydroxypropanoate (19)

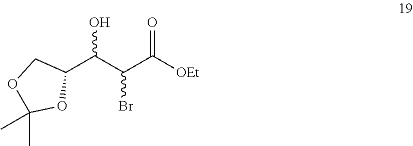 [SMILES: CCOC(=O)C(Br)C(O)[C@H]1COC(C)(C)O1]

A mixture of ethyl bromoacetate (500 mg, 2.99 mmol) and freshly distilled (R)-2,2-dimethyl-1,3-dioxolane-4-carbaldehyde (584 mg, 4.5 mmol) in anhydrous THF (2 mL) was added to a solution of LiHMDS (1 M in THF/hexane, 5 mL, 5 mmol) in dry THF (15 mL) at −78° C. under N₂. The reaction was stirred at −78° C. for 45 min then the mixture was allowed to warm up slowly to -25° C. over 2 h. The reaction was quenched with s saturated aqueous solution of NH₄Cl at −78° C. The mixture was extracted with ethyl acetate, washed with HCl 1M, brine, dried over Na₂SO₄ and concentrated in vacuo. The crude product was purified by flash column chromatography (hexane/ethyl acetate 9:1 to 7:3) to afford compound 19 (280 mg, 31%) as a diastereomeric mixture which was used directly in the next step.

### Ethyl (3R)-3-((tert-butyldimethylsilyl)oxy)-2-bromo-3-((R)-2,2-dimethyl-1,3-dioxolan-4-yl)propanoate (20)

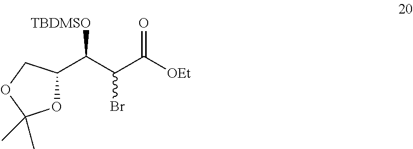 [SMILES: CCOC(=O)C(Br)[C@H](O[Si](C)(C)C(C)(C)C)[C@H]1COC(C)(C)O1]

To a solution of compound 19 (647 mg, 2.17 mmol) in dichloromethane (6 mL) at 0° C. was added 2,6-lutidine (1 mL, 4.3 mmol) followed by TBDMSOTf (1 mL, 8.7 mmol). The reaction mixture was stirred for 2 h then quenched with saturated aqueous solution of NaHCO₃. The crude mixture was extracted with ethyl acetate, washed with HCl 1M then brine, dried over Na₂SO₄, filtered and concentrated under reduced pressure. Purification by flash column chromatography (hexane/ethyl acetate 9/1) afforded major diastereomers (2S,3R)-20 with (2R,3R)-20 in a 59% yield (526 mg, ratio 8:2).

¹H NMR (400 MHz, CDCl3) δ 4.66 (d, J=2.7 Hz, 0.8H), 4.50 (d, J=3.8 Hz, 0.2H), 4.28-4.11 (m, 4H), 4.10-4.02 (m, 1H), 3.93-3.87 (m, 1H), 1.42-1.40 (m, 3H), 1.37-1.28 (m, 6H), 0.90-0.88 (m, 9H), 0.17 (s, 0.6H), 0.14 (s, 0.6H), 0.12 (s, 2.4H), 0.06 (s, 2.4H).

### Ethyl (2S,3R)-3-((tert-butyldimethylsilyl)oxy)-2-bromo-3-((R)-2,2-dimethyl-1,3-dioxolan-4-yl)-2-fluoropropanoate (21)

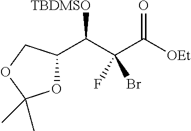 [SMILES: CCOC(=O)[C@@](F)(Br)[C@H](O[Si](C)(C)C(C)(C)C)[C@H]1COC(C)(C)O1]

A solution of LiHMDS 1 M in THF (1.7 mL, 1.7 mmol) was added dropwise to a mixture of compound 12 (525 mg, 1.28 mmol) and NFSI (604 mg, 1.92 mmol) in anhydrous THF (5 mL) at −78° C. After 1 h stirring, the reaction was quenched with a saturated aqueous solution of NH₄Cl and extracted with ethyl acetate. Solid potassium permanganate (300 mg) and water (1 mL) were added to the organic layer to remove traces of elimination compound. The mixture was agitated for 2 min then more water (10 mL) and ethyl acetate (10 mL) were introduced. A saturated aqueous solution of Na₂S₂O₃ (20 mL) was added to the heterogeneous mixture. After vigorous shaking, HCl 1M (10 mL) was introduced and the mixture shaked again. This last step can be repeated until disappearance of the brown color. The organic layer was then washed with brine, dried over Na₂SO₄, filtered and concentrated under reduced pressure. The crude was purified by filtration on a silica pad using hexane/ethyl acetate 9/1 as eluent to give compound 21 (421 mg, 77%).

¹H NMR (400 MHz, CDCl₃) δ 4.48-4.25 (m, 4H), 4.05-4.01 (m, 1H), 3.93 (dd, J=8.2, 6.6 Hz, 1H), 1.41 (s, 3H), 1.36 (t, J=7.1 Hz, 3H), 1.33 (s, 3H), 0.93 (s, 9H), 0.22 (s, 3H), 0.17 (s, 3H). ¹⁹F NMR (377 MHz, CDCl₃) δ −126.19 (d, J=17.9 Hz). ¹³C NMR (101 MHz, CDCl₃) δ 165.3 (d, J=26.2 Hz), 108.8, 98.8 (d, J=271.1 Hz), 77.8 (d, J=20.2 Hz), 75.6 (d, J=4.0 Hz), 65.5, 63.0, 25.9, 25.9, 24.5, 18.4, 13.8, −3.9, −4.1 (d, J=2.1 Hz).

### (2S)-2-Deoxy-2-bromo-2-fluoro-3-O-(tert-butyldimethylsilyl)-5-hydroxy-D-ribonolactone (17)

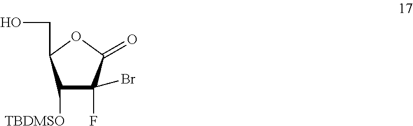

Water (0.07 mL) and acetic acid (0.33 mL) were introduced to a solution of compound 13/(2R,3R)-12(234 mg, 0.54 mmol) in acetonitrile (1.2 mL). The reaction mixture was refluxed for 2 h then toluene was added. After removal of the solvents under reduced pressure, the crude was purified by flash column chromatography (hexane/ethyl acetate 7/3) to afford bromo-fluoro-lactone 17 (135 mg, 72%).

¹H NMR (400 MHz, CDCl₃) δ 4.67 (dd, J=9.8, 5.0 Hz, 1H), 4.45 (dd, J=8.3, 3.7 Hz, 1H), 4.03 (dd, J=12.9, 3.9 Hz, 1H), 3.89 (dd, J=12.9, 4.5 Hz, 1H), 2.61 (brs, 1H), 0.93 (s, 9H), 0.23 (s, 3H), 0.19 (s, 3H). ¹⁹F NMR (377 MHz, CDCl₃) δ −136.89 (d, J=9.7 Hz). ¹³C NMR (101 MHz, CDCl₃) δ 166.1 (d, J=25.2 Hz), 94.5 (d, J=272.8 Hz), 85.1, 75.3 (d, J=15.6 Hz), 59.7, 25.5, 18.0, −4.3, −5.2.

### (2S)-2-Deoxy-2-chloro-2-fluoro-3,5-di-O-(tert-butyldimethylsilyl)-D-ribonolactone (18)

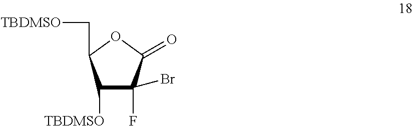

Imidazole (68 mg, 0.98 mmol) was added to a mixture of compound 17 (135 mg, 0.39 mmol) and TBDMSCl (118 mg, 0.77 mmol) in DMF (1 mL). The mixture was stirred 3 h at 25° C., quenched with water and the crude was extracted with ethyl acetate/diethyl ether (1:1). The organic layer was washed with water (3×10 mL) then saturated aqueous solution of NH₄Cl then brine, dried over Na₂SO₄, filtrated and concentrated under reduced pressure. The crude was purified by flash column chromatography using hexane/ethyl acetate (95:5) to afford protected lactone 18 (179 mg, quantitative yield).

¹H NMR (400 MHz, CDCl₃) δ 4.74 (dd, J=8.6, 4.3 Hz, 1H), 4.40 (dd, J=7.9, 3.8 Hz, 1H), 4.00 (dd, J=11.7, 5.3 Hz, 1H), 3.91 (dd, J=11.7, 3.9 Hz, 1H), 0.94 (s, 9H), 0.91 (s, 9H), 0.23 (s, 3H), 0.19 (s, 3H), 0.11 (s, 3H), 0.10 (s, 3H). ¹⁹F NMR (377 MHz, CDCl₃) δ −136.64 (d, J=8.6 Hz). ¹³C NMR (101 MHz, CDCl₃) δ 165.9 (d, J=25.4 Hz), 94.1 (d, J=274.0 Hz), 84.7, 75.1 (d, J=15.4 Hz), 60.1, 25.8, 25.5, 18.3, 18.1, −4.4, −5.2, −5.5.

### Example 5

### Method 3, 2-Cl Up, 2-F Down

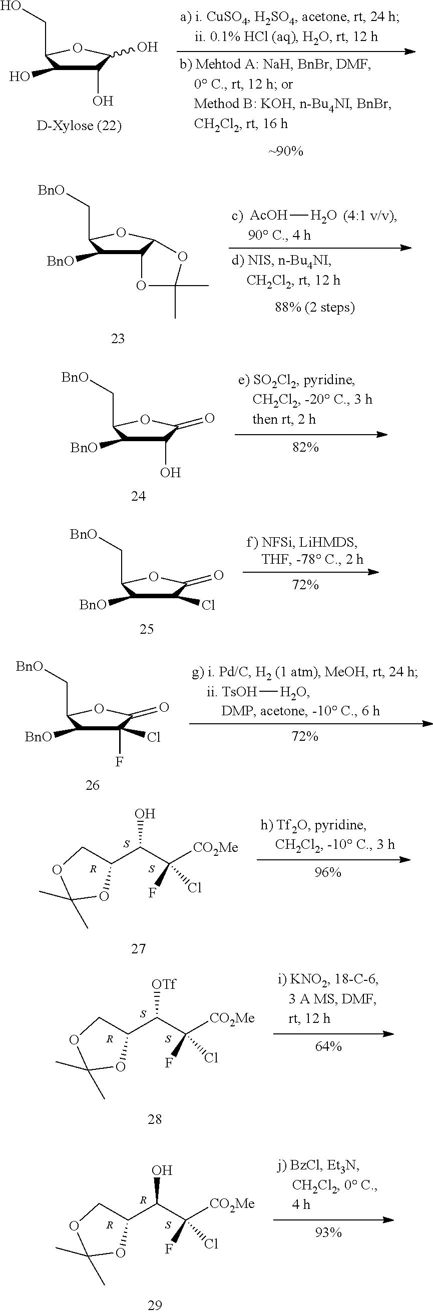

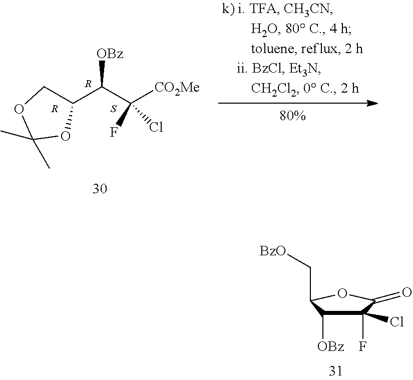

### (3aR,5R,6aR)-6-(Benzyloxy)-5-((benzyloxy)methyl)-2,2-dimethyltetrahydrofuro[2,3-d][1,3]dioxole (3)^{ref 1}

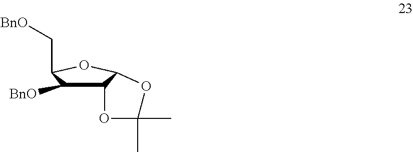 [SMILES: CC1(C)O[C@H]2O[C@H](COCc3ccccc3)[C@H](OCc3ccccc3)[C@@H]2O1]

To a solution of D-xylose (30.0 g, 0.20 mol) in 800 mL of acetone was treated with CuSO₄ (60.0 g, 0.38 mol) and H₂SO₄ (2.0 mL) at 0° C. under N₂ atmosphere. After being stirred for 12 h at room temperature, the solution was neutralized with Na₂CO₃ at 0° C., and filtered out all white solid by using a celite pad. The filtrate was concentrated under reduced pressure to provide 1,2,3,5-diisopropylidenyl xylose. The xylose derivative was dissolved in 300 mL of aqueous 0.1% hydrochloric acid at room temperature and stirred for 12 h. The resulting solution was neutralized with NaHCO₃ at 0° C. to reach pH 7 and the solid was removed by using a celite pad. The filtrate was concentrated under reduced pressure and the residue was purified by silica gel pad (EtOAc) to give a 1,2-isopropylidenyl xylose (31.0 g).

Method A: To a solution of 1,2-isopropylidenyl xylose (14.0 g, 73.57 mmol) in 200 mL of DMF was added NaH (6.62 g, 0.166 mol, 60% dispersion in mineral oil) over 30 min at 0° C. under N₂ atmosphere. After being stirred for 1 h, benzyl bromide (31.25 g, 0.19 mol) was added dropwise to the solution at 0° C. under N₂ atmosphere. The resulting solution was stirred for 12 h at room temperature and treated with 10 mL of saturated NH₄Cl (aq) at 0° C. and additionally stirred for 10 min then poured into a mixture of EtOAc-water (300 mL, 2:1 v/v). The organic layer was separated and aqueous layer was washed with EtOAc (200 mL×2). The combined organic layers were washed with brine (100 mL), dried over Na₂SO₄ and filtered. The filtrate was concentrated under reduced pressure and purified by silica gel (hexanes:EtOAc=10:1 v/v) to give compound 23 (26.44 g, 71.36 mmol) in 97% yield.

Method B: To a mixture of 1,2-isopropylidenyl xylose (117.50 g, 0.62 mol), 85% potassium hydroxide (122.34 g, 1.85 mol), and tetra-butylammonium iodide (68.50 g, 0.19 mol) in DCM 1.20 L was added benzyl bromide (264.20 g, 1.55 mol) at room temperature, After being stirred for 16 h. The reaction mixture was diluted with n-hexanes (1.20 L), washed with brine (600 ml×2), dried over Na₂SO₄, filtered and evaporated under reduced pressure. The residue was purified with silica gel column chromatography (hexanes:EtOAc=10:1 v/v) to give compound 23 (200.90 g, 0.54 mol) in 88% yield. ¹H NMR (400 MHz, CDCl₃) δ 7.37-7.28 (m, 10H), 5.98 (d, J=3.8 Hz, 1H), 4.71-4.63 (m, 3H), 4.56 (d, J=4.5 Hz, 1H), 4.53 (d, J=4.6 Hz, 1H), 4.44 (m, 1H), 4.01 (d, J=3.2, 1H), 3.84-3.78 (m, 2H), 1.52 (s, 3H), 1.5 (s, 3H); ¹³C NMR (100 MHz, CDCl₃) δ 138.1, 137.6, 128.5, 128.4, 127.9, 127.8, 127.7, 127.6, 111.7, 105.1, 82.4, 81.7, 79.3, 73.6, 72.0, 67.6, 26.8, 26.3.

Reference

1. Bioorg. Med. Chem. 2006, 14, 500-509

### (3R,5R)-4-(Benzyloxy)-5-((benzyloxy)methyl)-3-hydroxydihydrofuran-2(3H)-one (24)

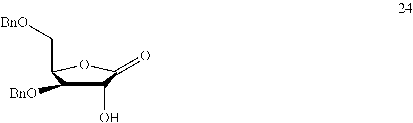

Compound 23 (25.0 g, 67.49 mmol) was dissolved in 160 mL of acetic acid and 40 ml of water at room temperature and then heated to 90° C. After being stirred for 4 h, the solution was cooled to room temperature and concentrated under reduced pressure. The residue was dissolved in 100 mL of toluene and concentrated in vacuo, repeated two more times. To solution of the residue in 100 mL of MeOH was treated with K₂CO₃ (1.0 g) at 0° C. under N₂ atmosphere. After being stirred for 1 h at room temperature, the solution was neutralized with methanolic hydrogen chloride solution at 0° C. and adsorbed on silica gel and then purified by silica gel column (hexanes:EtOAc=10:1 to 2:1 v/v) to give 3,5-dibenzyl xylose (21.5 g, 65.08 mmol) in 96% yield. To a solution of 3,5-dibenzyl xylose (16.50 g, 49.94 mmol) in 150 mL of CH₂Cl₂ was added a solution of N-iodosuccinimide (29.21 g, 129.85 mmol) and n-Bu₄NI (59.93 g, 22.14 mmol) in 200 mL of CH₂Cl₂ at 0° C. under N₂ atmosphere. After being stirred for 20 h at room temperature, the solution was poured into cold saturated Na₂S₂O₃ (aq) (150 mL) and organic layer was separated. The aqueous layer was washed with CH₂Cl₂ (100 mL×2) and the combined organic layer was washed with brine (100 mL), dried over Na₂SO₄ and filtered. The filtrate was concentrated under reduced pressure and the residue was purified by silica gel pad (hexanes:EtOAc=5:1 to 1:1 v/v) to give a 3,5-dibenzyl xylolactone (24) (15.91 g, 48.44 mmol) in 97% yield. ¹H NMR (400 MHz, CDCl₃) δ 7.38-7.29 (m, 10H), 4.88-4.85 (m, 1H), 4.69 (d, J=11.8 Hz, 1H), 4.63-4.53 (m, 3H), 4.40 (t, J=8.0 Hz, 1H), 3.78 (ddd, J=2.4, 10.8, 30.8 Hz, 2H), 2.88 (d, J=2.8, 1H); ¹³C NMR (100 MHz, CDCl₃) δ 175.3, 137.6, 137.3, 128.6, 128.5, 128.1, 127.8, 127.7, 127.5, 80.4, 77.1, 73.6, 72.7, 72.3, 66.9.

### (5R)-4-(Benzyloxy)-5-((benzyloxy)methyl)-3-chlorodihydrofuran-2(3H)-one (25)

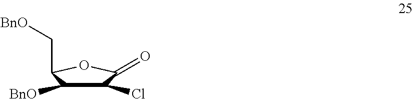

Method A: To a solution of compound 24 (15.62 g, 47.56 mmol) and pyridine (9.03 g, 114.15 mmol) in 200 mL of anhydrous CH₂Cl₂ was added a solution of sulfuryl chloride (9.63 g, 71.36 mmol) in 70 mL of CH₂Cl₂ at −10° C. under N₂ atmosphere. After being stirred for 2 h, the reaction mixture was treated with pyridine (4.50 g, 57.08 mmol) and warmed to room temperature and additionally stirred for 4 h under N₂ atmosphere. The solution was diluted with diethyl ether (500 mL) and washed with saturated NaHCO₃ (aq) (200 mL), brine (100 mL), dried over Na₂SO₄ and filtered. The filtrate was concentrated under reduced pressure and the residue was purified by silica gel column chromatography (hexanes:EtOAc=3:1 v/v) to give compound 25 (13.0 g, 37.49 mmol) in 79% yield.

Method B: To a solution of compound 24 (78.20 g, 0.24 mol), pyridine (115.60 mL, 1.43 mol) in 400 mL of CH₂Cl₂ at −10° C. under N₂ atmosphere was added a solution of sulfuryl chloride (38.60 mL, 0.48 mol) in 400 ml of CH₂Cl₂ over 1 h, dropwise. After being stirred for 1 h at the same temperature, the reaction mixture was warmed to 0° C. over 1 h, and then stirred for 1 h, additionally. After being stirred for 21 hour at room temperature, the reaction mixture was treated with saturated NH₄Cl aqueous solution (400 mL), and the organic layer was separated. The organic layer was washed with saturated NaHCO₃ aqueous solution (400 mL×2), dried over Na₂SO₄, filtered and evaporated under reduced pressure. The residue was purified by silica gel column chromatography (hexanes:EtOAc=5:1 to 3:1 v/v to give compound 25 (63.20 g, 0.18 mol) in 76% yield. ¹H NMR (400 MHz, CDCl₃) δ 7.43-7.30 (m, 10H), 4.86 (d, J=12.0 Hz, 1H), 4.73 (d, J=6.4 Hz, 1H), 4.72-4.69 (m, 1H), 4.68 (d, J=12.0 Hz, 1H), 4.63-4.56 (m, 2H), 4.44 (t, J=6.4 Hz, 1H), 3.82 (ddd, J=3.2, 12.0, 40.0 Hz, 2H); ¹³C NMR (100 MHz, CDCl₃) δ 170.4, 137.4, 136.6, 128.7, 128.5, 128.4, 127.9, 127.8, 127.7, 81.5, 78.8, 73.8, 73.0, 66.9, 54.4.

### (3S,5R)-4-(benzyloxy)-5-((benzyloxy)methyl)-3-chloro-3-fluorodihydrofuran-2(3H)-one (5)

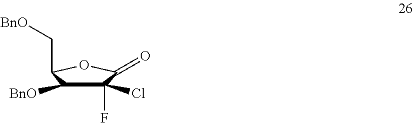

To a solution of compound 25 (12.0 g, 34.60 mmol) and NFSI (13.64 g, 43.25 mmol) in 200 mL of anhydrous THF was added LiHMDS (34.60 mL, 34.60 mmol, 1.0 M in THF) dropwise at −78° C. under Ar atmosphere. After being stirred for 1 h at the same temperature, the reaction mixture was treated with 34 mL of saturated NH₄Cl (aq) and stirred for 10 min. The resulting slurry was poured into saturated cold NH₄Cl (aq) (200 mL) and EtOAc (400 mL). The organic layer was separated and the aqueous layer was washed with EtOAc (200 mL×2). The collected organic layers were washed with brine (200 mL), dried over Na₂SO₄ and filtered. The filtrate was concentrated under reduced pressure and the residue was purified by silica gel column chromatography (hexanes:EtOAc=10:1 to 3:1 v/v) to give compound 26 (9.09 g, 24.91 mmol) in 72% yield. ¹H NMR (400 MHz, CDCl₃) δ 7.44-7.31 (m, 10H), 4.97 (d, J=12.0 Hz, 1H), 4.87-4.83 (m, 1H), 4.65 (d, J=12.0 Hz, 1H), 4.63-4.53 (m, 3H), 3.89 (dd, J=4.0, 11.2 Hz, 1H), 3.89 (dd, J=7.2, 11.2 Hz, 1H); ¹³C NMR (100 MHz, CDCl₃) δ 165.0, 137.3, 135.7, 128.7, 128.6, 128.5, 128.3, 128.0, 127.8, 78.4, 78.0, 77.8, 73.7, 73.6, 67.1.

### Methyl (2R,3S)-2-chloro-3-((R)-2,2-dimethyl-1,3-dioxolan-4-yl)-2-fluoro-3-hydroxypropanoate (27)

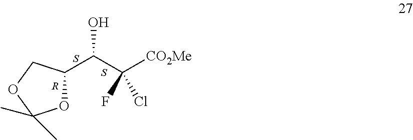

A solution of compound 26 (1.0 g, 2.74 mmol) in 10.0 mL of MeOH was added Pd/C (0.11 g, 10% Pd on activated carbon) at room temperature and stirred for 12 h under H₂ atmosphere (1 atm, balloon). Celite (0.50 g) was added and the resulting slurry was stirred for 30 min, the suspension was filtered and washed with MeOH (10 mL×5). The collected solution was concentrated under reduced pressure and the residue was dried under high vacuum for 12 h at room temperature. To a solution of the product in 10.0 mL of acetone was added TsOH—H₂O (26.1 mg, 0.14 mmol) and 2,2-dimethoxypropane (1.71 g, 16.44 mmol) at −10° C. under N₂ atmosphere. After being stirred for 6 h, the reaction mixture was neutralized with K₂CO₃ (40.0 mg, 0.28 mmol) at −10° C., and stirred for 30 min, filtered off a solid. The filtrate was concentrated under reduced pressure and the residue was purified by silica gel column (hexanes:EtOAc=10:1 to 4:1 v/v) to give compound 27 (0.51 g, 1.97 mmol) in 72% yield. ¹H NMR (400 MHz, CDCl₃) δ 4.64 (dt, J=1.2, 6.4 Hz, 1H), 4.21 (dd, J=6.8, 8.2 Hz, 1H), 4.05 (ddd, J=1.9, 10.0, 20.0 Hz, 1H), 4.01-3.89 (m, 1H), 3.92 (s, 3H), 3.24 (d, J=10.0 Hz, 1H), 1.46 (s, 3H), 1.43 (s, 3H); ¹³C NMR (100 MHz, CDCl₃) δ 165.2 (d, J=29.0 Hz), 110.6, 103.6 (d, J=261.0 Hz), 73.7 (d, J=22.7 Hz), 71.7, 67.1, 53.9, 26.0, 25.3; ¹⁹F NMR (377 MHz, CDCl₃) δ −135.60 (d, J=19.9 Hz).

### Methyl (2R,3S)-2-chloro-3-((R)-2,2-dimethyl-1,3-dioxolan-4-yl)-2-fluoro-3-(((trifluoromethyl)sulfonyl)oxy)propanoate (28)

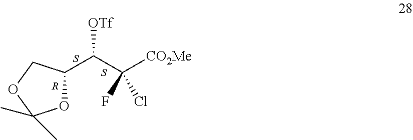

To a solution of compound 27 (1.90 g, 7.40 mmol) in 40 mL of CH₂Cl₂ was added anhydrous pyridine (2.34 g, 29.60 mmol) and trifluoromethanesulfonic anhydride (4.18 g, 14.80 mmol) at −20° C. under argon atmosphere. After being stirred for 3 h at the same temperature, the solution was diluted with diethyl ether (100 mL) and washed with cold 0.5N HCl (aq) (30 mL×2) and bine (30 mL), dried over Na₂SO₄ and filtered. The filtrate was concentrated under reduced pressure and the residue was purified by silica gel column (hexanes:EtOAc=10:1 to 3:1 v/v) to give compound 28 (2.76 g, 7.11 mmol) in 96% yield. ¹H NMR (400 MHz, CDCl₃) δ 5.29 (dd, J=7.8, 15.6 Hz, 1H), 4.56 (q, J=6.8 Hz, 1H), 4.31 (dd, J=6.4, 9.2 Hz, 1H), 4.09 (dd, J=6.4, 9.2 Hz, 1H), 3.97 (s, 3H), 1.48 (s, 3H), 1.42 (s, 3H); ¹³C NMR (100 MHz, CDCl₃) δ 162.8 (d, J=28.4 Hz), 118.5 (d, J=236.0 Hz), 110.9, 100.4 (d, J=262.0 Hz), 86.0 (d, J=23.0 Hz), 71.7 (d, J=3.0 Hz), 66.9, 54.6, 25.6, 25.2; ¹⁹F NMR (377 MHz, CDCl₃) δ −74.51, −129.70 (d, J=15.3 Hz).

### (1R,2R)-2-Chloro-1-((R)-2,2-dimethyl-1,3-dioxolan-4-yl)-2-fluoro-3-methoxy-3-oxopropyl benzoate (29)

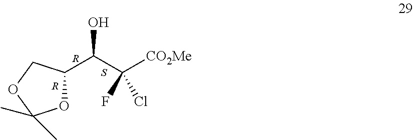

To a solution of KNO₂ (1.18 g, 13.83 mmol), 18-crown-6 (1.83 g, 6.91 mmol) and 3 Å MS (1.20 g) in 20 mL of DMF was added a solution of compound 28 (2.15 g, 5.53 mmol) in 4 mL of THF at room temperature under argon atmosphere. After being stirred for 12 h, the resulting solution was poured into a mixture of EtOAc-H₂O (100 mL, 4:1 v/v) and stirred for 1 h. The organic layer was separated and washed with cold water (20 mL×2) and brine (30 mL), dried over Na₂SO₄ and filtered. The filtrate was concentrated under reduced pressure and the residue was purified by silica gel column (hexanes:EtOAc=5:1 to 2:1 v/v) to give compound 29 (0.91 g, 3.55 mmol) in 64% yield. ¹H NMR (400 MHz, CDCl₃) δ 4.21-4.15 (m, 2H), 4.15-4.07 (m, 2H), 3.89 (s, 3H), 2.82 (d, J=4.8 Hz, 1H), 1.38 (s, 3H), 1.33 (s, 3H); ¹³C NMR (100 MHz, CDCl₃) δ 165.1 (d, J=28.0 Hz), 110.4, 105.6 (d, J=262.0 Hz), 75.8 (d, J=21.0 Hz), 73.9 (d, J=3.0 Hz), 67.2, 53.6, 26.1, 24.9; ¹⁹F NMR (377 MHz, CDCl₃) δ −135.19 (d, J=20.9 Hz).

### (1R,2R)-2-Chloro-1-((R)-2,2-dimethyl-1,3-dioxolan-4-yl)-2-fluoro-3-methoxy-3-oxopropyl benzoate (30)

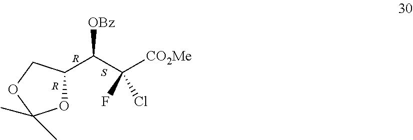

To a solution of compound 29 (0.60 g, 2.34 mmol) in 10 mL of anhydrous CH₂Cl₂ was added benzoyl chloride (0.41 g, 2.92 mmol) and Et₃N (0.36 g, 3.51 mmol) at 0° C. under argon atmosphere. After being stirred for 6 h at the same temperature, the solution was warmed to room temperature over 30 min and stirred for 30 min. The solution was adsorbed on silica gel and purified on silica gel column (hexanes:EtOAc=20:1 to 10:1 v/v) to give compound 30 (0. 79 g, 2.18 mmol) in 93% yield. ¹H NMR (400 MHz, CDCl₃) δ 8.13 (d, J=7.4 Hz, 2H), 7.64 (t, J=7.2 Hz, 1H), 5.34 (t, J=7.6 Hz, 2H), 5.86 (dd, J=8.0, 22.4 Hz 1H), 4.43 (dd, J=6.0, 13.2 Hz, 1H), 4.12 (dd, J=6.4, 8.8 Hz, 1H), 3.99 (dd, J=5.2, 8.8 Hz, 1H), 3.94 (s, 3H), 1.38 (s, 3H), 1.34 (s, 3H); ¹³C NMR (100 MHz, CDCl₃) δ 165.0, 164.3 (d, J=28.0 Hz), 133.9, 130.2, 128.7, 128.5, 110.7, 103.3 (d, J=259.0 Hz), 74.7 (d, J=20.4 Hz), 73.0 (d, J=2.8 Hz), 66.6, 53.8, 26.0, 25.0; ¹⁹F NMR (377 MHz, CDCl₃) δ −131.78 (d, J=22.6 Hz).

### ((2R,3R,4R)-3-(Benzoyloxy)-4-chloro-4-fluoro-5-oxotetrahydrofuran-2-yl)methyl benzoate (31)

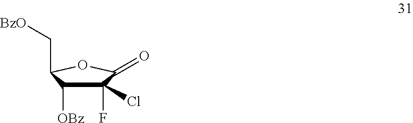

To a solution of compound 30 (0.32 g, 0.89 mmol) in 4.0 mL of CH₃CN was added 0.20 mL of water and 0.03 mL of TFA at room temperature. After being stirred for 10 min at the same temperature, the reaction solution was refluxed at 80° C. for 4 h and cooled to room temperature and then concentrated under reduced pressure. The round-bottomed reaction flask containing the residue, equipped with a Dean-Stark water separator apparatus was charged with 4.0 mL of anhydrous toluene. The clean solution was refluxed at 120° C. (bath temperature) to remove water and cyclize a lactone ring. After being stirred for 2 h at the same temperature, the reaction solution was cooled to room temperature over 30 min and then concentrated under reduced pressure at 40° C. (bath temperature). The residue was dried under high vacuum for 6 h at room temperature. To a solution of the residue in 5 mL of anhydrous CH₂Cl₂ was added BzCl (0.17 g, 1.20 mmol) and Et₃N (0.15 g, 1.43 mmol) at 0° C. under argon atmosphere. After being stirred at 0° C. for 2 h, the resulting solution was diluted with CH₂Cl₂ (10 mL) and washed with cold 0.5N HCl (5 mL) and brine (10 mL), dried over Na₂SO₄ and filtered. The filtrate was concentrated under reduced pressure and the residue was purified by silica gel column (hexanes:EtOAc=20:1 to 10:1 v/v) to give compound 31 (0.28 g, 0.71 mmol) in 80% yield. ¹H NMR (400 MHz, CDCl₃) ¹H NMR (400 MHz, CDCl₃) δ 8.14-8.06 (m, 4H), 7.68 (t, J=7.2 Hz, 1H), 7.62 (t, J=7.6 Hz, 1H), 7.54-7.46 (m, 4H), 5.90 (dd, J=3.6, 4.8 Hz, 1H), 5.06-5.03 (m, 1H), 4.82-4.72 (m, 2H); ¹³C NMR (100 MHz, CDCl₃) δ 165.8, 164.5, 164.0 (d, J=27.6 Hz), 134.5, 133.7, 130.2, 129.9, 128.8, 128.7, 128.6, 127.5, 98.9 (d, J=265.8 Hz), 79.3, 73.7 (d, J=15.1 Hz), 62.2; ¹⁹F NMR (377 MHz, CDCl₃) δ −135.39 (d, J=4.5 Hz).

### Example 6

### Method 3, Synthesis of 2-Br,2-F-Ribolactone Analogs

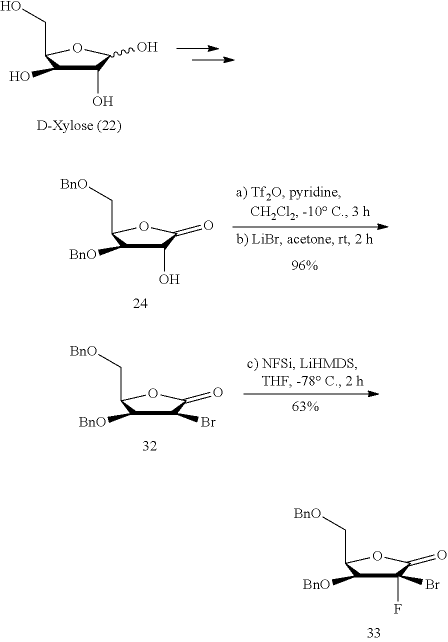

### (3R,5R)-4-(Benzyloxy)-5-((benzyloxy)methyl)-3-bromodihydrofuran-2(3H)-one (32)

To a solution of compound 24 (1.0 g, 3.05 mmol) and pyridine (0.73 g, 9.15 mmol) in 12 mL of anhydrous CH₂Cl₂ was added trifluoromethanesulfonic anhydride (1.28 g, 4.56 mmol) at −10° C. under N₂ atmosphere. After being stirred for 2 h, the reaction mixture was diluted with diethyl ether (40 mL) and washed with cold 0.5 N-HCl (aq) (20 mL×2), brine (20 mL), dried over Na₂SO₄ and filtered. The filtrate was concentrated under reduced pressure and purified by silica gel column chromatography (hexanes:EtOAc=5:1 to 2:1 v/v) to give compound. To a solution of the triflate (1.41 g, 3.05 mmol) in acetone (25 mL) was added lithium bromide (1.32 g, 15.25 mmol) at room temperature under N₂ atmosphere. After being stirred for 2 h, the solvent was removed under reduced pressure. The residue was purified by silica gel column chromatography (hexanes:EtOAc=3:1 to 1:1 v/v) to give compound 32 (1.15 g, 2.93 mmol) in 96% yield. ¹H NMR (400 MHz, CDCl₃) δ 7.42-7.28 (m, 10H), 4.87 (d, J=11.2 Hz, 1H), 4.70-4.66 (m, 1H), 4.67 (d, J=5.2 Hz, 1H), 4.62-4.52 (m, 3H), 4.37 (d, J=5.2 Hz, 1H), 4.20-3.82 (m, 2H); ¹³C NMR (100 MHz, CDCl₃) δ 170.4, 137.5, 136.6, 128.6, 128.4, 128.3, 128.0, 127.9, 80.6, 75.7, 74.6, 73.8, 67.6, 44.5.

### ((3S,5R)-4-(benzyloxy)-5-((benzyloxy)methyl)-3-bromo-3-fluorodihydrofuran-2(3H)-one (33)

To a solution of compound 32 (0.20 g, 0.54 mmol) and NFSi (0.17 g, 0.54 mmol) in 2.0 mL of anhydrous THF was added LiHMDS (0.54 mL, 0.54 mmol, 1.0 M in THF) dropwise at −78° C. under Ar atmosphere. After being stirred for 1 h at the same temperature, the reaction mixture was treated with 0.2 mL of saturated NH₄Cl (aq) and stirred for 10 min. The resulting slurry was poured into saturated cold NH₄Cl (aq) (5.0 mL) and EtOAc (15 mL). The organic layer was separated and the aqueous layer was washed with EtOAc (10 mL×2). The collected organic layer was washed with brine (10 mL), dried over Na₂SO₄ and filtered. The filtrate was concentrated under reduced pressure and the residue was purified by silica gel column chromatography (hexanes:EtOAc=10:1 to 3:1 v/v) to give compound 33 (0.14 g, 0.34 mmol) in 63% yield. ¹H NMR (400 MHz, CDCl₃) δ 7.44-7.31 (m, 10H), 4.97 (d, J=12.0 Hz, 1H), 4.87-4.83 (m, 1H), 4.65 (d, J=12.0 Hz, 1H), 4.63-4.53 (m, 3H), 3.89 (dd, J=4.0, 11.2 Hz, 1H), 3.89 (dd, J=7.2, 11.2 Hz, 1H); ¹³C NMR (100 MHz, CDCl₃) δ 165.0, 137.3, 135.7, 128.7, 128.6, 128.5, 128.3, 128.0, 127.8, 78.4, 78.0, 77.8, 73.7, 73.6, 67.1.

### Example 7

### Method 4 2-Cl Up, 2-F Down

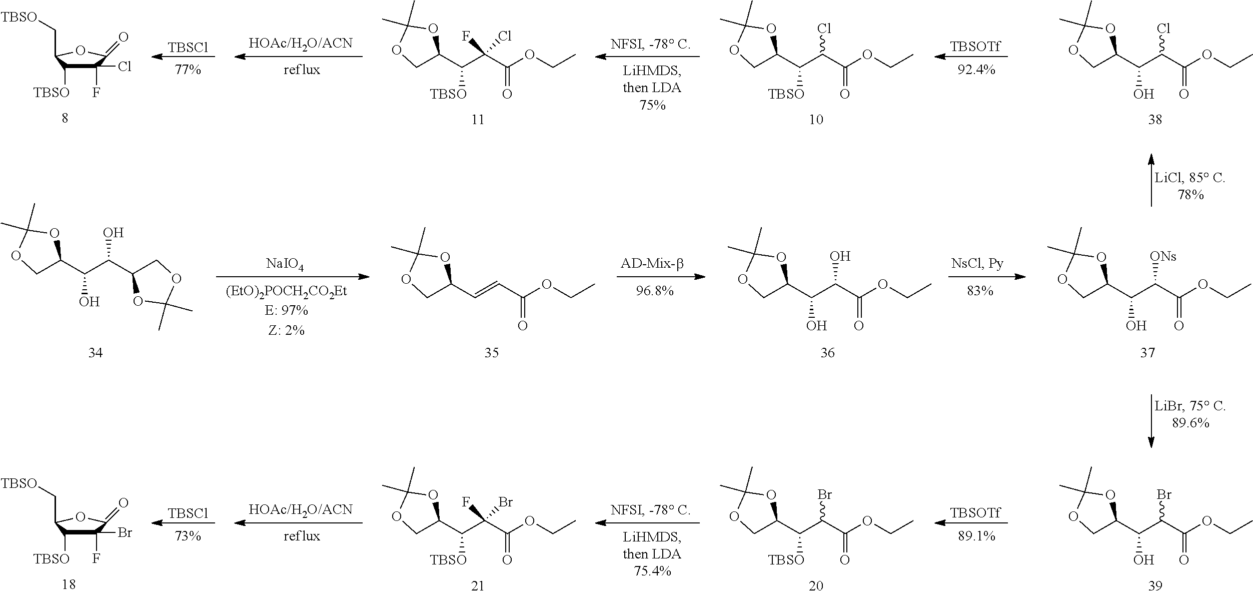

### Ethyl (S, E)-3-(2,2-dimethyl-1,3-dioxolan-4-yl)acrylate (35)

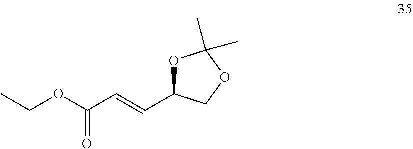

To a slurry of 18.9 g (72 mmol) of 1,2:5,6-di-O-isopropylidene-D-mannitol (34) in 150 mL of 5% aqueous NaHCO₃ at 0° C. was added dropwise a solution of 18.9 g (88.5 mmol) of NaIO₄ in 150 mL of water. The bath was removed and the mixture was stirred for 1 h. The mixture was cooled to 0° C. followed by the addition of triethyl α-phosphonoacetate (67.8 g, 300 mmol) and 450 mL of 6 M K₂CO₃. The reaction mixture was allowed to warm to room temperature and was stirred for 24 h. The reaction mixture was extracted four times with CH₂Cl₂ and the combined extracts were dried over Na₂SO₄, filtered and concentrated under reduced pressure. The residue was purified by Combiflash® with hexane/50% ether-hexane gradient afforded 28.0 g (97%) of E-isomer (35) and 0.7 g (2.4%) of Z-isomer.

¹H NMR (400 MHz, CDCl₃) δ 6.86 (dd, J=15.6, 5.6 Hz, 1H), 6.08 (dd, J=15.6, 0.8 Hz, 1H), 4.64 (m, 1H), 4.17 (q, J=7.2 Hz, 2H), 4.15 (m, 1H), 3.66 (dd, J=8.2, 7.2 Hz, 1H), 1.43 (s, 3H), 1.39 (s, 3H), 1.27 (t, J=7.2 Hz, 3H). ¹³C NMR (101 MHz, CDCl₃) δ 165.92, 144.56, 122.36, 110.09, 74.87, 68.73, 60.49, 26.38, 25.66, 14.13.

### Ethyl (2R,3S)-3-((R)-2,2-dimethyl-1,3-dioxolan-4-yl)-2,3-dihydroxypropanoate (36)

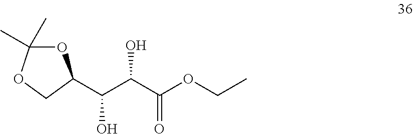 [SMILES: CCOC(=O)[C@@H](O)[C@H](O)[C@H]1COC(C)(C)O1]

To a solution of t-BuOH (150 mL) and H₂O (150 mL) was added AD-mix-β (42.0 g) at room temperature. When a clear two phase was formed (down phase is a pale yellow), methanesulfonamide (2.85 g, 30.0 mmol) was added. The mixture was cooled to 0° C. When the solution became a suspension, compound 35 (6.0 g, 30.0 mmol) was added. The reaction mixture was stirred at 4° C. for 24 h. Na₂S₂O₃ (45 g) was added at 4° C., and the mixture was stirred from 4° C. to room temperature for 60 min. Ethyl acetate (200 mL) was added and the water layer separated. The water phase was extracted with ethyl acetate (60 mL×3). The extracts were dried over Na₂SO₄, and concentrated under vacuum. The residue was purified by silica gel column chromatography (0 to 50% EtOAc in hexane gradient) to give 36 (6.8 g, 96.8%) as a colorless oil.

¹H NMR (400 MHz, CDCl₃) δ 4.43 (d, J=4.6 Hz, 1H), 4.32 (q, J=7.2 Hz, 2H), 4.15-4.04 (m, 3H), 3.88 (m, 1H), 3.14 (m, 1H), 2.27-2.23 (m, 1H), 1.45 (s, 3H), 1.37 (s, 3H), 1.33 (t, J=7.2 Hz, 3H). ¹³C NMR (101 MHz, CDCl₃) δ 173.42, 109.38, 75.20, 72.97, 70.40, 66.77, 62.13, 26.82, 25.13, 14.05.

### Ethyl (2R,3R)-3-((R)-2,2-dimethyl-1,3-dioxolan-4-yl)-3-hydroxy-2-(((4-nitrophenyl) sulfonyl) oxy)propanoate (37)

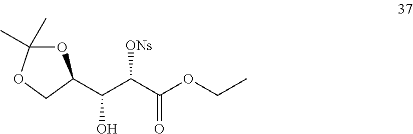

To a stirred solution of 36 (6.8 g, 29.06 mmol) in pyridine (125 mL) at 0° C. was added NsCl (7.4 g, 33 mmol). After being stirred at 4° C. for 18 h, the mixture was quenched with water (5 mL) at 4° C., treated with Et₂O (500 mL), washed with pre-cooled 1M aq. KHSO₄ (100 mL×3) and saturated brine (100 mL). The organic layer was dried over Na₂SO₄, and concentrated under vacuum. The residue was purified by silica gel column chromatography (0 to 60% EtOAc in hexane gradient) to give 37 (9.9 g, 83%) as a pale yellow oil.

¹H NMR (400 MHz, CDCl₃) δ 8.38 (d, J=7.0 Hz, 2H), 8.20 (d, J=7.0 Hz, 2H), 5.27 (d, J=2.0 Hz, 1H), 4.25 (q, J=7.1 Hz, 2H), 4.07-3.98 (m, 3H), 3.85-3.81 (m, 1H), 2.54 (brs, 1H), 1.37 (s, 3H), 1.28 (t, J=7.1 Hz, 3H), 1.20 (s, 3H). ¹³C NMR (101 MHz, CDCl₃) δ 166.98, 150.83, 141.94, 129.60, 124.16, 109.86, 78.63, 74.09, 72.76, 66.63, 62.64, 26.82, 24.88, 13.98.

### Ethyl (3R)-2-chloro-3-((R)-2,2-dimethyl-1,3-dioxolan-4-yl)-3-hydroxypropanoate (38)

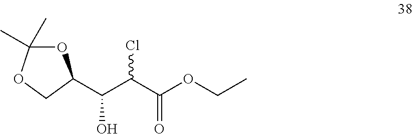 [SMILES: CCOC(=O)C(Cl)[C@H](O)[C@H]1COC(C)(C)O1]

To a stirred solution of 37 (4.8 g, 11.4 mmol) in DMF (60 mL) was added LiCl (970 mg, 22.8 mmol). The mixture was heated to 85° C. and stirred for 14 h. After the mixture was treated with Et₂O at 0° C., the ethereal solution was washed with pre-cooled 1M aq. KHSO₄ (50 mL×3) and saturated brine (50 mL), dried over Na₂SO₄, and concentrated under vacuum. The residue was purified by silica gel column chromatography (0 to 35% ethyl ether in hexane) to give 38 (2.25 g, 78%) as a colorless oil.

¹H NMR (400 MHz, CDCl₃) δ 4.73 (d, J=1.8 Hz, 0.5H), 4.5 (d, J=4.1 Hz, 0.5H), 4.31 (q, J=7.1 Hz, 2H), 4.20-4.01 (m, 3H), 2.96 (d, J=7.3 Hz, 0.5H), 2.73 (d, J=6.4 Hz, 0.5H), 1.62-1.32 (m, 9H).

In an alternate procedure, the free hydroxyl group in 37 is protected before the leaving group is displaced with the halide ion.

### Ethyl (3R)-3-((tert-butyldimethylsilyl)oxy)-2-chloro-3-((R)-2,2-dimethyl-1,3-dioxolan-4-yl)propanoate (10)

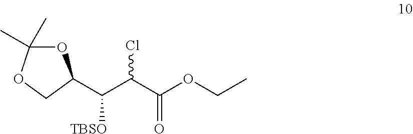 [SMILES: CCOC(=O)C(Cl)[C@H](O[Si](C)(C)C(C)(C)C)[C@H]1COC(C)(C)O1]

To a solution of 38 (3.0 g, 11.8 mmol) in DCM was added 2,6-lutidine (4.1 mL, 35.4 mmol) at 0° C. The mixture was added TBSOTf (4.1 mL, 17.7 mmol) dropwise at 0° C. The resulting mixture was stirred at room temperature for 3 h for the completion. The reaction was quenched with pre-cooled 1N HCl (30 mL) at 0° C., extracted with DCM (50 mL×3), washed with water (50 mL), brine (50 mL), dried over Na₂SO₄ and concentrated in vacuum. The residue was purified by silica gel column chromatography (0 to 20% ethyl acetate in hexane) to give 10 (4.0 g, 92.4%) as a colorless oil.

¹H NMR (400 MHz, CDCl₃) δ 4.65 (d, J=2.5 Hz, 0.5H), 4.59 (d, J=2.64 Hz, 0.5H), 4.30-4.17 (m, 4H), 4.09-4.04 (m, 1H), 3.92-3.88 (m, 1H), 1.43 (s, 1.5H), 1.39 (s, 1.5H), 1.34-1.32 (m, 6H), 0.89 (s, 4.5H), 0.87 (s, 4.5H), 0.18 (s, 1.5H), 0.14 (s, 1.5H), 0.11 (s, 1.5H), 0.04 (s, 1.5H).

### Ethyl (2S,3R)-3-((tert-butyldimethylsilyl)oxy)-2-chloro-3-((R)-2,2-dimethyl-1,3-dioxolan-4-yl)-2-fluoropropanoate (11)

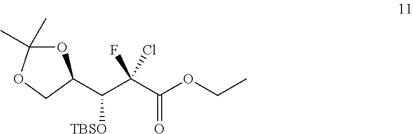 [SMILES: CCOC(=O)[C@@](F)(Cl)[C@H](O[Si](C)(C)C(C)(C)C)[C@H]1COC(C)(C)O1]

To a solution of 10 (3.95 g, 10.7 mmol) and NSFI (5.08 g, 16.1 mmol) in THF (50 mL) was added LiHMDS (16.1 mL, 16.1 mmol) dropwise at −78° C. The mixture was stirred at −78° C. for 1 h, and LDA solution (3.5 mL, 3.5 mmol) was added dropwise at this temperature. The reaction mixture was then stirred from −78° C. to −10° C. for completion (1h). The reaction was quenched with saturated NH₄Cl (50 mL) and ethyl acetate 200 mL) at −78° C. The organic layer was washed with saturated NH₄Cl (50 mL×2), water (50 mL), brine (50 mL), dried over Na₂SO₄ and concentrated in vacuum. The residue was purified by silica gel column chromatography (0 to 20% ethyl acetate in hexane) to give 11 (3.1 g, 75%) as a colorless oil.

¹H NMR (400 MHz, CDCl₃) δ 4.37-4.26 (m, 4H), 4.04 (dd, J=8.2, 6.6 Hz, 1H), 3.92 (dd, J=8.1, 6.4 Hz, 1H), 1.41 (s, 3H), 1.36 (t, J=7.1 Hz, 3H), 1.32 (s, 3H), 0.92 (s, 9H), 0.19 (s, 3H), 0.16 (s, 3H); ¹⁹F NMR (377 MHz, CDCl₃) δ −127.14 (d, J=18.1 Hz).

### (3S,4R,5R)-4-((tert-butyldimethylsilyl)oxy)-5-(((tert-butyldimethylsilyl)oxy)methyl)-3-chloro-3-fluorodihydrofuran-2(3H)-one (8)

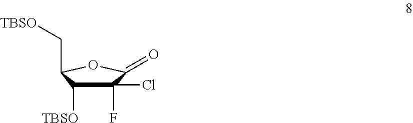

A solution of 11 (2.9 g, 7.5 mmol) in AcOH/H₂O/ACN (4:1:10, 15 mL) was refluxed at 100° C. for 2.5 h (monitored by TLC). The solvent was removed under reduced pressure and co-evaporated with toluene (10 mL×3). The residue was charged with imidazole (1.22 g, 18 mmol), TBSCl (2.26 g, 15 mmol) and DMF (20 mL) at 0° C. The reaction mixture was stirred at room temperature overnight for completion. The reaction was quenched with water and extracted with ethyl ether (200 mL). The organic layer was washed with water (30 mL×3), saturated NH₄Cl (50 mL) and brine (50 mL), dried over Na₂SO₄ and concentrated in vacuum. The residue was purified by silica gel column chromatography (0 to 8% ethyl acetate in hexane) to give 8 (2.4 g, 77%) as a colorless oil.

¹H NMR (400 MHz, CDCl₃) δ 4.59 (dd, J=11.9, 5.8 Hz, 1H), 4.37-4.32 (m, 1H), 3.98 (dd, J=12.1, 3.9 Hz, 1H), 3.85 (dd, J=12.1, 3.1 Hz, 1H), 0.93 (s, 9H), 0.90 (s, 9H), 0.22 (s, 3H), 0.17 (s, 3H), 0.10 (s, 3H), 0.09 (s, 3H); ¹⁹F NMR (377 MHz, CDCl₃) δ −134.66 (d, J=12.0 Hz); ¹³C NMR (101 MHz, CDCl₃) δ 165.11 (d, J=26.26 Hz), 102.06 (d, J=260.6 Hz), 83.60, 74.06 (d, J=16.2 Hz), 59.52, 25.70, 25.50, 18.20, 18.01, −4.45, −5.21, −5.48, −5.52.

### Example 8

### Method 4, 2-Br,2-F

### Ethyl (3R)-2-bromo-3-((R)-2,2-dimethyl-1,3-dioxolan-4-yl)-3-hydroxypropanoate (39)

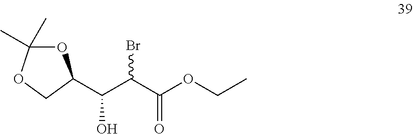 [SMILES: CCOC(=O)C(Br)[C@H](O)[C@H]1COC(C)(C)O1]

To a stirred solution of 37 (5.98 g, 14.3 mmol) in THF (70 mL) was added LiBr (6.4 g, 73.5 mmol). The mixture was heated to 75° C. and stirred for overnight. After the mixture was treated with Et₂O (300 mL) at 0° C., the ethereal solution was washed with pre-cooled 1M aq. KHSO₄ (50 mL x 3) and saturated brine (50 mL), dried over Na₂SO₄, and concentrated under vacuum. The residue was purified by silica gel column chromatography (0 to 35% ethyl ether in hexane) to give 39 (3.8 g, 89.6%) as a colorless oil.

¹H NMR (400 MHz, CDCl₃) δ 4.66 (d, J=2.2 Hz, 0.5H), 4.5 (d, J=4.4 Hz, 0.5H), 4.30-4.23 (m, 2H), 4.16-4.01 (m, 3H), 3.93-3.71 (m, 1H), 3.37 (d, J=8.1 Hz, 0.5H), 3.22 (d, J=4.0 Hz, 0.5H), 1.41 (s, 1.5H), 1.40 (s, 1.5H), 1.34 (s, 3H), 1.32 (t, J=7.1 Hz, 1.5H), 1.31 (t, J=7.1 Hz, 1.5H).

### Ethyl (3R)-3-((tert-butyldimethylsilyl)oxy)-2-bromo-3-((R)-2,2-dimethyl-1,3-dioxolan-4-yl)propanoate (20)

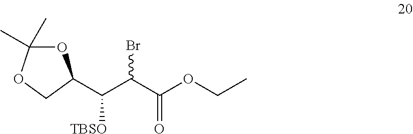 [SMILES: CCOC(=O)C(Br)[C@H](O[Si](C)(C)C(C)(C)C)[C@H]1COC(C)(C)O1]

To a solution of 39 (3.8 g, 12.8 mmol) in DCM (60 mL) was added 2,6-lutidine (4.5 mL, 38.8 mmol) at 0° C. The mixture was added TBSOTf (4.5 mL, 19.4 mmol) dropwise at 0° C. The resulting mixture was stirred at room temperature for 3 h. The reaction was quenched with pre-cooled 1N HCl (30 mL) at 0° C., extracted with DCM (50 mL×3), washed with water (50 mL), brine (50 mL), dried over Na₂SO₄ and concentrated in vacuo. The residue was purified by silica gel column chromatography (0 to 20% ethyl acetate in hexane) to give 20 (4.58 g, 87.1%) as a colorless oil.

¹H NMR (400 MHz, CDCl₃) δ 4.65 (d, J=2.8 Hz, 0.5H), 4.90 (d, J=3.8 Hz, 0.5H), 4.34-4.11 (m, 4H), 4.08-4.01 (m, 1H), 3.91-3.87 (m, 1H), 1.41 (s, 1.5H), 1.39 (s, 1.5H), 1.33-1.30 (m, 6H), 0.89 (s, 4.5H), 0.87 (s, 4.5H), 0.16 (s, 1.5H), 0.13 (s, 1.5H), 0.11 (s, 1.5H), 0.05 (s, 1.5H).

### Ethyl (2S,3R)-3-((tert-butyldimethylsilyl)oxy)-2-bromo-3-((R)-2,2-dimethyl-1,3-dioxolan-4-yl)-2-fluoropropanoate (21)

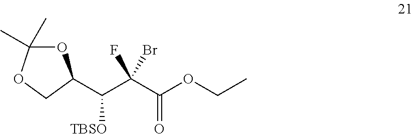 [SMILES: CCOC(=O)[C@@](F)(Br)[C@H](O[Si](C)(C)C(C)(C)C)[C@H]1COC(C)(C)O1]

To a solution of 20 (4.45 g, 10.87 mmol) and NSFI (5.12 g, 16.23 mmol) in THF (60 mL) was added LiHMDS (18.5 mL, 18.5 mmol) dropwise at −78° C. The mixture was stirred at −78° C. for 1 h, and LDA solution (2 mL, 2 mmol) was added dropwise at this temperature. The reaction mixture was then stirred from −78° C. to −10° C. for completion (1 h). The reaction was quenched with saturated NH₄Cl (50 mL) and ethyl acetate 200 mL) at −78° C. The organic layer was washed with saturated NH₄Cl (50 mL×2), water (50 mL), brine (50 mL), dried over Na₂SO₄ and concentrated in vacuum. The residue was purified by silica gel column chromatography (0 to 20% ethyl acetate in hexane) to give 21 (3.5 g, 75.4%) as a colorless oil.

¹H NMR (400 MHz, CDCl₃) δ 4.45-4.25 (m, 4H), 4.03 (dd, J=8.2, 6.6 Hz, 1H), 3.92 (dd, J=8.1, 6.5 Hz, 1H), 1.41 (s, 3H), 1.36 (t, J=7.1 Hz, 3H), 1.32 (s, 3H), 0.93 (s, 9H), 0.22 (s, 3H), 0.17 (s, 3H); ¹⁹F NMR (377 MHz, CDCl₃) δ −126.16 (d, J=18.0 Hz); ¹³C NMR (101 MHz, CDCl₃) δ 165.24 (d, J=26.36 Hz), 108.77, 98.75 (d, J=271.84 Hz), 77.76 (d, J=20.42 Hz), 75.60 (d, J=4.16 Hz), 65.48, 63.02, 25.89, 25.86, 24.50, 18.33, 13.78, −3.91, −4.08, −4.10.

### (3S,4R,5R)-4-((tert-butyldimethylsilyl)oxy)-5-(((tert-butyldimethylsilyl)oxy)methyl)-3-bromo-3-fluorodihydrofuran-2(3H)-one (18)

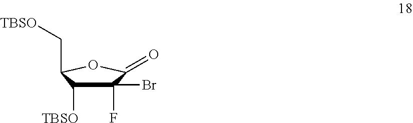

A solution of 21 (1.54 g, 3.75 mmol) in AcOH/H₂O/ACN (4:1:10, 8 mL) was refluxed at 100° C. for 2.5 h (monitored by TLC). The solvent was removed under reduced pressure and co-evaporated with toluene (10 mL×3). The residue was charged with imidazole (456 mg, 6.7 mmol), TBSCl (845 mg, 5.6 mmol) and DMF (10 mL) at 0° C. The reaction mixture was stirred at room temperature overnight. The reaction was quenched with water and extracted with ethyl ether (150 mL). The organic layer was washed with water (30 mL×3), saturated NH₄Cl (50 mL) and brine (50 mL), dried over Na₂SO₄ and concentrated in vacuum. The residue was purified by silica gel column chromatography (0 to 8% ethyl acetate in hexane) to give 18 (1.2 g, 73%) as a colorless oil.

¹H NMR (400 MHz, CDCl₃) δ 4.73 (dd, J=8.6, 4.3 Hz, 1H), 4.41-4.37 (m, 1H), 3.99 (dd, J=11.7, 5.3 Hz, 1H), 3.90 (dd, J=11.7, 3.9 Hz, 1H), 0.93 (s, 9H), 0.90 (s, 9H), 0.22 (s, 3H), 0.18 (s, 3H), 0.10 (s, 3H), 0.09 (s, 3H); ¹⁹F NMR (377 MHz, CDCl₃) δ −136.63 (d, J=8.6 Hz); ¹³C NMR (101 MHz, CDCl₃) δ 165.86 (d, J=25.5 Hz), 94.05 (d, J=275.1 Hz), 84.73, 75.13 (d, J=15.4 Hz), 60.12, 25.74, 25.53, 18.23, 18.05, −4.38, −5.18, −5.46, −5.47.

### Example 9

### Example Synthesis of Compound 18

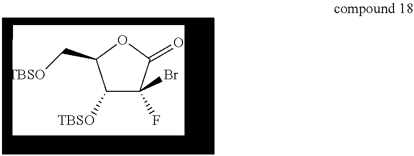 [SMILES: CC(C)(C)[Si](C)(C)OC[C@H]1OC(=O)[C@@](F)(Br)[C@@H]1O[Si](C)(C)C(C)(C)C]

The synthetic route is shown below:

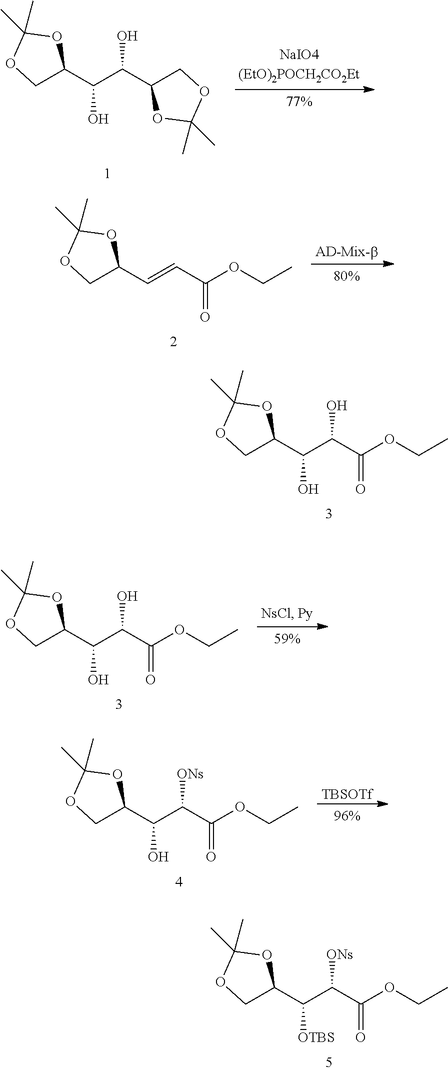

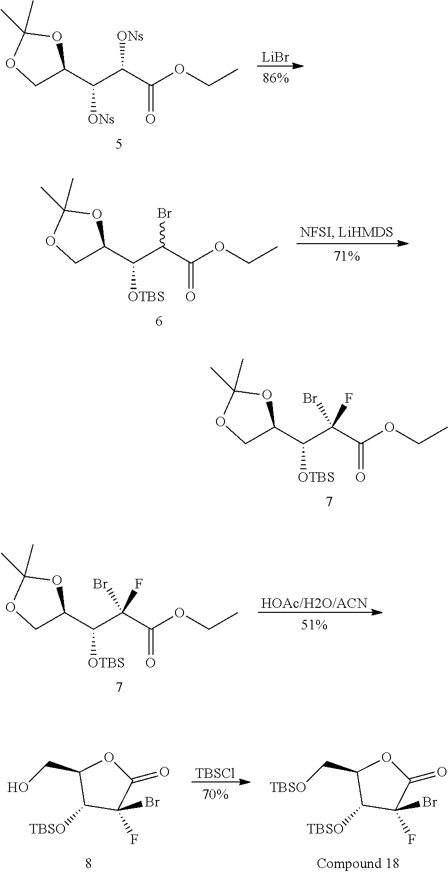

This route differs slightly from the route outlined in Example 7, in that the order of the protection step (of the OH moiety in Compound 4) and the displacement of the nosylate group by bromide ions is reversed.

### Experimental:

### Scale: 5.72 mol

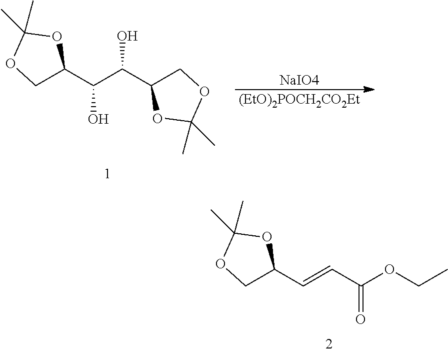

### Reagents:

| | | | Density at | | | | |
|---|---|---|---|---|---|---|---|
| | | | 25° C. | Amount | | | |
| | Reagent/reactant/solvent | MW | (g/mL) | (kg) | Mol | Equiv. | X |
| 1 | Compound 1 | 262.3 | / | 1.5 kg | 5.72 | 1.00 | 1.00 |
| | | | | | | | (wt) |
| 2 | ethyl 2- | 224.19 | / | 5.39 | 24.02 | 4.2 | 3.6 |
| | diethoxyphosphorylacetate | | | | | | (wt) |
| 3 | NaIO₄ | 213.89 | / | 1.18 | 7.04 | 1.23 | 0.79 |
| | | | | | | | (wt) |
| 4 | H₂O | / | / | 12 L | / | / | 8v |

### Procedure:

The reactor was set up, and NaHCO₃ (5%) (12.00 L) was charged into the reactor at 24° C. Compound 1 (1.50 kg, 5.72 mol, 1.00 eq) was then charged into the reactor at 0° C. A solution of NaIO₄ (1.50 kg, 7.04 mol, 390 mL, 1.23 eq) in H₂O (12.00 L) was then added dropwise into the reactor at 0° C. The mixture was stirred at 24° C. for 1 hr.

Ethyl 2-diethoxyphosphorylacetate (5.39 kg, 24.0 mol, 4.77 L, 4.20 eq) was then charged into the reactor at 0° C., and K₂CO₃(6M) (12.00 L) was added dropwise into the reactor at 0° C.

The reaction mixture was then allowed to warm to 24° C. and was stirred for 17 h.

A TLC (Petroleum ether: Ethyl acetate=2/1) showed the reaction was finished and one major new spot with lower polarity was detected. The reaction mixture was extracted with methyl t-butyl ether (MTBE) (8 L×2), and the organic layer was dried over Na₂SO₄, filtered and concentrated under reduced pressure. The residue was purified by column chromatography (SiO₂, Petroleum ether/Ethyl acetate=100/1 to 20:1) to yield Compound 2 (1.81 kg, 8.80 mol, 77% yield, 97.3% purity) as a light yellow oil.

¹H NMR: ET11581-3-P1A1 400 MHz CDCl₃: δ=6.86 (dd, J=5.5, 15.7 Hz, 1H), 6.08 (dd, J=1.4, 15.5 Hz, 1H), 4.68-4.62 (m, 1H), 4.24-4.04 (m, 3H), 3.66 (dd, J=7.1, 8.2 Hz, 1H), 1.46-1.37 (m, 6H), 1.28 (t, J=7.2 Hz, 3H)

3 kg of Compound 1 was converted to Compound 2 in two batches (each 1.5 kg), with molar yields of 75 and 77%. The Z isomer was difficult to separate from the E isomer, which led to the relatively low yield

### Preparation of Compound 3

### Scale: 3.5 mol

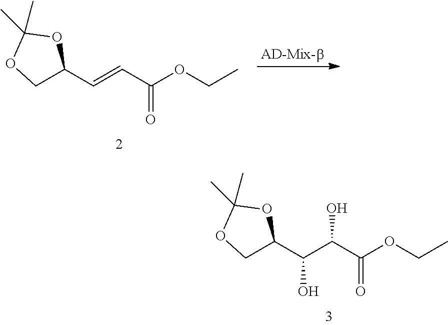 [SMILES: CCOC(=O)/C=C/[C@H]1COC(C)(C)O1 ; CCOC(=O)[C@@H](O)[C@H](O)[C@H]1COC(C)(C)O1]

### Reagents:

| | | | Density | | | | |
|---|---|---|---|---|---|---|---|
| | | | at 25° C. | Amount | | | |
| | Reagent/ reactant/solvent | MW | (g/mL) | (g) | mol | Equiv. | X (wt) |
| 1 | Compound 2 | 200.23 | / | 700 | 3.5 | 1.00 | 1.00 |
| 2 | AD-Mix-β | / | / | 4900 | / | N/A | 7.00 |
| 3. | methanesulfonamide | 95.12 | / | 333 | 3.5 | 1 | 0.47 |
| 4. | H₂O | / | 1 | 17.5 (L) | / | / | 25 |
| 5. | t-BuOH | / | 0.78 | 17.5 (L) | / | / | 19.5 |

### Procedure:

H₂O (17.50 L) and t-BuOH (17.50 L) were charged into a reactor. AD-mix-β (4.9 kg) was charged into the reactor, and stirred at 20° C. for 0.5 hr. When a clear two phase mixture was formed (the denser phase is a pale yellow), methanesulfonamide (333 g, 3.50 mol, 1.00 eq) was charged into the reactor.

The reaction mixture was cooled to 0° C., and Compound 2 (700 g, 3.50 mol, 1.00 eq) was charged into the reactor. The mixture was stirred at 0-5° C. for 60 hr. A TLC (Petroleum ether: Ethyl acetate=2/1) showed the reaction was finished. Na₂S₂O₃ (5.25 kg) was charged into the reactor, and the reaction mixture was stirred at 0-5° C. for 1 hr. EtOAc (10 L) was charged into the reactor, and the water layer was separated. The water phase was washed with EA (5 L). The organic layer was washed with brine (5 L), dried over by Na₂SO₄, and concentrated, to yield Compound 3 (657 g) as a yellow oil, which was used in the next step without further purification.

¹H NMR (400 MHz, Chloroform-d) δ=4.38 (s, 1H), 4.24 (q, J=7.1 Hz, 2H), 4.12-3.96 (m, 3H), 3.88-3.77 (m, 1H), 3.37 (br s, 1H), 2.65 (br d, J=9.0 Hz, 1H), 1.46-1.37 (m, 3H), 1.36-1.19 (m, 6H)

3.28 kg of Compound 2 was converted to Compound 3 in five batches (480 g, 700 g*4), with conditions and product yields shown in the table below.

| | Conditions | | Product | | |
|---|---|---|---|---|---|
| Cpd. 2 | Temp. | Time | Weight | Purity | Yield |
| (g) | (° C.) | (hrs) | (kg) | (%) | (%) |
| 700 | 0~5 | 60 | 657 | N/A | N/A |
| 700 | 0~5 | 60 | 583 | N/A | N/A |
| 480 | 0 | 72 | 448 | 100 | 80 |
| 700*2 | 4 | 24 | 1700 | crude | N/A |

### Notes:

The AD-mix-β was made in house.
Slightly raising the reaction temperature could speed up the reaction (reaction time 72 hrs at 0° C., 40 hrs at 2° C., 24 hrs at 4° C.)
Crude compound 3 can be used in the next step without purification.

### Preparation of Compound 4

### Scale: 6.66

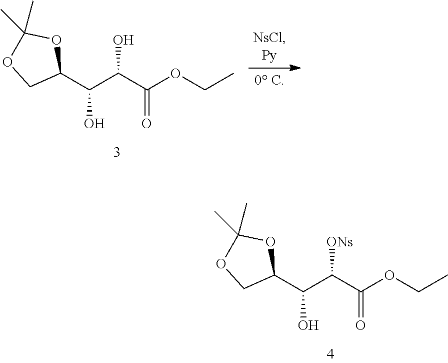

### Reagents:

| | | | Density at | | | | |
|---|---|---|---|---|---|---|---|
| | | | 25° C. | Amount | | | |
| | Reagent/reactant/solvent | MW | (g/mL) | (kg) | Mol | Equiv. | X |
| 1 | Compound 3 | 234.5 | / | 1.56 | 6.66 | 1.00 | 1.00 |
| | | | | | | | (wt) |
| 2 | 4-nitrobenzenesulfonyl | 221.62 | / | 1.59 | 7.19 | 1.08 | 1.02 |
| | chloride | | | | | | (wt) |
| 3 | Py | / | 0.98 g/ml | 18 L | 0.50 | 0.5 | 11.5 v |

### Procedure:

The reactor was set up, and pyridine (Py, 18.0 L) was charged into the reactor at 24° C. Compound 3 (1.56 kg, 6.66 mol, 1.00 eq) was charged into the reactor at 24° C. 4-nitrobenzenesulfonyl chloride (1.59 kg, 7.19 mol, 1.08 eq) was then charged into the reactor at 0-5° C. The mixture was stirred at 0-5° C. for 3-4 hr. TLC (Petroleum ether:Ethyl acetate=2/1) showed the reaction was finished and one major new spot with lower polarity was detected. The mixture was quenched with water (36 L) at 4° C., extracted with MTBE (20 L×2), washed with pre-cooled 1M aq. KHSO₄ (30 L×4), aq.NaHCO₃ (10 L), and saturated brine (10 L). The organic layer was dried over Mg₂SO₄, and filtered, concentrated under reduced pressure and co-evaporated with toluene (5 L×2) to remove residual pyridine and obtain a crude product.(3.2 kg, crude). The crude product was dissolved in EtOAc (3.2 L), and, with stirring, petroleum ether (9.6 L) was added. This resulted in the precipitation of a solid. The solid was filtered and dried to give 1.64 kg product.

The mother liquor (2.2 kg) was purified by colummn chromatography (SiO₂, petroleum ether/ethyl acetate=10/1 to 5:1) to give 1.5 kg (crude) product. The 1.5 kg crude product was washed with petroleum ether/EtOAc (2.6 L/428 ml) and filtered, and the solid was dried under vacuum to give the product (1.2 kg). The filter was concentrated in vacuum to give the crude product (440 g).

The crude product (440 g) was purified by column chromatography (SiO₂, petroleum ether/ethyl acetate=10/1 to 5:1) to give the product (383 g). Compound 4 (3.22 kg) was obtained as a yellow solid.

¹H NMR: 400 MHz CDCl₃: δ=8.42-8.36 (m, J=8.8 Hz, 2H), 8.24-8.16 (m, J=8.8 Hz, 2H), 5.27 (d, J=1.8 Hz, 1H), 4.25 (q, J=7.0 Hz, 2H), 4.07-3.94 (m, 3H), 3.88-3.79 (m, 1H), 2.39 (br s, 1H), 1.37 (s, 3H), 1.29 (t, J=7.2 Hz, 3H), 1.20 (s, 3H) 3.05 kg of Compound 3 was converted to Compound 4 in two batches (1.56 kg and 1.49 kg).

| | Conditions | | Product | | |
|---|---|---|---|---|---|
| Cpd. 3 | Temp. | Time | Weight | Purity | Yield |
| (kg) | (° C.) | (hrs) | (kg) | (%) | (%) |
| 1.56 | 0~24 | 3~4 | 3.22 | 95 | 59 |
| 1.49 | 0~24 | 3~4 | | | |

### Note:

The product can be purified by re-crystallization, but this tends to lead to low yields.
The product is unstable at 20° C., and should be stored at around −10 to around −20° C.

### Preparation of Compound 5

### Scale: 3.81 kg

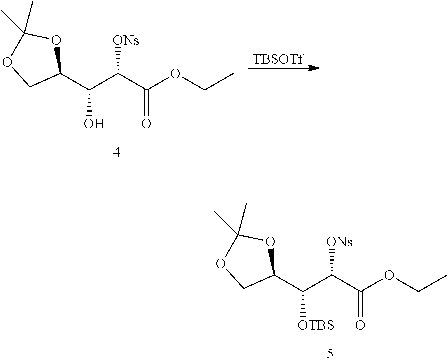

### Reagents:

| | | | Density at | | | | |
|---|---|---|---|---|---|---|---|
| | | | 25° C. | | | | |
| | Reagent/reactant/solvent | MW | (g/mL) | Amount (Kg) | mol | Equiv. | X |
| 1 | Compound 4 | 419.4 | / | 1.6 | 3.81 | 1.00 | 1.00 |
| | | | | | | | (wt) |
| 2 | TBSOTF | 264.34 | / | 2.01 | 7.62 | 2 | 1.25 |
| | | | | | | | (wt) |
| 3 | 2,6-Lutidine | 107.15 | / | 1.63 | 15.24 | 4 | 1.0 |
| | | | | | | | (wt) |
| 3 | DCM (dichloromethane) | / | 1.32 | (12 L) | / | / | 7.5 |
| | | | | | | | (vol) |

### Procedure:

The reactor was set up, and dichloromethane (DCM) (12.0 L) was charged into the reactor at 24° C. Compound 4 (1.60 kg, 3.81 mol, 1.00 eq) was charged into the reactor at 24° C. 2,6-Lutidine (1.63 kg, 15.2 mol, 1.77 L, 4.00 eq) was charged into the reactor at 0-5° C. TBSOTf (2.01 kg, 7.62 mol, 1.75 L, 2.00 eq) was charged into the reactor at 0-5° C. The mixture was stirred at 24° C. for between 3and 4hours, at which time TLC (Petroleum ether:Ethyl acetate=2/1) showed the reaction was finished. The mixture was quenched with HCl (1N, 15 L) at 0-4° C., separated, the water phase was extracted with DCM (5 L).

The combined organic layer were washed with aq. NaHCO₃ (5 L), dried over Mg₂SO₄, filtered and concentrated under reduced pressure to give the crude product. The residue was purified by column chromatography (SiO₂, petroleum ether/ethyl acetate=100/1 to 10:1) to yield Compound 5 (2.00 kg, 3.67 mol, 96.40% yield, 98% purity) as a yellow solid.

¹H NMR: 400 MHz CDCl₃: δ=8.36 (d, J=9.2 Hz, 2H), 8.17 (d, J=8.8 Hz, 2H), 5.25 (d, J=1.8 Hz, 1H), 4.23-4.10 (m, 3H), 3.99 (dd, J=6.1, 7.9 Hz, 1H), 3.90-3.79 (m, 2H), 1.43-1.32 (m, 3H), 1.27-1.16 (m, 6H), 0.81 (s, 9H), 0.04 (s, 3H), 0.02 (s, 3H) 3.22 kg of Compound 4 was converted to Compound 5 in three batches (1.6 kg, 1.24 kg, 0.38 kg).

| | Conditions | | Product | | |
|---|---|---|---|---|---|
| Cpd. 1 | Temp. | Time | Weight | Purity | Yield |
| (kg) | (° C.) | (hrs) | (kg) | (%) | (%) |
| 1.6 | 0~34 | 3~4 | 2 | 98 | 96.4 |
| 1.24 | 0~34 | 3~4 | 1.58 | 97 | 96.4 |
| 0.38 | 0~34 | 3~4 | 0.25 | N/A | 51.9 |

Preparation of Compound 6 (Scale=3.6 mol)

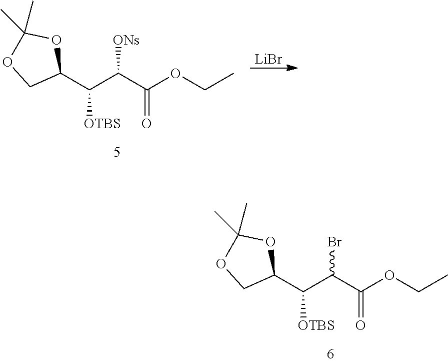

### Reagents:

| | Reagent/ | | density at | | | | |
|---|---|---|---|---|---|---|---|
| | reactant/ | | 25° C. | Amount | | | X |
| | solvent | MW | (g/mL) | (kg) | Mol | Equiv. | (wt) |
| 1 | Compound 5 | 533.6 | / | 1.92 | 3.6 | 1.00 | 1.00 |
| 2 | LiBr | 86.85 | / | 1.54 | 17.74 | 4.9 | 0.8 |
| 3 | DMF | | 0.95 | 12 (L) | / | / | 6 (V) |

### Procedure:

The reactor was set up, and DMF (12.0 L) was charged into the reactor at 24° C. LiBr (1.54 kg, 17.7 mol, 445 mL, 4.93 eq) was charged into the reactor at 24° C., and the temperature was raised to 55° C. Compound 5 (1.92 kg, 3.60 mol, 1.00 eq) was charged into the reactor at 30˜40° C. The mixture was stirred at 75˜80° C. for around 4-5 hr. TLC (Petroleum ether:Ethyl acetate=10/1) showed the reaction was finished.

The mixture was quenched with water (24 L) at 30° C., extracted with MTBE (5 L×3), and the combined organic phase were washed with saturated brine (5 L). The organic layer was dried over Na₂SO₄, and concentrated under reduced pressure to yield Compound 6 (1.20 kg, 2.92 mol, 81.02% yield) as a yellow oil.

¹H NMR: 400 MHz CDCl_{3;} δ=4.48-4.65(m, 1H), 4.12-4.23 (m, 5H), 3.88-3.90 (m, 1H), 1.33-1.41 (m, 3H), 1.29-1.32 (m, 6H), 0.830.91 (m, 9H), 0.06-0.16 (m, 6H).

3.57 kg of Compound 5 was converted to Compound 6 in three batches (1.92 kg, 1.4 kg, 0.25 kg).

| | Conditions | | Product | | |
|---|---|---|---|---|---|
| Cpd. 2 | Temp. | Time | Weight | Purity | Yield |
| (kg) | (° C.) | (hrs) | (kg) | (%) | (%) |
| 1.92 | 75~80 | 4~5 | 1.2 | N/A | 81% |
| 1.4 | 75~80 | 4~5 | 1 | N/A | 92.78% |
| 0.25 | 75~80 | 4~5 | 0.11 | N/A | 56.62% |

### Note:

This reaction can be performed using commercial anhydrous LiBr without further vacuum drying.

### Preparation of Compound 7

### Scale: 2.92 mol

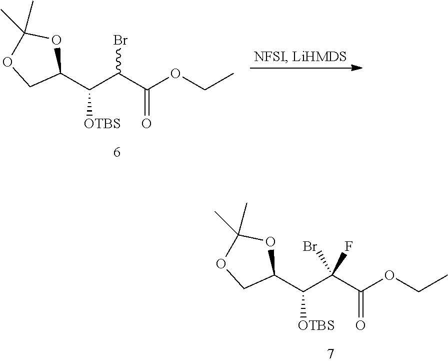

### Reagents:

| | Reagent/ | | density at | | | | |
|---|---|---|---|---|---|---|---|
| | reactant/ | | 25° C. | Amount | | | X |
| | solvent | MW | (g/mL) | (kg) | Mol | Equiv. | (wt) |
| 1 | Compound 6 | 411 | / | 1.2 | 2.92 | 1.00 | 1.00 |
| 2 | NFSI | 250.5 | / | 1.38 | 4.38 | 1.5 | 1.15 |
| | [133745- | | | | | | |
| | 75-2] | | | | | | |
| 3 | LiHMDS | 167.33 | 1M | 4.08 L | / | 1.4 | N/A |
| 4 | THF | / | anhydrous | 12 L | / | / | N/A |

### Procedure:

The reactor was set up, and THF (12 L) was charged into the reactor at 24° C. Compound 6 (1.20 kg, 2.92 mol, 1.00 eq) was charged into the reactor at 24° C. N-(benzenesulfonyl)-N-fluoro-benzenesulfonamide (1.38 kg, 4.38 mol, 1.50 eq) was charged into the reactor at 24° C. LiHMDS (1 M, 4.08 L, 1.40 eq) was added dropwise into the reactor at −65° C. The reaction mixture was stirred at −65° C. for 2 h, at which time ¹HNMR showed ˜15% of Reactant 1 was remained and ˜80% of desired compound was detected.

The reaction mixture was worked up with saturated aq.NH₄Cl (12 L). The mixture was extracted with MTBE (5 L×2), and the organic layer was washed with saturated aq.NaCl (5 L×3), dried over Na₂SO₄, filtered and concentrated under reduced pressure. The residue was purified by column chromatography (SiO₂, petroleum ether/ethyl acetate=1/0 to 50:1) to provide Compound 7 (890 g, 2.07 mol, 71% yield) as light yellow oil.

¹HNMR: δ=4.21-4.40 (m, 4H), 4.03-4.05 (m, 1H), 3.91-3.94 (m, 1H), 1.30-1.41 (m, 9H), 0.92 (s, 9H), 0.14-0.22 (m, 6H).

2.2 kg of 6 had been converted to 7 in two batches. (1.2 kg, 1 kg).

| | Conditions | | Product | | |
|---|---|---|---|---|---|
| Cpd. 6 | Temp. | Time | Weight | Purity | Yield |
| (kg) | (° C.) | (hrs) | (g) | (%) | (%) |
| 1.2 | −65 | 2 | 890 | / | 71 |
| 1 | −50~−65 | 2 | 750 | / | 72 |

### Note:

The purification is somewhat difficult, due to the relatively poor separation of compounds 6 and 7 on TLC. These compounds have substantially the same Rf value on TLC. However, it is possible to determine the endpoint of the reaction by ¹HNMR.

Preparation of Compound 8

### Scale: 3.73 mol

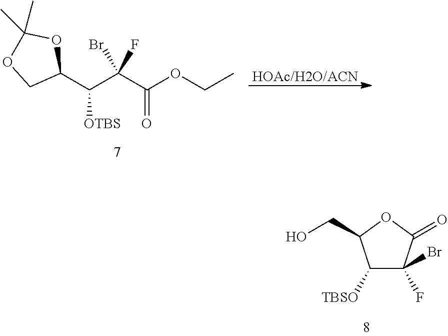 [SMILES: CCOC(=O)[C@@](F)(Br)[C@H](O[Si](C)(C)C(C)(C)C)[C@H]1COC(C)(C)O1 ; "HOAc/H2O/ACN" ; CC(C)(C)[Si](C)(C)O[C@@H]1[C@@H](CO)OC(=O)[C@@]1(F)Br]

### Reagents:

| | Reagent/ | | density at | | | | |
|---|---|---|---|---|---|---|---|
| | reactant/ | | 25° C. | Amount | | | X |
| | solvent | MW | (g/mL) | (kg) | Mol | Equiv. | (wt) |
| 1 | Compound 7 | 429 | / | 1.6 | 3.73 | 1.00 | 1.00 |
| 2 | HOAc/H₂O/ | / | / | 9.6 L | / | / | / |
| | CH₃CN | | | | | | |
| | (4/1/10) | | | | | | |

### Procedure:

The reactor was set up, and HOAc/H₂O/CH₃CN=4/1/10 (9.6 L) was charged into the reactor at 24° C. Compound 7(1.60 kg, 3.73 mol, 1.00 eq) was charged into the reactor at 24° C. The reaction mixture was refluxed in a 110° C. oil bath for 2.5 h, at which point TLC indicated the reaction was finished, and ˜60% of desired compound was detected.

The solvent was removed in reduced pressure and co-evaporated with toluene (1 L×2), and the residue was purified by column chromatography (SiO₂, petroleum ether/ethyl acetate=100/1 to 20:1) to yield Compound 8 (650 g, 1.89 mol, 51% yield) as a yellow oil.

¹HNMR: δ=4.65-4.71 (m, 1H), 4.42-4.48 (m, 1H), 4.02-4.06 (m, 1H), 3.87-3.91 (m, 1H), 0.93 (s, 9H), 0.24 (s, 3H), 0.19 (s, 3H).

1.6 kg of 7 had been converted to 8 in one batch.(1.6 kg).

| | Conditions | | Product | | |
|---|---|---|---|---|---|
| Cpd. 7 | Temp. | Time | Weight | Purity | Yield |
| (kg) | (° C.) | (hrs) | (g) | (%) | (%) |
| 1.6 | 110 | 2.5 | 650 | / | 51 |

### Preparation of Compound 18

### Scale: 1.6 mol

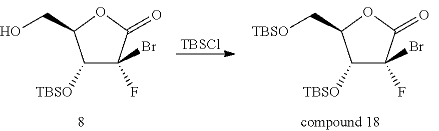

### Reagents:

| | | | density at | | | | |
|---|---|---|---|---|---|---|---|
| | Reagent/ | | 25° C. | Amount | | | X |
| | reactant/solvent | MW | (g/mL) | (g) | Mol | Equiv. | (wt) |
| 1 | Compound 8 | 343 | / | 550 | 1.6 | 1.00 | 1.00 |
| 2 | TBSCl | 150.7 | / | 362 | 2.4 | 1.50 | 0.66 |
| | (18162-48-6) | | | | | | |
| 3 | Imidazole | 68 | / | 218 | 3.2 | 2.00 | 0.40 |
| | (288-32-4) | | | | | | |
| 4 | DMF | / | / | 4.4 L | / | / | 8 V |

### Procedure:

The reactor was set up, and Compound 8 (550 g, 1.6 mol, 1.00 eq) was charged into the reactor. Imidazole (218 g, 3.2 mol, 1.00 eq) was charged into the reactor at 25° C. TBSCl (362 g, 2.4 mol) was charged into the reactor in portions. The reaction mixture was stirred at 25° C. for 1 h, at which time TLC indicated the reaction was finished, and one major new spot with lower polarity was detected.

The reaction was quenched with water (5 L) and the crude product was extracted with PE (5 L). The organic layer was washed with water (3 L), then saturated aqueous solution of NH4Cl (3 L), then brine(3 L), and then dried over Na₂SO₄, filtered, and concentrated under reduced pressure. The combined residues were purified by column chromatography (SiO₂, petroleum ether/ethyl acetate=1/0 to 100:1) to yield Compound 18 (600 g, 1.3 mol) obtained as a light yellow oil.

¹HNMR: δ=4.71-4.74 (m, 1H), 4.39-4.41 (m, 1H), 3.97-4.01 (m, 1H), 3.89-3.92 (m, 1H), 0.93 (s, 9H), 3.91 (s, 9H), 0.22 (s, 3H), 0.18 (s, 3H), 0.10 (s, 6H).

0.65 kg of 8 had been converted to compound 18 in two batches.(0.1 kg, 0.55 kg).

| | Conditions | | Product | | |
|---|---|---|---|---|---|
| Cpd. 7 | Temp. | Time | Weight | Purity | Yield |
| (kg) | (° C.) | (hrs) | (g) | (%) | (%) |
| 0.1 | 25 | 1 | 600 | 97.48 | 70 |
| 0.55 | 25 | 1 | | | |

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications are cited herein, the disclosures of which are incorporated by reference in their entireties.

We claim:

1. A method for making a compound of Formula IV: 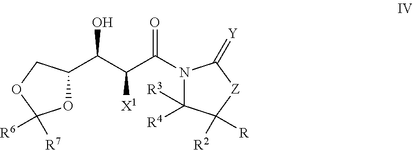
comprising reacting a 4-(S)-oxazolidinone of Formula II with an X¹-substituted acetyl group that includes a leaving group attached to the carbonyl moiety to form an imide of Formula III, 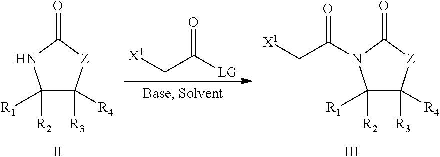
wherein:
X¹ is Cl, Br, or I,
Y is O or S,
Z is O, S, N-alkyl, N-aryl, N-alkylaryl, or N-arylalkyl;
R¹, R², R³ and R⁴ are, independently, selected from the group consisting of H, C₁₋₁₀ alkyl, C₁₋₁₀ branched alkyl, C₃₋₆ cycloalkyl, C₁₋₄(alkyl)aryl, benzyl, C₁₋₃(alkyl)C—O-aryl, CH₂OBz (where Bz=benzoyl), aryl, heteroaryl and C₃₋₆ heterocycloalkyl;
R¹ and R² or R³ and R⁴ can optionally be linked to form a C₃₋₈ cycloalkyl, alkyl substituted C₃₋₈ cycloalkyl or C₃₋₈ cycloalkyl fused to aryl;
LG is a leaving group, and
converting the compound of Formula III to a compound of Formula IV, by reacting the compound of Formula III, under basic conditions, with (R)-2,2-disubstituted-1,3-dioxolane-4-carbaldehyde: 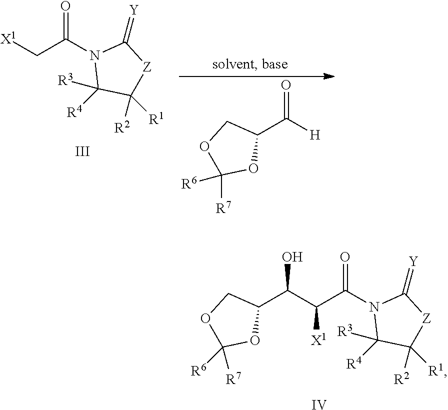
wherein X¹, R¹, R², R³, and R⁴ are as defined in claim 1, and R⁶ and R⁷ are, independently, selected from the group consisting of C₁₋₁₀ alkyl, aryl, C₁₋₄ (alkyl)aryl, such as benzyl, heteroaryl and C₁₋₄ (alkyl)heteroaryl; and
R⁶ and R⁷ optionally can be linked to form C₃₋₈ cycloalkyl, C₅₋₈ cycloheteroalkyl, alkyl substituted C₃₋₈ cycloalkyl and C₃₋₈ cycloalkyl fused to aryl.

2-6. (canceled)

7. The method of claim 1, further comprising the additional step of converting a compound of Formula IV: 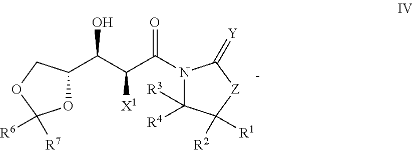
to a compound of Formula V: 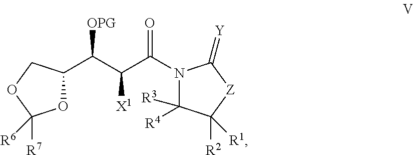
wherein R⁶ and R⁷ are, independently, selected from the group consisting of C₁₋₁₀ alkyl, aryl, C₁₋₄ (alkyl)aryl, such as benzyl, heteroaryl and —(C₁₋₄ alkyl)-heteroaryl; or R⁶ and R⁷ optionally can be linked to form C₃₋₈ cycloalkyl, C₅₋₈ cycloheteroalkyl, alkyl substitute C₃₋₈ cycloalkyl and C₃₋₈ cycloalkyl fused to aryl,
by protecting the OH group in Compound IV with a hydroxyl protecting group (PG) which is largely stable to both basic conditions and acidic conditions.

8. (canceled)

9. The method of claim 7, further comprising the additional step of converting a compound of Formula V to a compound of Formula VI, by removing the oxazolidinone chiral auxiliary on Formula V with an alkoxide of the formula R⁵OM, wherein M is a metal or a quaternary ammonium salt, and R⁵ is selected from the group consisting of C₁₋₁₀ alkyl, C₁₋₁₀ branched alkyl C₃₋₆ cycloalkyl and alkyl substituted C₃₋₈ cycloalkyl.

10-11. (canceled)

12. The method of claim 9, further comprising the additional step of converting a compound of Formula VI: 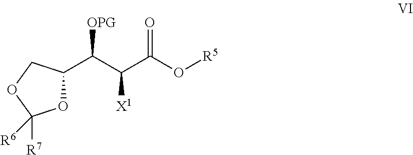
to a compound of Formula VII 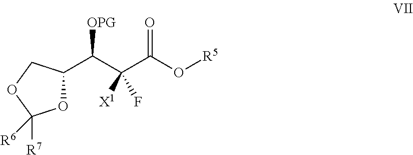
by reacting the compound of Formula VI with a base that is too hindered to displace X¹, and an electrophilic fluorine reagent.

13. The method of claim 12, wherein the base is n-butyl lithium, lithium bis(trimethylsilyl)amide, lithium diisopropylamide, lithium tetramethylpiperidide or sec-butyl lithium.

14. The method of claim 11, wherein the electrophilic fluorine reagent is selected from the group consisting of N-fluoro-o-benzenedisulfonimide (NFOBS), N-fluorobenzenesulfonimide (NFSI), 2-fluoro-3,3-dimethyl-2,3-dihydro-1,2-benzisothiazole 1,1-dioxide [124170-23-6], 1-fluoro-4-hydroxy-1,4-diazoniabicyclo[2,2,2]octane bis(tetrafluoroborate) on aluminum oxide (Accufluor), N-fluoropyridinium salts: [178439-26-4], [107264-00-96], [109705-14-8], [107264-09-5], [107263-95-6], [140623-89-8], [130433-68-0] and 1-chloromethyl-4-fluoro-1,4-diazoniabicyclo[2.2.2]octane bis(tetrafluoroborate) (Selectfluor).

15. The method of claim 12, further comprising the additional step of converting a compound of Formula VII: 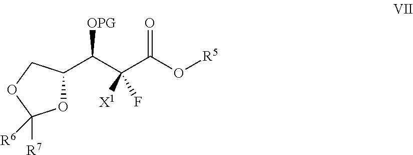
to a ribonolactone of Formula VIII 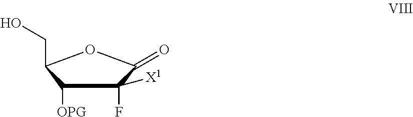
by contacting a compound of Formula VII with an acid catalyst in a polar aprotic solvent, which solvent can optionally comprise water.

16. (canceled)

17. The method of claim 15, further comprising the additional step of converting the ribonolactone of Formula VIII: 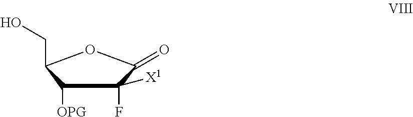
to the ribolactone of Formula I: 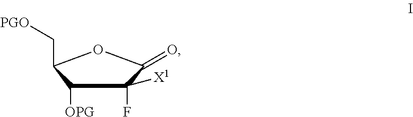
by protecting the hydroxyl group, wherein the base is a secondary or tertiary amine, and a protective group reagent is a reagent capable of forming PG, in the presence of a base.

18-19. (canceled)

20. A method for forming compounds of Formula XIII 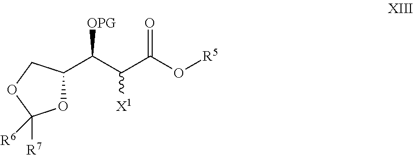
comprising reacting an alpha halo acetate ester of Formula X: 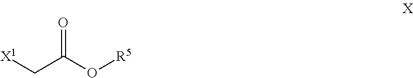
with (R)-2,2-disubstituted-1,3-dioxolane-4-carbaldehyde: 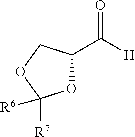
in the presence of a base, to provide compounds of Formula XI: 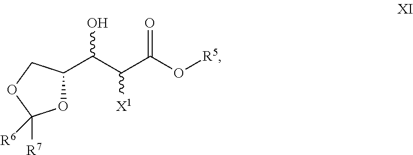
in which the 3-OH is predominately in the R stereochemical configuration, wherein X¹, R⁵, R⁶, and R⁷ are as defined above in claim 7,
wherein X¹ is Cl, Br, or I,
R⁵ is selected from the group consisting of C₁₋₁₀ alkyl, C₁₋₁₀ branched alkyl C₃₋₆ cycloalkyl and alkyl substituted C₃₋₈ cycloalkyl,
R⁶ and R⁷ are, independently, selected from the group consisting of C₁₋₁₀ alkyl, aryl, C₁₋₄ (alkyl)aryl, such as benzyl, heteroaryl and —(C₁₋₄ alkyl)-heteroaryl; or R⁶ and R⁷ optionally can be linked to form C₃₋₈ cycloalkyl, C₅₋₈ cycloheteroalkyl, alkyl substitute C₃₋₈ cycloalkyl and C₃₋₈ cycloalkyl fused to aryl,
base is a sterically hindered base, and the solvent is an aprotic solvent,
and protecting the free hydroxyl group on the compounds of Formula XI, to form a compound of Formula XIII: 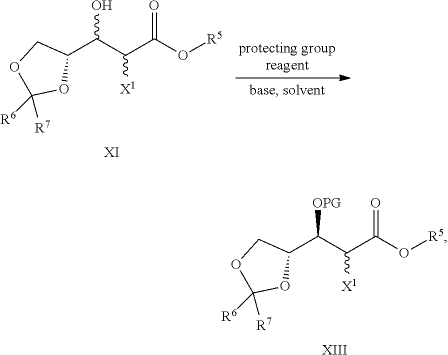
wherein PG is a hydroxyl protecting group, base is a sterically-hindered base, a protective group reagent is a reagent capable of forming PG, in the presence of a base, and the solvent is a polar, aprotic solvent.

21. A method for preparing compounds of Formula XIII 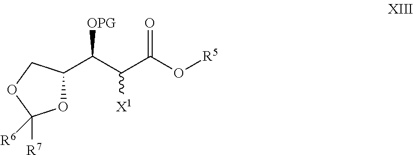
in which the 3-OH is predominately in the R stereochemical configuration, comprising:
reacting compounds of Formula XII: 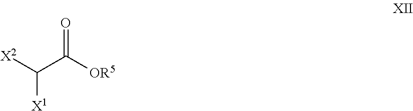
with a compound of formula: 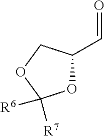
in the presence of a metal or a borane, in a polar aprotic solvent, to from a compound of Formula XI: 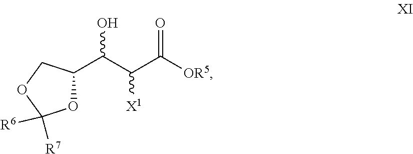
wherein X² is a halogen selected from the group consisting of Cl, Br, and I,
X¹ is Cl, Br, or I,
R⁵ is selected from the group consisting of C₁₋₁₀ alkyl, C₁₋₁₀ branched alkyl C₃₋₆ cycloalkyl and alkyl substituted C₃₋₈ cycloalkyl,
R⁶ and R⁷ are, independently, selected from the group consisting of C₁₋₁₀ alkyl, aryl, C₁₋₄ (alkyl)aryl, such as benzyl, heteroaryl and —(C₁₋₄ alkyl)-heteroaryl; or R⁶ and R⁷ optionally can be linked to form C₃₋₈ cycloalkyl, C₅₋₈ cycloheteroalkyl, alkyl substitute C₃₋₈ cycloalkyl and C₃₋₈ cycloalkyl fused to aryl, and
protecting the free hydroxyl group on the compounds of Formula XI, to form a compound of Formula XIII: 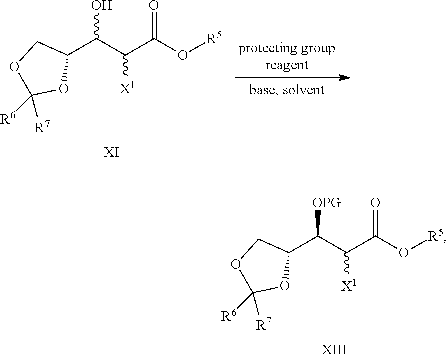
wherein PG is a hydroxyl protecting group, base is a sterically-hindered base, a protective group reagent is a reagent capable of forming PG, in the presence of a base, and the solvent is a polar, aprotic solvent.

22. (canceled)

23. The method of claim 20, further comprising separating the protected compounds of Formula XIII into individual diastereomers, and isolating a compound with the desired stereochemistry.

24. The method of claim 23, further comprising converting compounds of Formula XIII into dihalo intermediates of Formula XIV in the presence of an electrophilic fluorine reagent, and under the action of a base which is too hindered to displace X¹, in a polar, aprotic solvent: 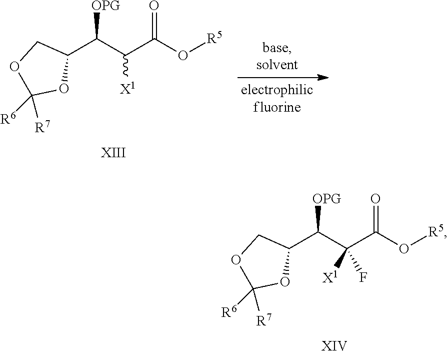
wherein:
X¹ is Cl, Br, or I,
R⁵ is selected from the group consisting of C₁₋₁₀ alkyl, C₁₋₁₀ branched alkyl C₃₋₆ cycloalkyl and alkyl substituted C₃₋₈ cycloalkyl,
R⁶ and R⁷ are, independently, selected from the group consisting of C₁₋₁₀ alkyl, aryl, C₁₋₄ (alkyl)aryl, such as benzyl, heteroaryl and —(C₁₋₄ alkyl)-heteroaryl; or R⁶ and R⁷ optionally can be linked to form C₃₋₈ cycloalkyl, C₅₋₈ cycloheteroalkyl, alkyl substitute C₃₋₈ cycloalkyl and C₃₋₈ cycloalkyl fused to aryl,
PG is a hydroxyl protecting group, and
base is a sterically-hindered base.

25. The method of claim 24, further comprising the additional step of converting a compound of Formula XIV to a ribonolactone by contacting a compound of Formula XIV with an acid catalyst in a polar aprotic solvent, which solvent can optionally comprise water.

26-28. (canceled)

29. A method for forming compounds of Formula XVIII: 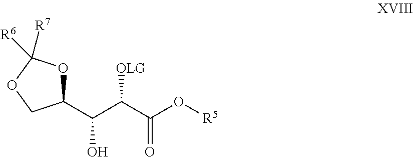
comprising oxidizing compounds XV with an oxidizing reagent, followed by condensation with an α,β-unsaturated ester formation reagent: 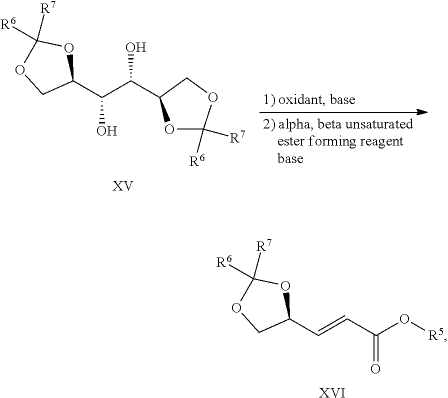
wherein:
R⁵ is selected from the group consisting of C₁₋₁₀ alkyl, C₁₋₁₀ branched alkyl C₃₋₆ cycloalkyl and alkyl substituted C₃₋₈ cycloalkyl,
R⁶ and R⁷ are, independently, selected from the group consisting of C₁₋₁₀ alkyl, aryl, C₁₋₄ (alkyl)aryl, such as benzyl, heteroaryl and —(C₁₋₄ alkyl)-heteroaryl; or R⁶ and R⁷ optionally can be linked to form C₃₋₈ cycloalkyl, C₅₋₈ cycloheteroalkyl, alkyl substitute C₃₋₈ cycloalkyl and C₃₋₈ cycloalkyl fused to aryl, and
the oxidizing agent is an inorganic or organic oxidizing reagent that includes, but is not limited to, NaIO₄, HIO₄, KIO₄, LiIO₄, Pb(OAc)₄, NaBiO₃, CrO₂Cl₂, PhI(OAc)₂,
and the α, β-unsaturated ester formation reagent is alkyl trialkylphosphonoacetate or (carbalkoxymethylene)triarylphosphorane,
converting the compound of Formula XVI to a compound of Formula XVII by converting the carbon-carbon double bond is converted to dihydroxyl groups by reaction with a dihydroxylation reagent: 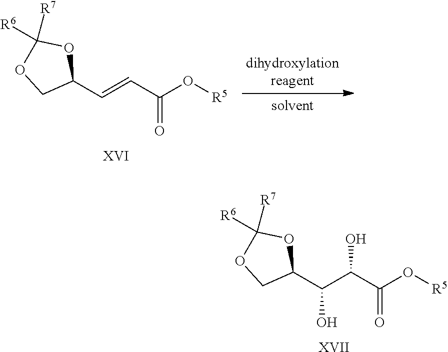
wherein the dihydroxylation agent is an inorganic or organic oxidizing reagent,
R⁵ is selected from the group consisting of C₁₋₁₀ alkyl, C₁₋₁₀ branched alkyl C₃₋₆ cycloalkyl and alkyl substituted C₃₋₈ cycloalkyl,
R⁶ and R⁷ are, independently, selected from the group consisting of C₁₋₁₀ alkyl, aryl, C₁₋₄ (alkyl)aryl, such as benzyl, heteroaryl and —(C₁₋₄ alkyl)-heteroaryl; or R⁶ and R⁷ optionally can be linked to form C₃₋₈ cycloalkyl, C₅₋₈ cycloheteroalkyl, alkyl substitute C₃₋₈ cycloalkyl and C₃₋₈ cycloalkyl fused to aryl, and
converting the compound of Formula XVII to a compound of Formula XVIII by converting the α-hydroxyl group (i.e., the hydroxyl group alpha to the —CO(OR⁵ moiety) into a leaving group (LG) by reaction with a leaving group reagent:

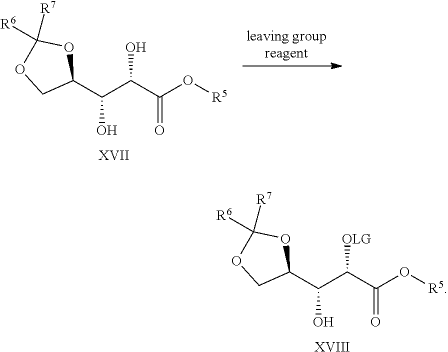

30-31. (canceled)

32. The method of claim 30, further comprising displacing the leaving group of compound XVIII 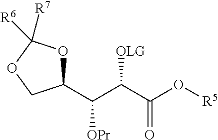
with a halogen reagent RX¹ to form a compound of Formula XIX: 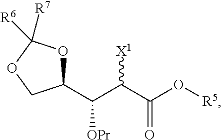
wherein RX¹ is an inorganic or organic salts of a halide, in which X¹ is Cl, Br or I,
Pr is a hydroxyl protecting group,
R is Li, Na, K, or NBu₄,
R⁵ is selected from the group consisting of C₁₋₁₀ alkyl, C₁₋₁₀ branched alkyl C₃₋₆ cycloalkyl and alkyl substituted C₃₋₈ cycloalkyl, and
R⁶ and R⁷ are, independently, selected from the group consisting of C₁₋₁₀ alkyl, aryl, C₁₋₄ (alkyl)aryl, such as benzyl, heteroaryl and —(C₁₋₄ alkyl)-heteroaryl; or R⁶ and R⁷ optionally can be linked to form C₃₋₈ cycloalkyl, C₅₋₈ cycloheteroalkyl, alkyl substitute C₃₋₈ cycloalkyl and C₃₋₈ cycloalkyl fused to aryl.

33. The method of claim 32, further comprising protecting the free hydroxyl group.

34. The method of claim 29, further comprising protecting the free hydroxyl group.

35. (canceled)

36. The method of claim 34, further comprising displacing the leaving group of compound XVIII with a halogen reagent RX¹ to form a compound of Formula XIX, wherein RX¹ is an inorganic or organic salts of a halide, in which X¹ is Cl, Br or I, and R is Li, Na, K, or NBu_{4:}

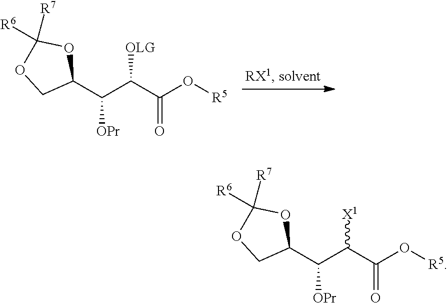

37. The method of claim 33, further comprising separating the protected compounds of Formula XIX into individual diastereomers, and isolating a compound with the desired stereochemistry.

38. The method of claim 34, further comprising reacting the protected compounds of Formula XIX with an electrophilic fluorine reagent, under the action of a base which is too hindered to displace X¹, in a polar, aprotic solvent, to form a compound with both an X¹ and F moiety on the same carbon.

39. The method of claim 38, further comprising the additional step of converting the product to a ribonolactone by contacting the product with an acid catalyst in a polar aprotic solvent, which solvent can optionally comprise water.

40. (canceled)

41. The method of claim 40, further comprising protecting the hydroxyl group on the ribonolactone.

42. (canceled)

43. A method for forming a compound of Formula XXIII: 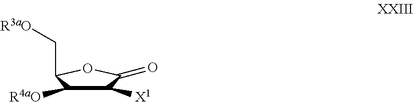
comprising:
a) reacting a compound of Formula XX: 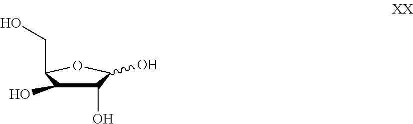
with a ketal or acetal-forming reagent to form a ketal which includes R⁶ and R⁷ groups to protect all four of the hydroxyl groups as ketals or acetals,
wherein R⁶ and R⁷ are, independently, selected from the group consisting of C₁₋₁₀ alkyl, aryl, C₁₋₄ (alkyl)aryl, such as benzyl, heteroaryl and —(C₁₋₄ alkyl)-heteroaryl; or R⁶ and R⁷ optionally can be linked to form C₃₋₈ cycloalkyl, C₅₋₈ cycloheteroalkyl, alkyl substitute C₃₋₈ cycloalkyl and C₃₋₈ cycloalkyl fused to aryl,
b) regioselectively deprotecting the ketal or acetal at the 3′- and 5′-position, and
c) benzylating the hydroxyl groups at the 3′- and 5′-positions, to form a compound of Formula XXI: 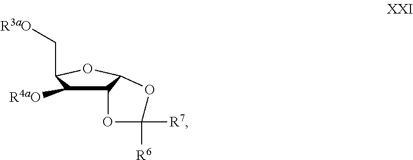
wherein R^{3a} and R^{4a} are benzyl, 4-halobenzyl, 4-methylbenzyl, 4-methoxybenzyl, 4-nitrobenzyl, or 2,4-dimethylbenzyl groups,
removing the ketal or acetal group on the compound of formula XXI by acid hydrolysis under conditions that do not cleave the protecting groups at the 3′- and 5′-positions, and regioselective oxidation of the 1-hydroxyl group to form a compound of formula XXII: 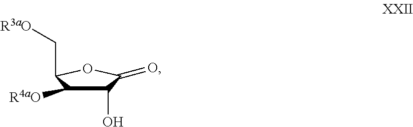
and
converting the 2-hydroxyl group of the 2-hydroxylactone of formula XXII to a halide, to form the corresponding 2-halolactone of formula XXIII: 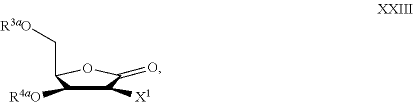
wherein X¹ is a halide.

44. The method of claim 43, wherein the compound XX is D-xylose, and all four hydroxyl groups are protected using 2,2′ -dimethoxypropane, or 2-methoxypropene in acetone in the presence of catalytic amount of H₂SO₄, HClO₄, or HCl, optionally along with CuSO₄, followed by selective deprotection of the 3′,5′-acetonide group with a catalytic amount of an acid strong enough to hydrolyze the 3′,5′-acetonide group.

45. The method of claim 44, wherein the acid used for the acid hydrolysis step is selected from the group consisting of acetic acid, formic acid, trifluoroacetic acid (TFA), trichloroacetic acid (TCA), TsOH, pyridinium p-toluenesufonate (PPTS), camphorsulfonic acid, and H₂SO₄.

46-49. (canceled)

50. The method of claim 43, further comprising converting the 2-halolactone of formula XXIII: 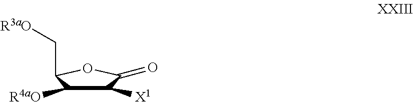
to a 2-halo, 2-fluorolactone of formula XXIV: 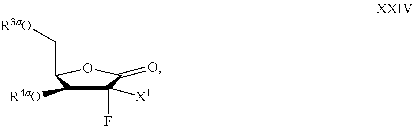
by reacting the halolactone with an electrophilic fluoride reagent in the presence of a strong, sterically-hindered base.

51-52. (canceled)

53. The method of claim 50, further comprising removing the 3- and 5-protecting groups from the 2-chloro-2-fluorolactone of formula XXIV: 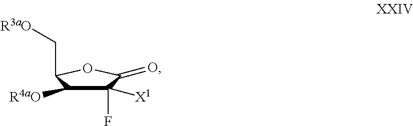
and regioselectively protecting two of the three resulting free hydroxyl groups as a ketal or acetal, to provide an acyclic ester of formula XXV: 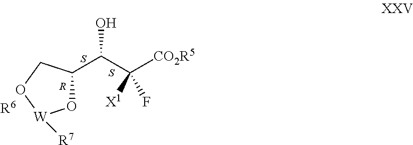
wherein W is carbon, boron, silicon, or phosphorus, and
X¹ is Cl, Br, or I,
R⁵ is selected from the group consisting of C₁₋₁₀ alkyl, C₁₋₁₀ branched alkyl C₃₋₆ cycloalkyl and alkyl substituted C₃₋₈ cycloalkyl,
R⁶ and R⁷ are, independently, selected from the group consisting of C₁₋₁₀ alkyl, aryl, C₁₋₄ (alkyl)aryl, such as benzyl, heteroaryl and —(C₁₄ alkyl)-heteroaryl; or R⁶ and R⁷ optionally can be linked to form C₃₋₈ cycloalkyl, C₅₋₈ cycloheteroalkyl, alkyl substitute C₃₋₈ cycloalkyl and C₃₋₈ cycloalkyl fused to aryl, and
R^{3a} and R^{4a} are benzyl, 4-halobenzyl, 4-methylbenzyl, 4-methoxybenzyl, 4-nitrobenzyl, or 2,4-dimethylbenzyl groups.

54. The method of claim 53, further comprising converting the hydroxyl group into a leaving group, and then performing an appropriate nucleophilic substitution reaction to replace the leaving group with a hydroxyl group, to provide a corresponding β(R)-hydroxyl ester of formula XXVI

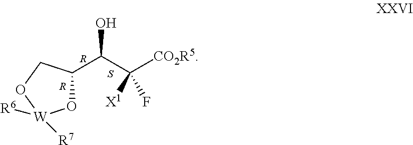

55-56. (canceled)

57. The method of claim 54, further comprising:
i) protecting the unprotected hydroxyl group of the β(R)-hydroxyl ester of formula XXVI: 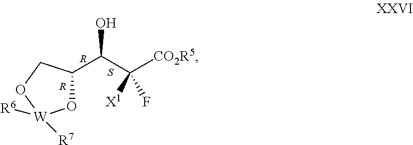
and
ii) deprotecting the —O—W(R⁶R⁷)O— protecting group using acid hydrolysis, to convert the ester into the corresponding lactone, and
iii) protecting the remaining unprotected primary hydroxyl group to form a cyclic diprotected lactone compound of Formula I:

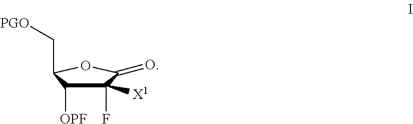

58-64. (canceled)
